SEQUENCE LISTING (52 sequences)

SEQ ID NO: 1 - DNA, 2800 bp, Homo Sapiens
  CDS (338)...(2395)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 2 - protein, 685 aa, Homo Sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAMCSPRDSMYQSVCLISEERNECVIATEV

SEQ ID NO: 3 - DNA, 2055 bp, Homo Sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggta

SEQ ID NO: 4 - protein, 720 aa, Mus Musculus
MGRRSALALAVVSALLCQVWSSGVFELKLQEFVNKKGLLGNRNCCRGGSGPPCACRTFFRVCLKHQASVSPEPPCTYGSAVTPVLGVDSFSLPDGAGIDPAFSNPIRFPFGFTWPGTFSLIIEALHTDSPDDLATENPERLISRLTTQRHTVGEEWSQDLHSSGRTDLRYSYRFVCDEHYYGEGCSVFCRPRDDAFGHFTCGDRGEKMCDPGWKGQYCTDPICLPGCDDQHGYCDKPGECKCRVGWQGRYCDECIRYPGCLHGTCQQPWQCNCQEGWGGLFCNQDLNYCTHHKPCRNGATCTNTGQGSYTCSCRPGYTGANCELEVDECAPSPCKNGASCTDLEDSFSCTCPPGFYGKVCELSAMTCADGPCFNGGRCSDNPDGGYTCHCPLGFSGFNCEKKMDLCGSSPCSNGAKCVDLGNSYLCRCQAGFSGRYCEDNVDDCASSPCANGGTCRDSVNDFSCTCPPGYTGKNCSAPVSRCEHAPCHNGATCHQRGQRYMCECAQGYGGPNCQFLLPEPPPGPMVVDLSERHMESQGGPFPWVAVCAGVVLVLLLLLGCAAVVVCVRLKLQKHQPPPEPCGGETETMNNLANCQREKDVSVSIIGATQIKNTNKKADFHGDHGAKKSSFKVRYPTVDYNLVRDLKGDEATVRDTHSKRDTKCQSQSSAGEEKIAPTLRGGEIPDRKRPESVYSTSKDTKYQSVYVLSAEKDECVIATEV

SEQ ID NO: 5 - protein, 713 aa, Rattus Norvegicus
MGRRSALALAVVSALLCQVWSSGVFELKLQEFVNKKGLLGNRNCCRGGSGPPCACRTFFRVCLKHYQASVSPEPPCTYGSAVTAVLGVDSFSLPDGAGIDPAFSNPIRFPFGFTWPGTFSLIIEALHTDSPDDLATENPERLISRLTTQRHTVGEEWSQDLHSSGRTDLRYSYRFVCDEHYYGEGCSVFCRPRDDAFGHFTCGERGEKMCDPGWKGQYCTDPICLPGCDDQHGYCDKPGECKCRVGWQGRYCDECIRYPGCLHGTCQQPWQCNCQEGWGGLFCNQDLNYCTHHKPCRNGATCTNTGQGSYTCSCRPGYTGANCELEVDECAPSPCKNGGSCTDLEDSYSCTCPPGFYGKVCELSAMTCADGPCFNGGRCSDNPDGGYTCHCPAGFSGFNCEKKIDLCSSSPCSNGAKCVDLGNSYLCRCQTGFSGRYCEDNVDDCASSPCANGGTCRDSVNDFSCTCPPGYTGKNCSAPVSRCEHAPCHNGATCHQRGQRYMCECAQGYGGANCQFLLPEPPPDLIVAAQGGSFPWVAVCAGVVLVLLLLLGCAAVVVCVRLKLQKHQPPPDPCGGETETMNNLANCQREKDVSVSIIGATQIKNTNKKADFHGDHGADKSSFKARYPTVDYNLIRDLKGDEATVRDAHSKRDTKCQSQGSAGEEKSTSTLRGGEVPDRKRPESVYSTSKDTKYQSVYVLSAEKDECVIATEV

SEQ ID NO: 6 - protein, 157 aa, Homo Sapiens
ENSYSCTCPPGFYGKICELSAMTCADGPCFNGGRCSDSPDGGYSCRCPVCYSGFNCEKKIDYCSSSPCSNGAKCVDLGDAYLCRCQAGFSCRHCDDNVDDCASSPCANGGTCRDGVNDFSCTCPPGYTGRNCSAPASRCEHAPCHNGATCHERGHRY

SEQ ID NO: 7 - protein, 721 aa, Xenopus Laevis
MGQQRMLTLLVLSAVLCQISCSGLFELRLQEFVNKKGLLGNMNCCRPGSLASLQRCECKTFFRICLKHYQSNVSPEPPCTYGGAVTPVLGTNSFVVPESSNADPTFSNPIRFPFGFTWPGTFSLIIEAIHADSADDLNTENPERLISRLATQRHLTVGEQWSQDLHSSDRTELKYSYRFVCDEYYYGEGCSDYCRPRDDAFGHFSCGEKGENLCNPGWKGLYCTEPICLPGCDEHHGYCDKPGECKCRVGWQGRYCDECIRYPGCLHGTCQQPWQCNCQEGWGGLFCNQDLNYCTHHKPCENGATCTNTGQGSYTCSCRPGYTGSNCEIEVNECDANPCKNGGSCSDLENSYTCSCPPGFYGKNCELSAMTCADGPCFNGGRCADNPDGGYICPCPVGYSGFNCEKKIDYCSSNPCANGARCEDLGNSYICQCQEGFSGRNCDDNLDDCTSFPCQNGGTCQDGINDYSCTCPPGYIGKNCSMPITKCEHNPCHNGATCHERNNRYVCQCARGYGGNNCQFLLPEEKPVVVDLTEKYTEGQSGQFPWIAVCAGIVLVLMLLLGCAAVVVCVRVRVQKRRHQPEACRGESKTMNNLANCQREKDISVSFIGTTQIKNTNKKIDFLSESNNEKNGYKPRYPSVDYNLVHELKNEDSPKEERSKCEAKCSSNDSDSEDVNSVHSKRDSSERRRPDSAYSTSKDTKYQSVYVISDEKDECIIATEV

SEQ ID NO: 8 - protein, 729 aa, Gallus Gallus
MGGRFLLTLALLSALLCRCQVDGSGVFELKLQEFVNKKGLLSNRNCCRGGGPGGAGQQQCDCKTFFRVCLKHYQASVSPEPPCTYGSAITPVLGANSFSVPDGAGGADPAFSNPIRFPFGFTWPGTFSLIIEALHTDSPDDLTTENPERLISRLATQRHLAVGEEWSQDLHSSGRTDLKYSYRFVCDEHYYGEGCSVFCRPRDDRFGHFTCGERGEKVCNPGWKGQYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGSSCEIEINECDANPCKNGGSCTDLENSYSCTCPPGFYGKNCELSAMTCADGPCFNGGRCTDNPDGGYSCRCPLGYSGFNCEKKIDYCSSSPCANGAQACVDLGNSYICQCQAGFTGRHCDDNVDDCASFPCVNGGTCQDGVNDYSCTCPPGYNGKNCSTPVSRCEHNPCHNGATCHERSNRYVCECARGYGGLNCQFLLPEPPQGPVIVDFTEKYTEGQNSQFPWIAVCAGIILVLMLLLGCAAIVVCVRLKVQKRHHQPEACRSETETMNNLANCQREKDISISVIGATQIKNTNKKVDFHSDNSDKNGYKVRYPSVDYNLVHELKNEDSVKEEHGKCEAKCETYDSEAEEKSAVQLKSSDTSERKRPDSVYSTSKDTKYQSVYVISEEKDECIIATEV

SEQ ID NO: 9 - protein, 717 aa, Danio Rerio
MGRLMIAVLLCVMISQGFCSGVFELKLQEFLNKKGVTGNANCCKGSAAEGHQCECKTFFRICLKHYQANVSPDPPCTYGGAVTPVLGSNSFQVPESFPDSSFTNPIPFAFGFTWPGTFSLIIEALHTDSTDDLSTENPDRLISRMTTQRHLTVGEEWSQDLQVGGRTELKYSYRFVCDEHYYGEGCSVFCRPRDDTFGHFTCGERGEIICNSGWKGQYCTEPICLPGCDEDHGFCDKPGECKCRVGFSGKYCDDCIRYPGCLHGTCQQPWQCNCQEGWGGLFCNQDLNYCTHHKPCQNGATCTNTGQGSYTCSCRPGFTGDSCEIEVNECSGSPCRNGGSCTDLENTYSCTCPPGFYGRNCELSAMTCADGPCFNGGHCADNPEGGYFCQCPMGYAGFNCEKKIDHCSSNPCSNDAQCLDLVDSYLCQCPEGFTGTHCEDNIDECATYPCQNGGTCQDGLSDYTCTCPPGYTGKNCTSAVNKCLHNPCHNGATCHEMDNRYVCACIPGYGGRNCQFLLPENPQGQAIVEGADKRYSYEEDDGGFPWTAVCAGIILVLLVLIGGSVFVIYIRLKLQQRSQQIDSHSEIETMNNLTNNRSREKDLSVSIIGATQVKNINKKVDFQSDGDKNGFKSRYSLVDYNLVHELKQEDLGKEDSERSEATKCEPLDSDSEEKHRNHLKSDSSERKRTESLCKDTKYQSVFVLSEEKDECIIATEV

SEQ ID NO: 10 - protein, 642 aa, Xenopus Laevis
MASPLLLVYVAATLCLPLVYPAGVFELKIHSFSTPRPACAAGKSCNIFFRVCLKHAQPVVSPDPPCTFGSAVSDILPSDSKAITDSSPIRVPFHFKWPGIFSLIIESWTTNSAEQSTENPDNLLSRLATRRRLSIGEDWSQDIHLGQQSELRYSYHVSCDEHYYGDSCSDYCRPRDDNFGHYTCDEQGNRLCMSGWKGEYCAEPICLPGCSESHGFCELPGECKCRMGWQGELCDECLRYPGCQHGSCSQPWECICQEGWGGLFCNQDLNYCTNHQPCRNGASCINIGQGSYSCSCRAGFTGTNCEIDINECASNPCKNGGSCNDLENDYECVCPRGFYGKNCDISAMTCEDGPCFNGGTCIEKSSGVGYVCRCPPNYHGSNCEKKIDRCTNSPCLNGGQCLDMGRNVLCKCRPGPSGPRCELNIDDCASSPCANGGTCVDAVNSYTCSCTLGYGGKDCTLRVDACSSKPCKNGGTCYTKFTGNVCQCPTGFMGTSCEFRVHDPTPASHRADSSNTLTMVVCLGLLTFFLLGCGVFMVMRGMRRGHFNEKGRVNNDLEPKNNLIEKEPHFKMPNPDYLREKSSSKQKLLQGSESEEERSGRRTDRKPDTKQCNPTSRYPEDGAYHPIYILPEPEQCIFATEV

SEQ ID NO: 11 - protein, 830 aa, Drosophila Melanogaster
MHWIKCLLTAFICFTVIVQVHSSGSFELRLKYFSNDHGRDNEGRCCSGESDGATGKCLGSCKTRFRVCLKHYQATIDTTSQCTYGDVITPILGENSVNLTDAQRFQNKGFTNPIQFPFSFSWPGTFSLIVEAWHDTNNSGNARTNKLLIQRLLVQQVLEVSSEWKTNKSESQYTSLEYDFRVTCDLNYYGSGCAKFCRPRDDSFGHSTCSETGEIICLTGWQGDYCHIPKCAKGCEHGHCDKPNQCVCQLGWKGALCNECVLEPNCIHGTCNKPWTCICNEGWGGLYCNQDLNYCTNHRPCKNGGTCFNTGEGLYTCKCAPGYSGDDENEIYSCDADVNPCQNGGTCIDEPHTKTGKCHCANGWSGKMCEEKVLTCSDKPCHQGICRNVRPGLGSKGQGYQCECPIGYSGPNCDLQLDNCSPNPCINGGSCQPSGKCICPAGFSGTRCETNIDDCLGHQCENGGTCIDMVNQYRCQCVPGFHGTHCSSKVDLCLIRPCANGGTCLNLNNDYQCTCRAGFTGKDCSVDIDECSSGPCHNGGTCMNRVNSFECVCANGFRGKQCDEESYDSVTFDAHQYGATTQARADGLTNAQVVLIAVFSVAMPLVAVIAACVVFCMKRKRKRAQEKDDAEARKQNEQNAVATMHHNGSGVGVALASASLGGKTGSNSGLTFDGGNPNIIKNTWDKSVNNICASAAAAAAAAAAADECLMYGGYVASVADNNNANSDFCVAPLQRAKSQKQLNTDPTLMHRGSPAGSSAKGASGGGPGAAEGKRISVLGEGSYCSQRWPSLAAAGVAGACSSQLMAAASAAGTDGTAQQQRSVVCGTPHM

SEQ ID NO: 12 - DNA, 20 bp, Homo Sapiens
agcgcctctggctgggcgct

SEQ ID NO: 13 - DNA, 20 bp, Homo Sapiens
cggccagaggccttgccacc

SEQ ID NO: 14 - DNA, 20 bp, Homo Sapiens
ttgcgctcccggctggagcc

SEQ ID NO: 15 - DNA, 20 bp, Homo Sapiens
atgcggcttggacctcggtt

SEQ ID NO: 16 - DNA, 21 bp, Homo Sapiens
tgccgccatccctcggggcgt

SEQ ID NO: 17 - DNA, 20 bp, Homo Sapiens
ggacgctgccgccatcccct

SEQ ID NO: 18 - DNA, 28 bp, Homo Sapiens
ggacgctgccgccatcccctcggggcgt

SEQ ID NO: 19 - DNA, 20 bp, Homo Sapiens
tcaatctggctctgttcgcg

SEQ ID NO: 20 - DNA, 26 bp, Homo Sapiens
cgctctctccacccgcgggccctcaa

SEQ ID NO: 21 - DNA, 171 bp, Homo Sapiens
  misc_feature (1)...(171): n = A,T,C or G
gcccaggcngaccctggtgtggactgtgagctggagctcagcgagtgtgacagcaacccctgtcgcantggaggcagctgtaaggaccangaggatggctaccactgcctgtgtcctccgggctactacggcntgcatcgtgaacacngcacctcttagctgngccgactc

SEQ ID NO: 22 - DNA, 20 bp, Homo Sapiens
ctcttctgttcctctggttg

SEQ ID NO: 23 - protein, 45 aa, Artificial Sequence
  DSL domain consensus sequence
XXCXXXYFGXXCXXXCHXRXDXFGRXXCXXXGXXXCXXGWXGXYC

SEQ ID NO: 24 - DNA, 3133 bp, Mus Musculus
  CDS (39)...(2099)
gtcgacccacgcgtccggtggagaggacaccccaagggatgacgcctgcgtcccggagcgcctgtcgctgggcgctactgctgctggcggtactgtggccgcagcagcgcgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggaggaaatggcatgaactgaacagactgtgaacccgccaagagtcgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagtaggtggagtgccacctgtagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttctgggcccttgagcctgggctccaccagtgcccactgctgccccgagaccaaccttgaagccgatcttcaaaaatcaataatatgaggttttgttttgtagtttattttggaatctagtattttgataatttaagaatcagaagcactggcctttctacattttataacattattttgtatataatgtgtatttataatatgaaaaaaaaaaaaaaaagggcggccgc

SEQ ID NO: 25 - protein, 686 aa, Mus Musculus
MTPASRSACRWALLLLAVLWPQQRAAGSGIFQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCQNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGGMPGKYPHSDKSLGEKVPLRLHSEKPECRISAICSPRDSMYQSVCLISEERNECVIATEV

SEQ ID NO: 26 - DNA, 2058 bp, Mus Musculus
atgacgcctgcgtcccggagcgcctgtcgctgggcgctactgctgctggcggtactgtggccgcagcagcgcgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggta

SEQ ID NO: 27 - DNA, 2800 bp, Homo Sapiens
  CDS (338)...(2395)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaaccagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 28 - protein, 685 aa, Homo Sapiens
MAAASRSASGWALLLLVALWQQRAAGSGVFQLQLQEFINQRGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAMCSPRDSMYQSVCLISEERNECVIATEV

SEQ ID NO: 29 - DNA, 2800 bp, Homo Sapiens
  CDS (338)...(457)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaactagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 30 - protein, 39 aa, Homo Sapiens
MAAASRSASGWALLLLVALWQQRAAGSGVFQLQLQEFIN

SEQ ID NO: 31 - DNA, 2800 bp, Homo Sapiens
  CDS (338)...(2395)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacaagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 32 - protein, 685 aa, Homo Sapiens
MAAASRSASGWALLLLVALWQQRAAGSGVFQLQLQEFINKRGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAMCSPRDSMYQSVCLISEERNECVIATEV

SEQ ID NO: 33 - DNA, 2800 bp, Homo Sapiens
  CDS (338)...(2395)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagttgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 34 - protein, 685 aa, Homo Sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLVVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAMCSPRDSMYQSVCLISEERNECVIATEV

SEQ ID NO: 35 - DNA, 2800 bp, Homo Sapiens
  CDS (338)...(2395)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcagcaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 36 - protein, 685 aa, Homo Sapiens
MAAASRSASGWALLLLVALWQQRAAGSGVFQLQLQEFINERGVLASGRPCEPGCRTFFRVCLKHFQAVVSPGPCTFGTVSTPVLGSNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAMCSPRDSMYQSVCLISEERNECVIATEV

SEQ ID NO: 37 - DNA, 2800 bp, Homo Sapiens
  CDS (338)...(2395)
gtcgacccacgcgtccggctgcgcgcaggccgggaacacgaggccaagagccgcagccccagccgccttggtgcagcgtacaccggcactagcccgcttgcagccccaggattagacagaagacgcgtcctcggcgcggtcgccgcccagccgtagtcacctggattacctacagcggcagctgcagcggagccagcgagaaggccaaaggggagcagcgtcccgagaggagcgcctcttttcagggaccccgccggctggcggacgcgcgggaaagcggcgtcgcgaacagagccagattgagggcccgcgggtggagagagcgacgcccgaggggatggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagaaaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatgtgctcccccagggactccatgtaccagtctgtgtgtttgatatcagaggagaggaatgaatgtgtcattgccacggaggtataaggcaggagcctacctggacatccctgctcagccccgcggctggaccttccttctgcattgtttacattgcatcctggatgggacgtttttcatatgcaacgtgctgctctcaggaggaggagggaatggcaggaaccggacagactgtgaacttgccaagagatgcaatacccttccacacctttgggtgtctgtctggcatcagattggcagctgcaccaaccagaggaacagaagagaagagagtggcagtagccccatggggcccggagctgctgtggcctccactggcatccgtgtttccaaaagtgcctttggcccagccaagggtgccaggcctaactggggcactcagggcagtgtgttggaaattccactgagggggaaatcaggtgctgcggccgc

SEQ ID NO: 38 - protein, 685 aa, Homo Sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGENCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAMCSPRDSMYQSVCLISEERNECVIATEV

SEQ ID NO: 39 - DNA, 3133 bp, Mus Musculus
  CDS (39)...(2099)
gtcgacccacgcgtccggtggagaggacaccccaagggatgacgcctgtgtcccggagcgcctgtcgctgggcgctactgctgctggcggtactgtggccgcagcagcgcgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggaggaaatggcatgaactgaacagactgtgaacccgccaagagtcgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagtaggtggagtgccacctgtagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttctgggcccttgagcctgggctccaccagtgcccactgctgccccgagaccaaccttgaagccgatcttcaaaaatcaataatatgaggttttgttttgtagtttattttggaatctagtattttgataatttaagaatcagaagcactggcctttctacattttataacattattttgtatataatgtgtatttataatatgaaaaaaaaaaaaaaaagggcggccgc

SEQ ID NO: 40 - protein, 686 aa, Mus Musculus
MTPVSRSACRWALLLLAVLWPQQRAAGSGIFQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCQNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGGMPGKYPHSDKSLGEKVPLRLHSEKPECRISAICSPRDSMYQSVCLISEERNECVIATEV

SEQ ID NO: 41 - DNA, 3133 bp, Mus Musculus
  CDS (39)...(2099)
gtcgacccacgcgtccggtggagaggacaccccaagggatgacgcctgcgacccggagcgcctgtcgctgggcgctactgctgctggcggtactgtggccgcagcagcgcgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggaggaaatggcatgaactgaacagactgtgaacccgccaagagtcgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagtaggtggagtgccacctgtagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttctgggcccttgagcctgggctccaccagtgcccactgctgccccgagaccaaccttgaagccgatcttcaaaaatcaataatatgaggttttgttttgtagtttattttggaatctagtattttgataatttaagaatcagaagcactggcctttctacattttataacattattttgtatataatgtgtatttataatatgaaaaaaaaaaaaaaaagggcggccgc

SEQ ID NO: 42 - protein, 686 aa, Mus Musculus
MTPATRSACRWALLLLAVLWPQQRAAGSGIFQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCQNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGGMPGKYPHSDKSLGEKVPLRLHSEKPECRISAICSPRDSMYQSVCLISEERNECVIATEV

SEQ ID NO: 43 - DNA, 3133 bp, Mus Musculus
  CDS (39)...(2099)
gtcgacccacgcgtccggtggagaggacaccccaagggatgacgcctgcgtcccggagcgcctgtcgctgggcgctactgctgctggcggtactgtggccgcagcagcacgctgcgggctccggcatcttccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggaggaaatggcatgaactgaacagactgtgaacccgccaagagtcgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagtaggtggagtgccacctgtagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttctgggcccttgagcctgggctccaccagtgcccactgctgccccgagaccaaccttgaagccgatcttcaaaaatcaataatatgaggttttgttttgtagtttattttggaatctagtattttgataatttaagaatcagaagcactggcctttctacattttataacattattttgtatataatgtgtatttataatatgaaaaaaaaaaaaaaaagggcggccgc

SEQ ID NO: 44 - protein, 686 aa, Mus Musculus
MTPASRSACRWALLLLAVLWPQQHAAGSGIFQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCQNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGGMPGKYPHSDKSLGEKVPLRLHSEKPECRISAICSPRDSMYQSVCLISEERNECVIATEV

SEQ ID NO: 45 - DNA, 3133 bp, Mus Musculus
  CDS (39)...(2099)
gtcgacccacgcgtccggtggagaggacaccccaagggatgacgcctgcgtcccggagcgcctgtcgctgggcgctactgctgctggcggtactgtggccgcagcagcgcgctgcgggctccggcatctaccagctgcggctgcaggagttcgtcaaccagcgcggtatgctggccaatgggcagtcctgcgaaccgggctgccggactttcttccgcatctgccttaagcacttccaggcaaccttctccgagggaccctgcacctttggcaatgtctccacgccggtattgggcaccaactccttcgtcgtcagggacaagaatagcggcagtggtcgcaaccctctgcagttgcccttcaatttcacctggccgggaaccttctcactcaacatccaagcttggcacacaccgggagacgacctgcggccagagacttcgccaggaaactctctcatcagccaaatcatcatccaaggctctcttgctgtgggtaagatttggcgaacagacgagcaaaatgacaccctcaccagactgagctactcttaccgggtcatctgcagtgacaactactatggagagagctgttctcgcctatgcaagaagcgcgatgaccacttcggacattatgagtgccagccagatggcagcctgtcctgcctgccgggctggactgggaagtactgtgaccagcctatatgtctttctggctgtcatgagcagaatggttactgcagcaagccagatgagtgcatctgccgtccaggttggcagggtcgcctgtgcaatgaatgtatcccccacaatggctgtcgtcatggcacctgcagcatcccctggcagtgtgcctgcgatgagggatggggaggtctgttttgtgaccaagatctcaactactgtactcaccactctccgtgcaagaatggatcaacgtgttccaacagtgggccaaagggttatacctgcacctgtctcccaggctacactggtgagcactgtgagctgggactcagcaagtgtgccagcaacccctgtcgaaatggtggcagctgtaaggaccaggagaatagctaccactgcctgtgtcccccaggctactatggccagcactgtgagcatagtaccttgacctgcgcggactcaccctgcttcaatgggggctcttgccgggagcgcaaccaggggtccagttatgcctgcgaatgcccccccaactttaccggctctaactgtgagaagaaagtagacaggtgtaccagcaacccgtgtgccaatggaggccagtgccagaacagaggtccaagccgaacctgccgctgccggcctggattcacaggcacccactgtgaactgcacatcagcgattgtgcccgaagtccctgtgcccacgggggcacttgccacgatctggagaatgggcctgtgtgcacctgccccgctggcttctctggaaggcgctgcgaggtgcggataacccacgatgcctgtgcctccggaccctgcttcaatggggccacctgctacactggcctctccccaaacaacttcgtctgcaactgtccttatggctttgtgggcagccgctgcgagtttcccgtgggcttgccacccagcttcccctgggtagctgtctcgctgggcgtggggctagtggtactgctggtgctcctggtcatggtggtagtggctgtgcggcagctgcggcttcggaggcccgatgacgagagcagggaagccatgaacaatctgtcagacttccagaaggacaacctaatccctgccgcccagctcaaaaacacaaaccagaagaaggagctggaagtggactgtggtctggacaagtccaattgtggcaaactgcagaaccacacattggactacaatctagccccgggactcctaggacggggcggcatgcctgggaagtatcctcacagtgacaagagcttaggagagaaggtgccacttcggttacacagtgagaagccagagtgtcgaatatcagccatttgctctcccagggactctatgtaccaatcagtgtgtttgatatcagaagagaggaacgagtgtgtgattgccacagaggtataaggcaggagcctactcagacacccagctccggcccagcagctgggccttccttctgcattgtttacattgcatcctgtatgggacatctttagtatgcacagtgctgctctgcggaggaggaggaaatggcatgaactgaacagactgtgaacccgccaagagtcgcaccggctctgcacacctccaggagtctgcctggcttcagatgggcagccccgccaagggaacagagttgaggagttagaggagcatcagttgagctgatatctaaggtgcctctcgaacttggacttgctctgccaacagtggtcatcatggagctcttgactgttctccagagagtggcagtggccctagtgggtcttggcgctgctgtagctcctgtgggcatctgtatttccaaagtgcctttgcccagactccatcctcacagctgggcccaaatgagaaagcagagaggaggcttgcaaaggataggcctcccgcaggcagaacagccttggagtttggcattaagcaggagctactctgcaggtgaggaaagcccgaggaggggacacgtgtgactcctgcctccaaccccagtaggtggagtgccacctgtagcctctaggcaagagttggtccttcccctggtcctggtgcctctgggctcatgtgaacagatgggcttagggcacgccccttttgccagccaggggtacaggcctcactggggagctcagggccttcatgctaaactcccaataagggagatggggggaagggggctgtggcctaggcccttccctccctcacacccatttctgggcccttgagcctgggctccaccagtgcccactgctgccccgagaccaaccttgaagccgatcttcaaaaatcaataatatgaggttttgttttgtagtttattttggaatctagtattttgataatttaagaatcagaagcactggcctttctacattttataacattattttgtatataatgtgtatttataatatgaaaaaaaaaaaaaaaagggcggccgc

SEQ ID NO: 46 - protein, 686 aa, Mus Musculus
MTPASRSACRWALLLLAVLWPQQRAAGSGIYQLRLQEFVNQRGMLANGQSCEPGCRTFFRICLKHFQATFSEGPCTFGNVSTPVLGTNSFVVRDKNSGSGRNPLQLPFNFTWPGTFSLNIQAWHTPGDDLRPETSPGNSLISQIIIQGSLAVGKIWRTDEQNDTLTRLSYSYRVICSDNYYGESCSRLCKKRDDHFGHYECQPDGSLSCLPGWTGKYCDQPICLSGCHEQNGYCSKPDECICRPGWQGRLCNECIPHNGCRHGTCSIPWQCACDEGWGGLFCDQDLNYCTHHSPCKNGSTCSNSGPKGYTCTCLPGYTGEHCELGLSKCASNPCRNGGSCKDQENSYHCLCPPGYYGQHCEHSTLTCADSPCFNGGSCRERNQGSSYACECPPNFTGSNCEKKVDRCTSNPCANGGQCQNRGPSRTCRCRPGFTGTHCELHISDCARSPCAHGGTCHDLENGPVCTCPAGFSGRRCEVRITHDACASGPCFNGATCYTGLSPNNFVCNCPYGFVGSRCEFPVGLPPSFPWVAVSLGVGLVVLLVLLVMVVVAVRQLRLRRPDDESREAMNNLSDFQKDNLIPAAQLKNTNQKKELEVDCGLDKSNCGKLQNHTLDYNLAPGLLGRGGMPGKYPHSDKSLGEKVPLRLHSEKPECRISAICSPRDSMYQSVCLISEERNECVIATEV

SEQ ID NO: 47 - DNA, 24 bp, Mus Musculus
ctccgggacgcaggcgtcatccct

SEQ ID NO: 48 - DNA, 29 bp, Mus Musculus
acaggcgctccgggacgcaggcgtcatcc

SEQ ID NO: 49 - DNA, 26 bp, Mus Musculus
ggtgtcctctccaccggacgcgtggg

SEQ ID NO: 50 - DNA, 22 bp, Mus Musculus
gtcctctccaccggacgcgtgg

SEQ ID NO: 51 - DNA, 20 bp, Homo Sapiens
gtttacattgcatcctggat

SEQ ID NO: 52 - protein, 20 aa, Artificial Sequence
  EGF-like domain consensus sequence
CXCXGXCXWXCXCXGWGXFC

(19) United States
(12) Patent Application Publication
(10) Pub. No.: US 2003/0180784 A1
(43) Pub. Date: Sep. 25, 2003

(54) Novel human Delta3 compositions and therapeutic and diagnostic uses therefor

(75) Inventors: Sean A. McCarthy, San Diego, CA (US); David P. Gearing, East Doncaster (AU)

Correspondence Address: MILLENNIUM PHARMACEUTICALS, INC., 75 Sidney Street, Cambridge, MA, 02139, US

(73) Assignee: Millennium Pharmaceuticals, Inc.

(21) Appl. No.: 10/417,719

(22) Filed: Apr. 17, 2003

Related U.S. Application Data
(63) Continuation of application No. 09/568,218, filed on May 9, 2000.
Application No. 09/568,218 is a continuation-in-part of application No. 08/872,855, filed on Jun. 11, 1997, now Pat. No. 6,121,045.
Application No. 08/872,855 is a continuation-in-part of application No. 08/832,633, filed on Apr. 4, 1997, now abandoned.

(51) Int. Cl. C12Q 1/68; G01N 33/53; C07H 21/04; C12P 21/02; C12N 5/06; C07K 14/705

(52) U.S. Cl. 435/6; 435/7.1; 435/69.1; 435/320.1; 435/325; 530/350; 536/23.5

(57) ABSTRACT

The invention provides nucleic acids encoding Delta3 proteins. Also provided are derivatives of Delta3 nucleic acids, polypeptides encoded thereby, and antibodies. Delta3 therapeutics, which are either antagonists or agonists of a Delta3 activity and which are capable of modulating the growth and/or differentiation of a cell, e.g., endothelial cell, are also provided herein. Furthermore, methods for treating or preventing diseases associated with an aberrant Delta3 activity and/or associated with abnormal cellular growth and/or differentiation, e.g., neurological disease or vascular disease, such as Agenesis of the Corpus Callosum with Peripheral Neuropathy (ACCPN), as well as diagnostic methods for detecting these diseases are disclosed.

### RELATED APPLICATIONS

This application is a continuation of U.S. Ser. No. 09/568,218 filed May 9, 2000 which is a continuation-in-part of U.S. Ser. No. 08/872,855 filed Jun. 11, 1997, which is a continuation-in-part of U.S. Ser. No. 08/832,633 filed Apr. 4, 1997, abandoned, the entire contents of each of which is incorporated herein by reference.

### 1. BACKGROUND OF THE INVENTION

Notch, first identified in Drosophila, is the founding member of a family of transmembrane receptor proteins that mediate cell responses to intrinsic and/or extrinsic developmental cues. The cellular response to Notch signaling can be differentiation, proliferation and/or apoptosis depending on the specific developmental program. In addition to its role as a signal-transducing cell surface protein, Notch can exert its function by directly regulating gene transcription. The Notch signaling pathway comprises Notch proteins: Drosophila Notch, LIN-12 and GLP-1 in C. elegans and Notch 1-4 in mammals; ligands: Delta, Delta-1, Delta-like 1 and 3, Jagged 1 and Jagged 2 (Serrate 1 and 2 in Drosophila, respectively); intracellular effectors: CBF-1, Deltex and NF-kappa B; target genes: HES, bHLH and TLE; processing molecules: Kuzbanian; and modifiers: lunatic fringe, manic fringe, radical fringe, numb, numb-like and disheveled 1,2 3.

Structural conservation of Notch family members and their ligands are seen throughout phylogeny suggesting a conserved role for this signaling pathway in various species. The product of the Delta gene, acting as a ligand, and that of the Notch gene, acting as a receptor, are key components in a lateral-inhibition signaling pathway that regulates the detailed patterning of many different tissues in Drosophila (Bray (1998) Semin Cell Dev Biol 9:591). In humans, it has recently been shown that the Notch3 gene, located on chromosome 19, is mutated in CADASIL (for cerebral autosomal dominant arteriopathy with subcortical infarcts and leukoencephalopathy) patients (Joutel et al., (1996) Nature 383: 707-710). CADASIL causes a type of stroke and dementia whose key features include recurrent subcortical ischemic events, progressive vascular dementia, craniofacial paralysis, migraine and mood disorders with severe depression (Chabriat et al. (1995) Lancet 346: 934-939).

Defects in the Notch signaling pathway may also be involved in other neurological diseases. For example, the genes encoding the amyloid precursor proteins, presenilin 1 Notch family receptor in C. elegans, and contain mutations which have been linked to Alzheimer's (Levitan and Greenwald (1995) Nature 377:351-354). Therefore, the Notch signaling pathway may be required for proper neurologic development and function.

In addition, analysis of gene expression patterns for Notch and its ligands has indicated that Notch signaling may have a role in hematopoiesis (Milner and Bigas (1999) Blood 93:2431). Notch activation can lead to expansion of early progenitor cells resident in the bone marrow and other sites of hematopoiesis (Milner et al. (1994) Blood 83:2057). Notch-1 was also shown to be involved in determination of T-cell education and fate in the thymus (Robey (1999) An Rev Immunol 17:283). Furthermore, a subset of human T-cell leukemia patients harbor a translocation involving the Notch 1 gene which results in a constitutively active Notch protein (Ellisen et al. (1991) Cell 66:649). Truncated Notch 2 sequences have been thought to play a role in the development of thymomas in cats infected with the feline leukemia virus (Rohn et al. (1996) J Virol 70:8071). Compelling evidence that the Notch signaling pathway is involved in B-cell development is seen in B-cell malignancies induced by Epstein-Barr virus (EBV). EBNA2, the transforming protein of EBV, transactivates cellular genes by direct interaction with a primary component of the Notch pathway (Henkel et al. (1994) Science 265:92).

The Notch signaling pathway, specifically Notch family members and their ligands, therefore, have been implicated in a number of disease states including, for example, neurologic, vascular, and hematologic disorders, as well as various malignancies. Thus, Notch signaling pathway therapeutics are highly desirable for treating various diseases and disorders.

In developed countries, stroke is the third leading cause of death and the primary cause of acquired physical or cognitive impairment. Vascular dementia is the second leading cause of dementia, after Alzheimer's disease. CADASIL causes a type of stroke and dementia whose key features include recurrent subcortical ischemic events, progressive vascular dementia, craniofacial paralysis, migraine and mood disorders with severe depression (Chabriat et al., (1995) Lancet 346: 934-939). These symptoms usually start to appear at about 45 years of age, and patients typically die by 65. The condition is believed to be largely undiagnosed and therefore the prevalence is not precisely known.

CADASIL is associated with diffuse white-matter abnormalities on neuroimaging (Tournier-Lasserve et. al., (1991) Stroke 22:1297-1302). Pathological examination reveals multiple small, deep cerebral infarcts, a leukoencephalopathy and a non-atherosclerotic, non-amyloid angiopathy involving mainly the small cerebral arteries.(Baudrimont et al., (1993) Stroke 24: 122-125). Severe alterations of vascular smooth muscle cells are evident on ultrastructural analysis (Ruchoux et al., (1995) Acta. Neuropathol. 89:500-512).

It has recently been shown that the human Notch3 gene, located on chromosome 19, is mutated in CADASIL patients (Joutel et al., (1996) Nature 383: 707-710). Most of the mutations cause amino acid changes in the extracellular domain. Therefore, disruption of the Notch signaling pathway appears to be responsible for CADASIL stroke and dementia.

Defects in the Notch signaling pathway may also be involved in other neurological diseases, e.g., Alzheimer's disease. In fact, approximately 10% of cases of Alzheimer's disease are associated with mutations in genes encoding the amyloid precursor proteins, presenilin 1 (PS1) and presenilin 2 (PS2). About 25% of early-onset familial Alzheimer's cases are associated with a mutation in PS1. PS1 and PS2 are transmembrane proteins which are homologous to the C. elegans protein encoded by the sel-12 gene, which has been shown to be genetically linked to the C. elegans lin-12 gene, which encodes a Notch-family receptor (Levitan and Greenwald (1995) Nature 377:351-354; PS1 and PS2 are further described in PCT Publication No. WO 96/34099 (Oct. 31, 1996); Sel12 is further described in PCT Publication No. WO 97/11956) (Apr. 3, 1997). Furthermore, targeted disruption of PS 1 in mice results in reduced expression of mRNA encoding Notch 1 and Delta-like gene 1 (D111), a vertebrate Notch ligand, in the presomitic mesoderm compared to control mice (Wong et al. (1997) Nature 387:288). This indicates that PS1 is required for the spatiotemporal expression of genes involved in the Notch signaling pathway.

The Notch signaling pathway comprises Notch proteins, which are membrane proteins, and proteins interacting with Notch proteins, termed Delta proteins. The product of the Delta gene, acting as a ligand, and that of the Notch gene, acting as a receptor, are key components in a lateral-inhibition signaling pathway that regulates the detailed patterning of many different tissues in Drosophila (Vassin et al., (1987) EMBO J 6:3431-3440; Kopczynski et al., (1988) Genes Dev. 2:1723-1735; Fehon et al., (1990) Cell 61:523-534; Artavanis-Tsakonas et al., (1991) Trends, Genet. Sci. 7:403-407; Heitzler et al., (1991) Cell 64: 1083-1092; Greenwald et al., (1992) Cell 68: 271-281; Fortini et al., (1993) Cell 75: 124501247; and Muskavitch (1994) Devl. Biol. 166:415-430). During neurogenesis in particular, neural precursors, by expressing Delta, inhibit neighboring Notch-expressing cells from becoming committed to a neural fate. Mutations leading to a failure of lateral inhibition cause an overproduction of neurons, giving rise to a phenotype termed the “neurogenic phenotype” in Drosophila. For example, loss of Notch1 leads to somite defects and embryonic death in mice, whereas constitutively active mutant forms of Notch1 appear to inhibit cell differentiation in Xenopus and in cultured mammalian cells (Swiatek et al. (1994) Genes Dev. 8:707; Conlon et al. (1995) J. Development 121:1533; Lopan et al. (1994) Development 120:2385; and Nye et al. (1994) Development 120:2421). Similarly, reduced activity of X-Delta1 causes more cells to become primary neurons, whereas raised activity causes fewer cells to become primary neurons (Chitnis et al. (1995) Nature 375:761). Furthermore, loss of D111 function in mice leads to excessive neuronal differentiation, resulting in severe patterning defects in the paraxial mesoderm and a hyperplastic central nervous system (CNS) (Hrabe de Angelis et al. (1997) Nature 386:717). Thus, the Notch signaling pathway, in particular Delta proteins, mediate lateral inhibition during neurogenesis so that only a limited proportion of cells having the potential to become neurons will in fact differentiate into neurons.

The Notch family of proteins are transmembrane receptors containing several conserved peptide motifs. The extracellular domains contain many tandemly repeated copies of an epidermal growth factor (EGF) like motif. The intracellular domains contain six copies of another conserved motif, termed the Cdc10/ankyrin repeat. Both the EGF and the ankyrin-repeat motifs are found in many different proteins and, in at least some cases, they have been shown to be involved in protein-protein interactions.

The Drosophila Notch protein encodes a glycosylated transmembrane receptor having a molecular mass 350 KD, which is involved in cell-fate specification during development (Wharton et al., (1985) Cell 43:567-581; Artavanis-Tsakonas et al., (1995) Science 268: 225-232). Based on analysis of Drosophila mutants, it is thought that Notch is a receptor having different functional domains, with the intracellular domain having the intrinsic signal-transducing activity of the intact protein and the extracellular domain a regulating activity (Rebay et al., (1993) Cell 74: 319-329).

Several Notch orthologs and homologs have been identified in vertebrates (Larson et al., (1994) Genomics 24: 253-258). Furthermore, three Notch gene products (Notch1, also called TAN1, Notch2, and Notch3) have been characterized in humans (Ellisen et al., (1991) Cell 66:649-661; Stifani et al., (1992) Nature Genet. 2: 119-127). Notch1 gene translocations have been associated with a minority of T-cell lymphoblastic leukemias (Aster (1994) Cold Spring Harb. Symp. Quant. Biol. 59:125-136) and Notch3 has been linked with CADASIL.

A protein interacting with Notch was first discovered in Drosophila and has been called Delta protein. This protein encodes a transmembrane protein ligand, which contains tandem arrays of epidermal growth factor-like repeats in the extracellular domain. The Delta and Notch proteins can directly bind to each other and specific EGF-like domains are sufficient and necessary for this binding (Fehon et al., (1990) Cell 61:523-534; Rebay et al., (1991) Cell 67:687-699; and Lieber et al., (1992) Neuron 9: 847-859).

It is also possible that soluble forms of the protein also exist. Such soluble isoforms can arise through variable splicing of the Delta3 gene or alternatively as a result of proteolysis of a membranous isoform. In fact, a splice variant of a chicken Delta protein have been described in PCT Publication No. WO 97/01571 (Jan. 16, 1997). Furthermore, the human Delta-like polypeptide Dlk is a soluble protein (Jansen et al. (1994) Eur. J. Biochem. 225:83-92).

In addition to the Drosophila Delta protein, a chick Delta ortholog, C-Delta protein (Henrique et al., (1995) Nature 375: 787-790 and GenBank Accession No. U26590) two Xenopus orthologs, X-Delta-1 and X-Delta-2 (Chitnis et al., (1995) Nature 375:761-766 and GenBank Accession Nos. L42229 and U70843), a mouse ortholog (GenBank Accession No. X80903), a delta-like human gene 1(Dlk) (Bettenhausen et al., (1995) Development 121:2407-2418) a rat ortholog (GenBank Accession No. U78889), and a Zebrafish ortholog (GenBank Accession No. Y11760) have been identified. Xenopus, chick and mouse Delta genes are also disclosed in PCT Publication No. WO 97/01571 (Jan. 16, 1997). The patent application also discloses a partial and error prone human Delta homolog (hD1). Nucleotide sequence of human Notch genes are disclosed in PCT Publication No. WO 92/19734 (Nov. 12, 1992) and PCT Publication No. WO 94/07474 (Apr. 14, 1994).

Notch signaling pathway therapeutics, in particular Delta therapeutics are highly desirable for treating various diseases and disorders, including neurological and vascular disorders.

### 2. SUMMARY OF THE INVENTION

The invention is based at least in part on the discovery of a human gene encoding a novel Delta protein, and its mouse homolog, each of which differs substantially from the previously described Delta proteins. Accordingly, the novel Delta proteins of the invention are referred to herein as Delta3 proteins. Thus, the invention provides Delta3 proteins, and nucleic acids encoding Delta3 proteins. An exemplary human Delta3 (hDelta3) is contained in a plasmid which was deposited with the ATCC® on Mar. 5, 1997, and has been assigned ATCC® accession number 98348.

Based on Northern blot analysis of RNA prepared from a number of human tissues, a 3.5 kb message was expressed in fetal brain, lung, liver and kidney; and adult heart, placenta, lung, skeletal muscle, kidney, pancreas, spleen, thymus, prostate, testis, ovary, small intestine and colon. In addition, the hDelta3 gene was found to be expressed at relatively high levels in at least some tumor cells (e.g., colon carcinoma) and its expression can be up-regulated in response to various growth factors (e.g., bFGF and VEGF). Furthermore, the expression of hDelta3 was also shown to increase in response to a signal-induced differentiation of endothelial cells, indicating a role for hDelta3 in modulating the growth and/or differentiation of cells, in particular endothelial cells.

In situ hybridization of a panel of adult and embryonic tissues using a probe complementary to mRNA of mDelta3 showed that mDelta3 expression was most abundant and widespread during embryogenesis. Strongest expression was observed in the eye during all stages of embryogenesis tested, mDelta3 was also seen in the developing lung, thymus and brown fat. In addition to the focal expression seen above, a multi-focal, scattered pattern was seen throughout the embryo. This signal pattern was more focused in the cortical region of the kidney and outlining the intestinal tract. Adult expression was highest in the ovary and the cortical regions of the kidney and adrenal gland. The expression seen during embryogenesis indicates that Delta3 has a role in cell growth and/or differentiation.

As demonstrated herein, Delta3 encodes a Notch lignad. In particular, data presented herein demonstrates that hDelta3 encodes a Notch ligand, as it has been shown to block the differentiation of C2C12 into myotubes in a similar fashion to other Notch ligands (e.g., Jagged 1).

As described herein, the hDelta3 gene has been localized by Southern blotting a membrane containing DNA from a panel of a human/hamster mono-chromosomal somatic cell hybrids. The results demonstrate that human Delta3 is located on human chromosome 15, close to framework markers D15S1244 and D15S144, a chromosomal region that has been associated with the neurological disease Agenesis of the Corpus Callosum with Peripheral Neuropathy (ACCPN) (Casaubon et al. (1996) Am J. Hum. Genet. 58:28). Accordingly, polymorphisms in Delta3 are thought to be involved in this neurological disease. As described further herein, Delta3 is also likely to be involved in other neurological diseases as well as in non-neurological diseases.

In one aspect, the invention features isolated Delta3 nucleic acid molecules, e.g., mammalian, such as human or mouse, Delta3 nucleic acids. The disclosed molecules can be non-coding, (e.g., probe, antisense or ribozyme molecules) or can encode a functional Delta3 polypeptide, e.g., a polypeptide which can modulate at least one activity of a Delta3 polypeptide. In one embodiment, the nucleic acid molecules hybridize to the Delta3 gene contained in the plasmid having American Type Culture Collection (ATCC®) Accession Number 98348. In another embodiment, the claimed nucleic acid is capable of hybridizing under stringent conditions to the nucleotide sequence set forth in SEQ ID NOS: 1, 3, 24, 26, 27, 29, 31, 33, 35, 37, 39, 41, 43 or 45, or the nucleotide sequence of the cDNA of a clone deposited with the ATCC® as Accession Number 98348, or to the complement thereof.

In further embodiments, the nucleic acid molecule is a Delta3 nucleic acid molecule that is at least about 50%, 55%, 60%, 70%, preferably 80%, more preferably 85%, and even more preferably at least about 95% or 98% identical to the nucleic acids shown in SEQ ID Nos: 1, 3, 24, 26, 27, 29, 31, 33, 35, 37, 39, 41, 43 or 45, or the nucleotide sequence of the cDNA of a clone deposited with the ATCC® as Accession Number 98348, or a complement thereof.

In another embodiment, the nucleic acid molecule is a Delta3 nucleic acid that is at least about 50%, 55%, 60%, 65%, 70%, preferably 80%, more preferably 85%, and even more preferably at least about 95% or 98% identical to the nucleic acids shown in SEQ ID NOS: 1, 3, 24, 26, 27, 29, 31, 33, 35, 37, 39, 41, 43 or 45, or the nucleotide sequence of the cDNA of a clone deposited with the ATCC® as Accession Number 98348 or a complement thereof, wherein such nucleic acid molecules encode polypeptides or proteins that exhibit at least one structural and/or functional feature of a polypeptide of the invention.

In another embodiment, the nucleic acid molecule encodes a polypeptide that is at least about 55%, 60%, 70%, preferably 80%, more preferably 85%, and even more preferably at least about 90, 95% or 98% identical to the polypeptide shown in SEQ ID NOS: 2, 25, 28, 30, 32, 34, 36, 38, 40, 42, 44 or 46, or the amino acid sequence encoded by the cDNA of a clone deposited with the ATCC® as Accession Number 98348, or a complement thereof.

In another embodiment, the nucleic acid molecule encodes a polypeptide that is at least about 55%, 60%, 70%, preferably 80%, more preferably 85%, and even more preferably at least about 90, 95% or 98% identical to the polypeptide shown in SEQ ID NOS: 2, 25, 28, 30, 32, 34, 36, 38, 40, 42, 44 or 46, or the amino acid sequence encoded by the cDNA of a clone deposited with the ATCC® as Accession Number 98348, or a complement thereof, wherein such nucleic acid molecules encode polypeptides or proteins that exhibit at least one structural and/or functional feature of a polypeptide of the invention.

In another embodiment, the nucleic acid molecule encodes a polypeptide that is at least about 72%, preferably 80%, more preferably 85%, and even more preferably at least about 90 or 95% similar to the polypeptide shown in SEQ ID NOS: 2, 25, 28, 30, 32, 34, 36, 38, 40, 42, 44 or 46, or the hDelta 3 cDNA sequence contained in the plasmid having ATCC® Accession Number 98348, or a complement thereof.

In another embodiment, the nucleic acid molecule encodes a polypeptide that is at least about 72%, preferably 80%, more preferably 85%, and even more preferably at least about 90 or 95% similar to the polypeptide shown in SEQ ID NOS: 2, 25, 28, 30, 32, 34, 36, 38, 40, 42, 44 or 46, or the Delta 3 cDNA sequence contained in the plasmid having ATCC® Accession Number 98348, or a complement thereof, wherein such nucleic acid molecules encode polypeptides or proteins that exhibit at least one structural and/or functional feature of a polypeptide of the invention.

Also within the invention are nucleic acid molecules which encode a fragment of a polypeptide having the amino acid sequence of any of SEQ ID NOS: 2, 25, 28, 30, 32, 34, 36, 38, 40, 42, 44 or 46, the fragment including at least 15 (20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 440, 460, 475, 500, 525, 550, 575, 600, 625, 650, 675, or 685) contiguous amino acids of any of SEQ ID NOS: 2, 25, 28, 30, 32, 34, 36, 38, 40, 42, 44 or 46, or the polypeptide encoded by the cDNA of a clone deposited with the ATCC® as Accession Number 98348.

Also within the invention are nucleic acid molecules which encode a fragment of a polypeptide having the amino acid sequence of any of SEQ ID NOS: 2, 25, 28, 30, 32, 34, 36, 38, 40, 42, 44 or 46, the fragment including at least 15 (20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 440, 460, 475, 500, 525, 550, 575, 600, 625, 650, 675, or 685) contiguous amino acids of any of SEQ ID NOS: 2, 25, 28, 30, 32, 34, 36, 38, 40, 42, 44 or 46, or the polypeptide encoded by the cDNA of a clone deposited with the ATCC® as Accession Number 98348, wherein the fragment exhibits at least one structural and/or functional feature of a polypeptide of the invention.

The nucleic acids of the invention can comprise a nucleotide sequence encoding at least one domain or motif of a Delta3 protein, i.e., a domain or motif selected from the group consisting of an amino-terminal signal peptide, a Delta-Serrated lag-2 (DSL) domain, Epidermal Growth Factor (EGF)-like domain 1, EGF-like domain 2, EGF-like domain 3, EGF-like domain 4, EGF-like domain 5, EGF-like domain 6, EGF-like domain 7, EGF- like domain 8, a Delta3 extracellular domain, a transmembrane domain (TM), and a cytoplasmic domain. Other nucleic acids of the invention encode soluble Delta3 proteins, e.g., Delta3 proteins comprising at least a portion, such as one or more domains, of the extracellular domain of a Delta3 protein. A soluble Delta3 protein is a protein that is soluble at standard physiological conditions, and includes, but is not limited to a Delta3 protein that does not comprise a transmembrane domain, e.g., an extracellular Delta3 domain. These soluble polypeptides may or may not comprise a signal peptide. Other such nucleic acids encode soluble fusion proteins comprising Delta3 amino acid sequence and a heterologous amino acid sequence, e.g., an immunoglobulin constant region.

The invention also provides probes and primers comprising substantially purified oligonucleotides, which correspond to a region of nucleotide sequence which hybridizes to at least about 6 consecutive nucleotides of the sequences set forth as SEQ ID Nos: 1, 3, 24, 26, 27, 29, 31, 33, 35, 37, 39, 41, 43 or 45, or the nucleotide sequence of the cDNA of a clone deposited with the ATCC® as Accession Number 98348, or a complement thereof, or naturally-occurring mutants thereof. In preferred embodiments, the probe/primer further includes a label group attached thereto, which is capable of being detected.

The invention features nucleic acid molecules of at least 425, 450, 500, 550, 600, 650, 700, 750, 800, 850, 900, 950, 1000, 1100, 1200, 1300, 1400, 1500, 1600, 1700, 1800, 1900, 2000, 2100, 2200, 2300, 2400, 2500, 2600, 2700, or 2800 nucleotides of the nucleotide sequence of SEQ ID NO: 1, the nucleotide sequence of the human Delta3 cDNA clone of ATCC® Accession NO: 98348, or a complement thereof. The invention also features nucleic acid molecules comprising at least 25, 50, 100, 150, 200, 250, 300, 350, 400, 450, 500, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050 or 2061 nucleotides of nucleic acids 1 to 2062 of SEQ ID NO: 1, or a complement thereof, wherein such nucleic acid molecules encode polypeptides or proteins that exhibit at least one structural and/or functional feature of a polypeptide of the invention.

The invention features nucleic acid molecules which include a fragment of at least 340, 400, 450, 500, 550, 600, 650, 700, 750, 800, 850, 900, 950, 1000, 1050, 1100, 1150, 1200, 1250, 1300, 1350, 1400, 1450, 1450, 1500, 1550, 1600, 1650, 1700, 1750, 1800, 1850, 1900, 1950, 2000, 2050 or 2058 nucleotides of the nucleotide sequence of SEQ ID NO: 3, or a complement thereof. The invention also features nucleic acid molecules comprising at least 25, 50, 100, 150, 200, 250, 300, 350, 400, 450, 500, 600, 650, 700, 750, 800, 850, 900, 950, 1000, 1050, 1100, 1150, 1200, 1250, 1300, 1350, 1400, 1450, 1500, 1550, 1600, 1650, 1700, or 1724 nucleotides of nucleic acids 1 to 1725 of SEQ ID NO: 3, or a complement thereof.

The invention features nucleic acid molecules of at least 480, 500, 550, 600, 650, 700, 750, 800, 850, 900, 950, 1000, 1050, 1100, 1150, 1200, 1250, 1300, 1350, 1400, 1450, 1450, 1500, 1550, 1600, 1650, 1700, 1750, 1800, 1850, 1900, 1950, 2000, 2050, 2100, 2200, 2300, 2400, 2500, 2600, 2700, 2800, 2900, 3000, 3100 or 3130 nucleotides of the nucleotide sequence of SEQ ID NO: 24, the nucleotide sequence of the mouse Delta3 cDNA, or a complement thereof. The invention also features nucleic acid molecules comprising at least 25, 50, 100, 150, 200, 250, 300, 350, 400, 450, 500, 600, 650, 700, 750, 800, 850, 900, 950, 1000, 1050, 1100, 1150, 1200, 1250, 1300, 1350, 1400, 1450, 1500, or 1529 nucleotides of nucleic acids 1 to 1530 of SEQ ID NO: 24, or a complement thereof, wherein such nucleic acid molecules encode polypeptides or proteins that exhibit at least one structural and/or functional feature of a polypeptide of the invention.

The invention features nucleic acid molecules which include a fragment of at least 415, 450, 500, 550, 600, 650, 700, 750, 800, 850, 900, 950, 1000, 1050, 1100, 1150, 1200, 1250, 1300, 1350, 1400, 1450, 1450, 1500, 1550, 1600, 1650, 1700, 1750, 1800, 1850, 1900, 1950, 2000, 2050, 2100 nucleotides of the nucleotide sequence of SEQ ID NO: 26, or a complement thereof. The invention also features nucleic acid molecules comprising at least 25, 50, 100, 150, 200, 250, 300, 350, 400, 450, 500, 600, 650, 700, 750, 800, 850, 900, 950, 1000, 1050, 1100, 1150, 1200, 1250, 1300, 1350, 1400, 1450, 1500 or 1529 nucleotides of nucleic acids 1 to 1530 of SEQ ID NO: 3, or a complement thereof.

Another aspect of the invention provides vectors, e.g., recombinant expression vectors, comprising a nucleic acid molecule of the invention. In another embodiment the invention provides host cells containing such a vector. The invention also provides methods for producing a polypeptide of the invention by culturing, in a suitable medium, a host cell of the invention containing a recombinant expression vector encoding a polypeptide of the invention such that the polypeptide of the invention is produced.

For expression, the subject Delta3 nucleic acids can include a mammalian transcriptional regulatory sequence, e.g., at least one of a transcriptional promoter (e.g., for constitutive expression or inducible expression) or transcriptional enhancer sequence, the regulatory sequence is operably linked to the Delta3 gene sequence. Such regulatory sequences in conjunction with Delta3 nucleic acid molecules can be useful in vectors for gene expression. This invention also describes host cells transfected with said expression vectors whether prokaryotic or eukaryotic, also in vitro (e.g., cell culture) and in vivo (e.g., transgenic) methods for producing Delta3 proteins by employing said expression vectors. In a preferred embodiment, the Delta3 nucleic acids are cloned into a mammalian expression vector, and transfected into mammalian cells. The use of mammalian cells increases the likelihood of proper protein folding and post-translational modification of the expressed proteins.

The invention also features nucleic acid molecules that hybridize under stringent conditions to a nucleic acid molecule comprising the nucleotide sequence of any of SEQ ID NOS: 1, 3, 24, 26, 27, 29, 31, 33, 35, 37, 39, 41, 43 or 45 of the cDNA of a clone deposited with the ATCC® as Accession Number 98348, or a complement thereof, wherein preferably such nucleic acid molecules encode polypeptides or proteins that exhibit at least one structural and/or functional feature of a polypeptide of the invention. In other embodiments, the nucleic acid molecules are at least 485 (500, 550, 600, 650, 700, 800, 900, 1000, 1100, 1200, 1300, 1400, 1500, 1600, 1700, 1800, 1900, 2000, 2100, 2200, 2300, 2400, 2500, 2600, 2700, or 2800) nucleotides in length and hybridize under stringent conditions to a nucleic acid molecule comprising the nucleotide sequence of any of SEQ ID NOS: 1, 3, 24, 26, 27, 29, 31, 33, 35, 37, 39, 41, 43 or 45 of the cDNA of a clone deposited with the ATCC® as Accession Number 98348, or a complement thereof.

In preferred embodiments, the isolated nucleic acid molecules encode a cytoplasmic, transmembrane, or extracellular domain of a polypeptide of the invention.

The invention includes nucleic acid molecules which encode an allelic variant of a polypeptide comprising the amino acid sequence of any of SEQ ID NOS: 2, 25, 28, 30, 32, 34, 36, 38, 40, 42, 44 or 46, or an amino acid sequence encoded by the cDNA of a clone deposited with the ATCC® as Accession Number 98348, or a complement thereof, wherein the nucleic acid molecule hybridizes under stringent conditions to a nucleic acid molecule having a nucleic acid sequence encoding any of SEQ ID NOS:1, 3, 24, 26, 27, 29, 31, 33, 35, 37, 39, 41, 43 or 45, or the nucleotide sequence of the cDNA of a clone deposited with the ATCC® as Accession Number 98348, or a complement thereof.

The invention includes nucleic acid molecules which encode an allelic variant of a polypeptide comprising the amino acid sequence of any of SEQ ID NOS: 2, 25, 28, 30, 32, 34, 36, 38, 40, 42, 44 or 46, or an amino acid sequence encoded by the cDNA of a clone deposited with the ATCC® as Accession Number 98348, or a complement thereof, wherein the nucleic acid molecule hybridizes under stringent conditions to a nucleic acid molecule having a nucleic acid sequence encoding a polypeptide having any of the amino acid sequences shown in SEQ ID NOS: 2, 25, 28, 30, 32, 34, 36, 38, 40, 42, 44 or 46, or the amino acid sequence encoded by the cDNA of a clone deposited with the ATCC® as Accession Number 98348, or a complement thereof, wherein such nucleic acid molecules encode polypeptides or proteins that exhibit at least one structural and/or functional feature of a polypeptide of the invention.

In one embodiment of a nucleotide sequence of human Delta3, the nucleotide at position 455 is a guanine (G)(SEQ ID NO: 1). In this embodiment, the amino acid at position 40 is glutamate (E)(SEQ ID NO: 2). In another embodiment of a nucleotide sequence of human Delta3, the nucleotide at position 455 is a cytosine (C)(SEQ ID NO: 27). In this embodiment, the amino acid at position 40 is glutamine (Q)(SEQ ID NO: 28). In another embodiment of a nucleotide sequence of human Delta3, the nucleotide at position 455 is a thymidine (T)(SEQ ID NO: 29). In this embodiment, the amino acid at position 40 is a stop codon (SEQ ID NO: 30). In another embodiment of a nucleotide sequence of human Delta3, the nucleotide at position 455 is a adenine (A)(SEQ ID NO: 31). In this embodiment, the amino acid at position 40 is lysine (K)(SEQ ID NO: 32).

In another embodiment, the invention provides an isolated nucleic acid molecule which is antisense to the coding strand of a nucleic acid of the invention.

In another aspect, the invention features isolated Delta3 polypeptides, preferably substantially pure preparations, e.g., of plasma-purified or recombinantly produced Delta3 polypeptides. Preferred proteins and polypeptides possess at least one biological activity of the corresponding naturally-occurring human polypeptide. Such an activity can be a direct activity, such as an association with or an enzymatic activity on a second protein or an indirect activity, such as a cellular signaling activity mediated by interaction of the protein with a second protein. Thus, such activities include, for example, ones related to Delta3's function as a Notch ligand. Delta3 activities include, e.g., (1) the ability to form protein-protein interactions with proteins in the signaling pathway of the naturally-occurring polypeptide; (2) the ability to bind a ligand of the naturally-occurring polypeptide; (3) the ability to bind to an intracellular target of the naturally-occurring polypeptide; (4) the ability to modulate cellular proliferation; (5) the ability to modulate cellular differentiation; (6) the ability to modulate chemotaxis and/or migration; and/or (7) the ability to modulate cell death; (8) maintenance of energy homeostasis (e.g., regulating the balance or imbalance between energy storage and energy expenditure, for example, increasing or decreasing energy expenditure; (9) regulation of adaptive thermogenesis (e.g., regulation of the biogenesis of mitochondria, regulation of the expresion of mitochondrial enzymes, regulation of expression of uncoupling proteins; (10) regulation of adiposity; (11) modulation of the efficiency of energy storage; (12) regulation of appetite; (13) expansion/reduction of fat mass; (14) regulation of vasculogenesis (blood vessel formation); (15) regulation of tumor angiogenesis; (16) regulation of wound healing; (17) expansion/reduction of tumor mass; (18) the ability to modulate development, differentiation, proliferation and/or activity of immune cells (e.g., leukocytes and macrophages), endothelial cells and smooth muscle cells; (19) the ability to modulate the host immune response; (20) the ability to modulate the development of organs, tissues and/or cells of the embryo and/or fetus; (21) the ability to modulate cell-cell interactions and/or cell-extracellular matrix interactions; (22) the ability to modulate atherosclerosis, e.g., the initiation and progression of atherosclerosis; (23) the ability to modulate atherogenesis; (24) the ability to modulate inflammatory functions e.g., by modulating leukocyte adhesion to extracellular matrix and/or endothelial cells; (25) the ability to form, e.g., stabilize, promote, facilitate, inhibit, or disrupt, cell to cell and cell to blood product interaction, e.g., between leukocytes and platelets or leukocytes and vascular endothelial cells; (26) ability to modulate development, differentiation and activity of skeletal muscle cells and tissue; and (27) ability to act in stem cell preservation.

In certain embodiments, the subject polypeptides are capable of modulating an activity of a Delta3 polypeptide, e.g., cell growth and/or differentiation or induction of apoptosis. In other embodiments, the subject Delta3 polypeptides can modulate neurogenesis (e.g., by inhibiting Notch expressing cells from becoming committed to a neural fate). In addition, Delta3 polypeptides which specifically antagonize the activity of a native Delta3 polypeptide, such as may be provided by truncation mutants or other dominant negative mutants, are also specifically provided herein.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity.

In one embodiment, the polypeptide is identical to a Delta3 protein represented in SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Preferably, a Delta3 polypeptide has an amino acid sequence, which is at least about 55%, 60%, 70%, preferably at least about 80%, more preferably at least about 90%, and even more preferably at least about 95% or 98% identical to the polypeptide represented by SEQ ID NOs: 2, 25, 28, 30, 32, 34, 36, 38, 40, 42, 44 or 46, or the amino acid sequence encoded by the cDNA of a clone deposited with the ATCC® as Accession Number 98348.

Also within the invention are isolated polypeptides or proteins having an amino acid sequence that is at least about 55%, preferably 65%, 75%, 85%, 95%, or 98% identical to the amino acid sequence of any of SEQ ID NOs: 2, 25, 28, 30, 32, 34, 36, 38, 40, 42, 44 or 46, or the amino aicd sequence encoded by the cDNA of a clone deposited with the ATCC® as Accession Number 98348, wherein the polypeptides or proteins also exhibit at least one structural and/or functional feature of a polypeptide of the invention.

Also within the invention are isolated polypeptides or proteins which preferably are encoded by a nucleic acid molecule having a nucleotide sequence that is at least about 55%, more preferably 60%, 65%, 75%, 85%, or 95% identical the nucleic acid sequence encoding any of SEQ ID NOs: 2, 25, 28, 30, 32, 34, 36, 38, 40, 42, 44 or 46, wherein the polypeptides or proteins preferably also exhibit at least one structural and/or functional feature of a polypeptide of the invention, and isolated polypeptides or proteins which are encoded by a nucleic acid molecule having a nucleotide sequence which hybridizes under stringent hybridization conditions to a nucleic acid molecule having the sequence of any of SEQ ID NOs:1, 3, 24, 26, 27, 29, 31, 33, 35, 37, 39, 41, 43 or 45, or a complement thereof, or the non-coding strand of the cDNA of a clone deposited with the ATCC® as Accession Number 98348.

In a preferred embodiment, the Delta3 polypeptide is encoded by a nucleic acid which hybridizes in high stringency conditions with a nucleic acid sequence represented in one of SEQ ID NOs: 1, 3, 24, 26, 27, 29, 31, 33, 35, 37, 39, 41, 43 or 45 or with the nucleic acid contained in the plasmid having ATCC® Accession NO: 98348.

The subject Delta3 proteins also include modified proteins, which are resistant to post-translational modification, as for example, due to mutations which alter modification sites (such as tyrosine, threonine, serine or asparagine residues), or which prevent glycosylation of the protein, or which prevent interaction of the protein with intracellular proteins involved in signal transduction.

The Delta3 polypeptide can comprise a full-length protein, such as represented in SEQ ID NO: 2, 25, 28, 30, 32, 34, 36, 38, 40, 42, 44 or 46, or it can comprise a fragment corresponding to one or more particular motifs/domains (e.g., an extracellular domain, a DSL domain or an EGF-like domain, all of which are described below), or to other sizes, e.g., at least 5, 10, 25, 50, 100, 150, 200, 250, 300, 350, 400, 450, 500, 550, 600 or 650 amino acids in length.

The polypeptides of the invention can comprise at least one domain or motif of a Delta3 protein, i.e., a domain or motif selected from the group consisting of an amino-terminal signal peptide, a Delta-Serrated lag-2 (DSL) domain, Epidermal Growth Factor (EGF)-like domain 1, EGF-like domain 2, EGF-like domain 3, EGF-like domain 4, EGF-like domain 5, EGF-like domain 6, EGF-like domain 7, EGF-like domain 8, a Delta3 extracellular domain, a transmembrane domain (TM), and a cytoplasmic domain. Other polypeptides comprise soluble Delta3 proteins, e.g., Delta3 proteins comprising at least a portion, such as one or more domains, of the extracellular domain of a Delta3 protein. A soluble Delta3 protein is a protein that is soluble at physiological conditions, and includes but is not limited to a Delta3 protein that does not comprise a transmembrane domain, e.g., an extracellular Delta3 domain. These soluble polypeptides may or may not comprise a signal peptide. Other such polypeptides comprise soluble fusion proteins comprising Delta3 amino acid sequence and a heterologous amino acid sequence, e.g., an immunoglobulin constant region.

In one embodiment, the isolated polypeptide of the invention lacks both a transmembrane and a cytoplasmic domain. In another embodiment, the polypeptide lacks both a transmembrane domain and a cytoplasmic domain and is soluble under physiological conditions.

Also within the invention are polypeptides which are allelic variants of a polypeptide that includes the amino acid sequence of any of SEQ ID NOs: 2, 25, 28, 30, 32, 34, 36, 38, 40, 42, 44 or 46, or an amino acid sequence encoded by the cDNA of a clone deposited with the ATCC® as Accession Number 98348, wherein the polypeptide is encoded by a nucleic acid molecule which hybridizes under stringent conditions to a nucleic acid molecule having the sequence of any of SEQ ID Nos:1, 3, 24, 26, 27, 29, 31, 33, 35, 37, 39, 41, 43 or 45, or the nucleotide sequence of the cDNA of a clone deposited with the ATCC® as Accession Number 98348, or a complement thereof.

Also within the invention are polypeptides which are allelic variants of a polypeptide that includes the amino acid sequence of any of SEQ ID NOs: 2, 25, 28, 30, 32, 34, 36, 38, 40, 42, 44 or 46, or an amino acid sequence encoded by the cDNA of a clone deposited with the ATCC® as Accession Number 98348, wherein the polypeptide is encoded by a nucleic acid molecule which hybridizes under stringent conditions to a nucleic acid molecule having the sequence of any of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, or a complement thereof, wherein such nucleic acid molecules encode polypeptides or proteins that exhibit at least one structural and/or functional feature of a polypeptide of the invention.

Another aspect of the invention features fusion proteins comprising a Delta3 amino acid sequence. For instance, the Delta3 protein can be provided as a recombinant fusion protein which includes a second polypeptide portion, e.g., a second polypeptide having an amino acid sequence unrelated (heterologous) to the Delta3 polypeptide, (e.g., the second polypeptide portion is glutathione-S-transferase, an enzymatic sequence such as alkaline phosphatase or an epitope tag).

Fusion proteins of the invention include, for example, Delta3 immunoglobulin (Delta3-Ig) fusion proteins. For example, a Delta3 fusion protein can comprise the extracellular portion of a Delta3 protein fused to the constant region of an immunoglobulin molecule. Preferred extracellular portions comprise at least one domain selected from the group consisting of a signal peptide, a DSL domain, and the eight EGF-like domains of a Delta3 protein. An even more preferred extracellular domain comprises an amino acid sequence from amino acid 1 to amino acid 529 of SEQ ID NO: 2 or from amino acid 1 to 530 of SEQ ID NO: 25. Yet other preferred Delta3 fusion proteins comprise a portion of a Delta3 protein that is sufficient for binding to a second protein, such as the DSL domain to a second protein which is, for example, a Notch protein.

Yet another aspect of the present invention concerns an immunogen comprising a Delta3 polypeptide in an immunogenic preparation, the immunogen being capable of eliciting an immune response specific for a Delta3 polypeptide, e.g., a humoral response, an antibody response and/or cellular response. In preferred embodiments, the immunogen comprises an antigenic determinant, e.g., a unique determinant, from the protein represented by SEQ ID NOs: 2, 25, 28, 30, 32, 34, 36, 38, 40, 42, 44 or 46, or the amino acid sequence encoded by the cDNA of a clone deposited with the ATCC® as Accession Number 98348.

A still further aspect of the present invention features antibodies and antibody preparations specifically reactive with an epitope of the Delta3 protein. In preferred embodiments, the antibody specifically binds to an epitope of a polypeptide shown in SEQ ID NOs: 2, 25, 28, 30, 32, 34, 36, 38, 40, 42, 44 or 46, or the amino acid sequence encoded by the cDNA of a clone deposited with the ATCC® as Accession Number 98348.

In yet a further aspect, the invention provides substantially purified antibodies or fragments thereof, including human or non-human antibodies or fragments thereof, which antibodies or fragments specifically bind to a polypeptide comprising an amino acid sequence selected from the group consisting of: the amino acid sequence of SEQ ID NOs: 2, 25, 28, 30, 32, 34, 36, 38, 40, 42, 44 or 46, or the amino acid sequence encoded by the cDNA insert of the plasmid deposited with the ATCC® as Accession Number 98348; a fragment of at least 15 amino acid residues of the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348; an amino acid sequence which is at least 95% identical to the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348 wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, under conditions of hybridization of 6× SSC at 45□C. and washing in 0.2× SSC, 0.1% SDS at 65□C. In various embodiments, the substantially purified antibodies of the invention, or fragments thereof, can be human, non-human, chimeric and/or humanized antibodies.

In another aspect, the invention provides human or non-human antibodies or fragments thereof, which antibodies or fragments specifically bind to a polypeptide comprising an amino acid sequence selected from the group consisting of: the amino acid sequence of SEQ ID NOs: 2, 25, 28, 30, 32, 34, 36, 38, 40, 42, 44 or 46, or the amino acid sequence encoded by the cDNA insert of the plasmid deposited with the ATCC® as Accession Number 98348; a fragment of at least 15 amino acid residues of the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348; an amino acid sequence which is at least 95% identical to the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348 wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, under conditions of hybridization of 6× SSC at 45° C. and washing in 0.2× SSC, 0.1% SDS at 65° C. With respect to non-human antibodies, such antibodies can be goat, mouse, sheep, horse, chicken, rabbit, or rat antibodies. Alternatively, the non-human antibodies of the invention can be chimeric and/or humanized antibodies. In addition, the human and non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of: the amino acid sequence of SEQ ID NOs: 2, 25, 28, 30, 32, 34, 36, 38, 40, 42, 44 or 46, or the amino acid sequence encoded by the cDNA insert of the plasmid deposited with the ATCC® as Accession Number 98348; a fragment of at least 15 amino acid residues of the amino acid sequence of SEQ ID NOs: 2, 25, 28, 30, 32, 34, 36, 38, 40, 42, 44 or 46; an amino acid sequence which is at least 95% identical to the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, under conditions of hybridization of 6× SSC at 45° C. and washing in 0.2× SSC, 0.1% SDS at 65° C. The monoclonal antibodies can be human, humanized, chimeric and/or non-human antibodies.

In a particularly preferred embodiment, the substantially purified antibodies or fragments thereof, the human and non-human antibodies or fragments thereof, and/or monoclonal antibodies or fragments thereof, of the invention specifically bind to an extracellular domain of the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Preferably, the extracellular domain to which the antibody, or fragment thereof, binds comprises amino acid residues 1-529 of SEQ ID NO: 2 of human Delta3, or amino acid residues 1-530 of SEQ ID NO: 25 of murine Delta3.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention and instructions for use. Such kits can also comprise an antibody of the invention conjugated to a detectable substance and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

In addition, the polypeptides of the invention or biologically active portions thereof, or antibodies of the invention, can be incorporated into pharmaceutical compositions, which optionally include pharmaceutically acceptable carriers.

The invention also features transgenic non-human animals which include (and preferably express) a heterologous form of a Delta3 gene described herein, or which misexpress (e.g., do not express) an endogenous Delta3 gene (e.g., an animal in which expression of one or more of the subject Delta3 proteins is disrupted). Such a transgenic animal can serve as an animal model for studying cellular or tissue disorders comprising mutated or mis-expressed Delta3 alleles or for use in drug screening. For example, the transgenic animals of the invention can be used as an animal model to study a neurological disease, e.g., ACCPN. Alternatively, such a transgenic animal can be useful for expressing recombinant polypeptides, and for generating cells, e.g., cell lines that can, for example, be utilized as part of screening techniques.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention by measuring the expression of the polypeptide or nucleic acid in the presence and absence of the compound.

In one embodiment, the invention provides assays, e.g., for screening test compounds to identify agonists, or alternatively, antagonists, of a Delta3 activity. For example, the test compound may positively or negatively influence an interaction between a Delta3 protein and a Delta3 target molecule, for example, a Notch protein. An exemplary method includes the steps of (i) combining a Delta3 polypeptide or active fragment thereof, with a Delta3 target molecule, e.g., Notch, and a test compound, e.g., under conditions wherein, but for the test compound, the Delta3 protein and target molecule are able to interact; and (ii) detecting the formation of a complex which includes the Delta3 protein and the target molecule either by directly quantitating the complex, by measuring inductive effects of the Delta3 protein, or, in the instance of a substrate, measuring the conversion to product. A statistically significant change, such as a decrease, in the interaction of the Delta3 and target molecule in the presence of a test compound (relative to what is detected in the absence of the test compound) is indicative of a modulation (e.g., suppression or potentiation of the interaction between the Delta3 protein and the target molecule).

The invention provides yet other methods for identifying compounds which modulate a Delta activity. For example, a compound that interacts with a Delta3 protein can be identified by contacting a Delta3 protein with a test compound. Either the test compound or the Delta3 protein can be labeled. Optionally, the non-labeled compound or Delta3 protein can be attached to a solid phase support. Binding of the test compound to the Delta3 protein can then be determined, e.g by measuring the amount of label. Such a Delta3 binding molecule can be an agonist or an antagonist. In one embodiment, an agonist of a Delta3 activity is identified by contacting a cell having a Delta3 protein with a test compound and measuring a Delta3 activity, e.g., expression of a gene which is regulated by binding of a protein, e.g., a Notch protein, to Delta3. An increased expression of the gene when the cell is incubated with the test compound relative to incubation in the absence of the test compound indicates that the test compound is a Delta3 agonist. The gene that is monitored can also be a reporter gene transfected to a cell, the reporter gene being under the control of a promoter of a gene which is regulated by Delta3.

In another aspect, the invention provides methods for modulating activity of a polypeptide of the invention comprising contacting a cell with an agent that modulates (inhibits or stimulates) the activity or expression of a polypeptide of the invention such that activity or expression in the cell is modulated. In one embodiment, the agent is an antibody that specifically binds to a polypeptide of the invention.

In another embodiment, the agent modulates expression of a polypeptide of the invention by modulating transcription, splicing, or translation of an mRNA encoding a polypeptide of the invention. In yet another embodiment, the agent is a nucleic acid molecule having a nucleotide sequence that is antisense to the coding strand of an mRNA encoding a polypeptide of the invention.

Yet another aspect of the present invention concerns methods for treating diseases or conditions that are caused or contributed to by an aberrant Delta3 expression, level, or activity, e.g., aberrant cell proliferation, degeneration or differentiation, in a subject, by administering to the subject an effective amount of a modulator (e.g., an agonist or antagonist) of a Delta3 activity. In one embodiment, an agonist or antagonist can modulate Delta3 protein levels, by, e.g., modulating expression of a Delta3 gene. A modulator can, for example, be a protein of the invention, or, alternatively, a nucleic acid of the invention. In other embodiments, the modulator is a peptide, antibody, peptidomimetic, or other small organic molecule.

For example, administration of a therapeutic comprised of a Delta3 agonist can be useful for promoting the tissue regeneration or repair needed to effectively treat a nerve injury, neurodegenerative disease, or neurodevelopmental disorder including but not limited to peripheral neuropathies, e.g., ACCPN, stroke, dementia, Alzheimer's disease, Parkinson's disease, Huntington's chorea, amylotrophic lateral sclerosis, and the like, as well as spinocerebellar degenerations.

Alternatively, administration of a Delta3 antagonist may be to effectively treat a neoplastic or hyperplastic disease, particularly of endothelial tissue.

Additionally, Delta3 agonists or antagonists may be used to treat various hematologic abnormalities such as neutropenia seen in patients undergoing chemotherapy, or immunodeficiency disorders such as AIDS. Delta3 nucleic acids, polypeptides or modulators thereof can also be utilized in treating or ameliorating a symptom of obesity and/or disorders that accompany or are exacerbated by an obese state, such as cardiovascular and circulatory disorders, metabolic abnormalities typical of obesity, such as hyperinsulinemia, insulin resistance, diabetes, including non-insulin dependent diabetes mellitus (NIDDM), insulin dependent diabetes mellitus (IDDM), and maturity onset diabetes of the young (MODY), disorders of energy homeostasis, disorders associated with lipid metabolism, such as cachexia, disorders associated with abnormal vasculogenesis (e.g., cancers, including, but not limited to, cancers of the epithelia (e.g., carcinomas of the pancreas, stomach, liver, secretory glands (e.g., adenocarcinoma), bladder, lung, breast, skin (e.g., fibromatosis or malignant melanoma), reproductive tract including prostate gland, ovary, cervix and uterus); cancers of the hematopoietic and immune system (e.g., leukemias and lymphomas); cancers of the central nervous, brain system and eye (e.g., gliomas, neuroblastoma and retinoblastoma); and cancers of connective tissues, bone, muscles and vasculature (e.g., hemangiomas and sarcomas)), disorders related to fetal development, in particular, disorders involving development of lung and kidney, lung-related disorders, atherosclerosis, e.g., the initiation and progression of atherosclerosis; and inflammatory-related disorders, e.g., asthma, allergy, and autoimmune disorders, neurological disorders, including developmental, cognitive and personality-related disorders, renal disorders, adrenal gland-related disorders; and disorders relating to skeletal muscle, such as dystrophic disorders.

The invention also provides methods for treating diseases or conditions associated with one or more specific Delta alleles, e.g., mutant allele, comprising administering to the subject an effective amount of a therapeutic compound. In one embodiment, the therapeutic compound is capable of compensating for the effect of the specific Delta allele. In another embodiment, the therapeutic compound is capable of modulating a Delta3 activity. In a one embodiment, the Delta allele is a Delta3 allele. For example, in one embodiment, the disease or condition is a neurological disease, e.g., ACCPN.

A further aspect of the present invention provides a method for determining whether a subject is at risk for developing a disease or condition, which is caused by or contributed to by an aberrant Delta3 activity, e.g. aberrant cell proliferation, degeneration or differentiation. In one embodiment, the disease or condition is a neurological disease, e.g., ACCPN. The method includes detecting, in a tissue of the subject, the presence or absence of a genetic lesion characterized by at least one of (i) a mutation of a gene encoding a Delta3 protein, e.g., as shown in SEQ ID Nos: 1, 3, 24, 26, 27, 29, 31, 33, 35, 37, 39, 41, 43 or 45, or the nucleotide sequence of the cDNA of a clone deposited with the ATCC® as Accession Number 98348, or a homolog thereof; or (ii) the mis-expression of a Delta3 gene. In one embodiments, detecting the genetic lesion includes ascertaining the existence of at least one of the following: a deletion of one or more nucleotides from a Delta3 gene; an addition of one or more nucleotides to the gene, a substitution of one or more nucleotides of the gene, a gross chromosomal rearrangement of the gene; an alteration in the level of a messenger RNA transcript of the gene; the presence of a non-wild type splicing pattern of a messenger RNA transcript of the gene; and/or a non-wild type level of the protein.

In a preferred embodiment, the invention provides a method for determining whether a subject has or is at risk of developing a disease or condition associated with a specific Delta3 allele, comprising determining the identity of a Delta3 allele in the subject. In an even more preferred embodiment, the disease or condition is a neurological disease, e.g., ACCPN. In another preferred embodiment, the disease is a vascular disorder. In another preferred embodiment, the disease is a neoplastic disorder. In another preferred embodiment the disease is a hematologic disorder. In another preferred embodiment the disease is an immunodeficiency disorder.

For example, detecting the genetic lesion or determining the identity of a Delta allele, e.g., a Delta3 allele, can include: (i) providing a probe/primer comprised of an oligonucleotide which hybridizes to a sense or antisense sequence of a Delta3 gene or naturally-occurring mutants thereof, or 5′ or 3′ flanking sequences naturally associated with the Delta3 gene; (ii) contacting the probe/primer with an appropriate nucleic acid containing sample; and (iii) detecting, by hybridization of the probe/primer to the nucleic acid, the presence or absence of the genetic lesion, e.g., by utilizing the probe/primer to determine the nucleotide sequence of the Delta3 gene and, optionally, of the flanking nucleic acid sequences. For instance, the primer can be employed in a polymerase chain reaction (PCR) or in a ligation chain reaction (LCR).

In another diagnostic method of the invention, at least a portion of a Delta3 gene of a subject is sequenced and the nucleotide sequence is compared to that of a wild-type Delta3 gene, to determine the presence of a genetic lesion. Another preferred diagnostic method of the invention is single strand conformation polymorphism (SSCP) which detects differences in electrophoretic mobility between mutant and wild-type nucleic acids.

In alternate embodiments, the diagnostic methods comprise determining the level of a Delta3 protein in an immunoassay using an antibody which is specifically immunoreactive with a wildtype or mutant Delta3 protein.

In another aspect, the present invention provides methods for detecting the presence of the activity or expression of a polypeptide of the invention in a biological sample by contacting the biological sample with an agent capable of detecting an indicator of activity such that the presence of activity is detected in the biological sample.

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of a polypeptide of the invention wherein a wild-type form of the gene encodes a polypeptide having the activity of the polypeptide of the invention.

Other features and advantages of the invention will be apparent from the following detailed description and claims.

### 3. DEFINITIONS

For convenience, the meaning of certain terms and phrases employed in the specification, examples, and appended claims are provided below.

The term “activity,” for the purposes herein refers to an activity exerted by a polypeptide of the invention on a responsive cell as determined, in vivo or in vitro, according to standard techniques. An activity can refer to an effector or antigenic function that is directly or indirectly performed by a Delta3 polypeptide (whether in its native or denatured conformation), or by any subsequence thereof. Effector functions include, for example, receptor binding or activation, induction of differentiation, mitogenic or growth promoting activity, induction of apoptosis, signal transduction, immune modulation, DNA regulatory functions and the like, whether presently known or inherent. Antigenic functions include possession of an epitope or antigenic site that is capable of binding antibodies raised against a naturally-occurring or denatured Delta3 polypeptide or fragment thereof. Accordingly, an activity of a Delta3 protein can be binding to a receptor, such as Notch. An activity of a Delta3 protein can also be modulation of cell proliferation and/or differentiation, or cell death in a target cell having an appropriate receptor. A target cell can be, e.g., a neural cell, an endothelial cell, or a cancer cell.

The term “aberrant Delta3 activity” or “abnormal Delta3 activity” is intended to encompass an activity of Delta3 which differs from the same Delta3 expression or activity in a healthy subject. An aberrant Delta3 activity can result, e.g., from a mutation in the protein, which results, e.g., in lower or higher binding affinity to a receptor. An aberrant Delta3 activity can also result from a lower or higher level of Delta3 on cells, which can result, e.g., from aberrant transcription, splicing, or translation of the Delta3 gene. For example, an aberrant Delta3 activity can result from an abnormal promoter activity. An aberrant Delta3 activity can also result from an aberrant signalling through the cytoplasmic domain of the Delta3 protein, such that, e.g., an aberrant signal is transduced. Aberrant signalling can result from a mutation in the cytoplasmic domain of Delta3 or, alternatively, from the contact with an abnormal cytoplasmic protein. An aberrant Delta3 activity can also result from contact of a Delta3 protein with an aberrant receptor, e.g., abnormal Notch protein.

The term “allele”, which is used interchangeably herein with “allelic variant” refers to alternative forms of a gene, nucleic acid or portions thereof, as well as to a polypeptide encoded by said gene, nucleic acid, or portion thereof. Nucleic acid alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

The term “allelic variant of a polymorphic region of a Delta3 gene” refers to a region of a Delta gene having one of several nucleotide sequences found in that region of the gene in other individuals, as well as to polypeptides encoded by nucleic acid molecules comprising said sequences.

The term “agonist”, as used herein, is meant to refer to an agent that upregulates (e.g., potentiates or supplements) Delta3 expression, levels and/or activity. It is to be understood that a Delta3 agonist can include a compound which increases signaling from a Delta3 protein, e.g., a compound bound to Delta3, such as a stimulatory form of a toporythmic protein or a small molecule. A Delta3 agonist can also, for example, be a compound which modulates the expression or activity of a protein which is located upstream or downstream of Delta3 and or which interacts with Delta3.

“Antagonist” as used herein is meant to refer to an agent that downregulates (e.g., suppresses or inhibits) Delta3 expression, levels and/or activity. A Delta3 antagonist can, for example, be a compound which decreases signalling from a Delta3 protein, e.g., a compound binding to Delta3 such as an inhibitory form of a toporythmic protein, or a small molecule. A Delta3 antagonist can include compounds that inhibit the interaction between a Delta3 protein and another molecule, e.g., a toporythmic protein. A Delta3 antagonist can also be a compound which modulates the expression or activity of a protein which is located upstream or downstream of Delta3 and/or which interacts with Delta3.

The term “antibody” as used herein refers to immunoglobulin molecules and immunologically active portions of immunoglobulin molecules, i.e., molecules that contain an antigen biding site which specifically binds an antigen, such as a polypeptide of the invention, e.g., an epitope of a polypeptide of the invention. A molecule The term “antibody” as used herein refers to immunoglobulin molecules and immunologically active portions of immunoglobulin molecules, i.e., molecules that contain an antigen binding site which specifically binds an antigen, such as a polypeptide of the invention, e.g., an epitope of a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

“Cells,” “host cells” or “recombinant host cells” are terms used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

“Complementary” sequences as used herein refer to sequences which have sufficient complementarity to be able to hybridize, forming a stable duplex.

A “delivery complex” shall mean a targeting means (e.g., a molecule that results in higher affinity binding of a gene, protein, polypeptide or peptide to a target cell surface and/or increased cellular uptake by a target cell). Examples of targeting means include: sterols (e.g., cholesterol), lipids (e.g., a cationic lipid, virosome or liposome), viruses (e.g., adenovirus, adeno-associated virus, and retrovirus) or target cell specific binding agents (e.g., ligands recognized by target cell specific receptors). Preferred complexes are sufficiently stable in vivo to prevent significant uncoupling prior to internalization by the target cell. However, the complex is cleavable under appropriate conditions within the cell so that the gene, protein, polypeptide or peptide is released in a functional form.

As is well known, genes for a particular polypeptide may exist in single or multiple copies within the genome of an individual. Such duplicate genes may be identical or may have certain modifications, including nucleotide substitutions, additions or deletions, which all still code for polypeptides having substantially the same activity. The term “DNA sequence encoding a Delta3 polypeptide” may thus refer to one or more genes within a particular individual. Moreover, certain differences in nucleotide sequences may exist between individual organisms, which are called alleles. Such allelic differences may or may not result in differences in amino acid sequence of the encoded polypeptide yet still encode a protein with the same biological activity.

The term “Delta3 therapeutic” refers to various compositions of Delta3 modulators (e.g., agonists or antagonists), such as polypeptides, antibodies, peptidomimetics, small molecules and nucleic acids which are capable of mimicking or potentiating (agonizing) or inhibiting suppressing (antagonizing) Delta3 expression, levels, or activity, e.g., which are capable of agonizing or antagonizing the effects of a naturally-occurring Delta3 protein.

The terms “Delta3 polypeptide” and “Delta3 protein” are intended to encompass, e.g., Delta3 polypeptides which have at least one activity of a native Delta3 polypeptide, or can, e.g., antagonize or agonize at least one biological activity of a native Delta3 polypeptide.

A “fusion protein” is a fusion of a first amino acid sequence encoding one of the subject Delta3 polypeptides with a second, heterologous amino acid sequence. In general, a fusion protein can be represented by the general formula X-Delta3-Y, wherein Delta3 represents a portion of the protein which is derived from one of the Delta3 proteins of the invention, and X and Y are independently absent or represent amino acid sequences which are heterologous to (that is, not related to) one of the Delta3 sequences in an organism, including naturally-occurring mutants. Among the Delta3 fusion protein is a Delta3-Ig fusion protein.

As used herein, the term “gene” or “recombinant gene”, as applied to Delta3, refers to a nucleic acid molecule comprising an open reading frame encoding one of the Delta3 polypeptides of the present invention. In one embodiment, these terms relate to a cDNa sequence including, but not limited to a nucelci acid sequence obtained via reverse transcription of an mRNA molecule.

The term “growth state” of a cell refers to the proliferative state of a cell as well as to its differentiative state. Accordingly, the term refers to the phase of the cell cycle in which the cell is, e.g., G0, G1, G2, prophase, metaphase, or telophase, as well as to its state of differentiation, e.g., undiffereniated, partially differentiated, or fully differentiated. Without wanting to be limited, differentiation of a cell is usually accompanied by a decrease in the proliferative rate of a cell.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are identical at that position. A degree of homology or similarity or identity between nucleic acid sequences is a function of the number of identical or matching nucleotides at positions shared by the nucleic acid sequences. A degree of identity of amino acid sequences is a function of the number of identical amino acids at positions shared by the amino acid sequences. Likewise, a degree of identity of nucleic acid sequences is a function of the number of identical nucleic acids at positions shared by the nucleic acid sequences.

Furthermore, a degree of homology or similarity of amino acid sequences is a function of the number of conserved amino acids at positions shared by the amino acid sequences. A sequence which is “unrelated” or “non-homologous” with one of the hDelta3 sequences of the present invention typically is a sequence which shares less than 40% identity, though preferably less than 25% identity with one of the hDelta3 sequences of the present invention.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). In one embodiment the two sequences are the same length. In another embodiment, the mouse Delta3 polypeptide is one amino acid longer than human Delta3.

Preferably, the determination of percent identity between two sequences is accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

As used herein, the term “hybridizes under stringent conditions” is intended to describe conditions for hybridization and washing under which nucleotide sequences at least 60% (65%, 70%, preferably 75% or more) identical to each other typically remain hybridized to each other. Such stringent conditions are known to those skilled in the art and can be found in Current Protocols in Molecular Biology, John Wiley & Sons, N.Y. (1989), 6.3.1-6.3.6, which describes aqueous and non-aqueous methods, either of which can be used. Another preferred, non-limiting example of stringent hybridization conditions are hybridization in 6× sodium chloride/sodium citrate (SSC) at about 45_C., followed by one or more washes in 2.0× SSC at 50° C. (low stringency) or 0.2× SSC, 0.1% SDS at 50-65_C. (high stringency). Another preferred example of stringent hybridization conditions are hybridization in 6× sodium chloride/sodiumcitrate (SSC) at about 45□C., followed by one or more washes in 0.2× SSC, 0.1% SDS at 50□C. Another example of stringent hybridization conditions are hybridization in 6× sodium chloride/sodium citrate (SSC) at about 45□C., followed by one or more washes in 0.2× SSC, 0.1% SDS at 55□C. A further example of stringent hybridization conditions are hybridizationin 6× sodium chloride/sodium citrate (SSC) at about 45□C., followed by one or more washes in 0.2× SSC, 0.1% SDS at 60□C. Preferably, stringent hybridization conditions are hybridization in 6× sodium chloride/sodium citrate (SSC) at about 45□C., followed by one or more washes in 0.2× SSC, 0.1% SDS at 65□C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65□C., followed by one or more washes at 0.2× SSC, 1% SDS at 65□C. In one embodiment, an isolated nucleic acid molecule of the invention that hybridizes under stringent conditions to the sequence of SEQ ID NOs: 1, 3, 24, 26, 27, 29, 31, 33, 35, 37, 39, 41, 43 or 45, or complement thereof, corresponds to a naturally-occurring nucleic acid molecule.

The term “interact” as used herein is meant to include detectable interactions between molecules, such as can be detected using, for example, a yeast two hybrid assay. The term interact is also meant to include “binding” interactions between molecules. Interactions may be, e.g., protein-protein, protein-nucleic acid, protein-small molecule, or nucleic acid-small molecule in nature.

The term “modulation” as used herein refers to both upregulation, i.e., stimulation, and downregulation, e.g., suppression, of a response.

The term “mutated gene” refers to an allelic form of a gene, which is capable of altering the phenotype of a subject having the mutated gene relative to a subject which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous (for that gene) subject, the mutation is said to be co-dominant.

As used herein, a “naturally-occurring” nucleic acid molecule refers to an RNA or DNA molecule having a nucleotide sequence that occurs in nature (e.g., encodes a natural protein).

The “non-human animals” of the invention include mammalians such as rodents, non-human primates, sheep, dog, cow, chickens, amphibians, reptiles, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant is expressed in some but not all cells of the animal. The term “tissue-specific chimeric animal” indicates that one of the recombinant Delta3 genes is present and/or expressed or disrupted in some tissues but not others.

As used herein, the term “nucleic acid molecule” is intended to include DNA molecules and RNA molecules (e.g., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. In other embodiments, the “isolated” nucleic acid is free of intron sequences. For example, in various embodiments, the isolated nucleic acid molecule preferably includes no more than 10 kilobases (kb), and more preferably, contains less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, viral material, or culture medium when produced by recombinant techniques, or substantially free of chemical precursors or other chemicals when chemically synthesized.

The terms “protein”, “polypeptide” and “peptide” are used interchangably herein. The term “substantially free of other cellular proteins” (also referred to herein as “contaminating proteins”) or “substantially pure or purified preparations” are defined as encompassing preparations of Delta3 polypeptides having less than about 20% (by dry weight) contaminating protein, and preferably having less than about 5% contaminating protein. Functional forms of the subject polypeptides can be prepared, for the first time, as purified or isolated preparations by using a cloned gene as described herein. By “purified” or “isolated,” it is meant, when referring to a protein of the invention, that the indicated molecule is present in the substantial absence of other biological macromolecules, such as other proteins. The term “purified” or “isolated” as used herein preferably means at least 80% by dry weight, more preferably in the range of 95-99% by weight, and most preferably at least 99.8% by weight, of biological macromolecules of the same type present (but water, buffers, and other small molecules, especially molecules having a molecular weight of less than about 5000, can be present). The term “pure” or “isolated” as used herein preferably has the same numerical limits as “purified” or “isolated” immediately above. “Isolated” and “purified” do not encompass either natural materials in their native state or natural materials that have been separated into components (e.g., in an acrylamide gel) but not obtained either as pure (e.g., lacking contaminating proteins, or chromatography reagents such as denaturing agents and polymers, e.g., acrylamide or agarose) substances or solutions. In preferred embodiments, purified or isolated Delta3 preparations will lack any contaminating proteins from the same animal from which Delta3 is normally produced, as can be accomplished by recombinant expression of, for example, a human Delta3 protein in a non-human cell.

As used herein, the term “tissue-specific promoter” means a DNA sequence that serves as a promoter, i.e., regulates expression of a selected DNA sequence operably linked to the promoter, and which effects expression of the selected DNA sequence in specific cells of a tissue, such as cells of cardiac, hepatic or pancreatic origin, neuronal cells, or immune cells. The term also covers so-called “leaky” promoters, which regulate expression of a selected DNA primarily in one tissue, but cause expression in other tissues as well.

“Transcriptional regulatory sequence” is a generic term used throughout the specification to refer to DNA sequences, such as initiation signals, enhancers, and promoters, which induce or control transcription of protein coding sequences with which they are operably linked. In certain embodiments, transcription of one of the recombinant Delta3 genes is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring forms of Delta3 proteins.

As used herein, the term “transfection” means the introduction of a nucleic acid, e.g., an expression vector, into a recipient cell by nucleic acid-mediated gene transfer. “Transformation”, as used herein, refers to a process in which a cell's genotype is changed as a result of the cellular uptake of exogenous DNA or RNA, and, for example, the transformed cell expresses a recombinant form of a Delta3 polypeptide or, in the case of anti-sense expression from the transferred gene, the expression of a naturally-occurring form of the Delta3 protein is disrupted.

As used herein, the term “transgene” means a nucleic acid sequence (encoding, e.g., one of the Delta3 polypeptides, or an antisense transcript thereto), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can include one or more transcriptional regulatory sequences and any other nucleic acid, such as introns, that may be necessary for optimal expression of a selected nucleic acid.

A “transgenic animal” refers to any non-human animal, preferably a non-human mammal, bird or an amphibian, in which one or more of the cells of the animal contain heterologous nucleic acid (“transgene”) introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of the Delta3 proteins, e.g., either agonistic or antagonistic forms. However, transgenic animals in which the recombinant Delta3 gene is silent are also contemplated, as for example, the FLP or CRE recombinase dependent constructs described below. Moreover, “transgenic animal” also includes those recombinant animals in which gene disruption of one or more Delta3 genes is caused by human intervention, including both recombination and antisense techniques. A “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. However, the invention is intended to include such other forms of expression vectors which serve equivalent functions and which become known in the art subsequently hereto.

The term “treating” as used herein is intended to encompass curing as well as ameliorating at least one symptom of the condition or disease.

The percent identity between two sequences can be determined using techniques similar to those described above, with or without allowing gaps. In calculating percent identity, typically only exact matches are counted.

### 3. BRIEF DESCRIPTION OF THE DRAWINGS

FIG. 1 shows a DNA sequence of the human Delta3 nucleotide sequence including 5′ and 3′ noncoding sequences (SEQ ID NO: 1), as well as the deduced amino acid sequence of the human Delta3 protein (SEQ ID NO: 2). The various domains of the protein are indicated, i.e., DSL domain=“DSL”; EGF repeats 1-8 (that is, EGF-like domains)=“I” through “VIII,” respectively; and the transmembrane domain=“TM”.

FIG. 2 shows a multiple sequence alignment of the novel human Delta3 protein (h-Delta3 ) (SEQ ID NO: 2) with the mouse Delta1 protein (m-delta1) (SEQ ID NO: 4), the rat Delta1 protein (r-delta1) (SEQ ID NO: 5), the partial human Delta1 (WO 97/01571) protein (SEQ ID NO: 6), the Xenopus Delta1 protein (x-delta1) (SEQ ID NO: 7), the chick Delta1 protein (c-delta1) (SEQ ID NO: 8), the zebrafish Delta1 protein (z-delta1) (SEQ ID NO: 9) the Xenopus Delta2 protein (x-delta2) (SEQ ID NO: 10) and the Drosophila Delta1 protein (d-delta1) (SEQ ID NO: 11). Conservation of the Delta3 Serrated lag-2 (DSL) domain, the epidermal growth factor-like (EGF) repeats and the transmembrane domain (TM) is indicated. The GenBank Accession NO: of each of these Delta proteins (with the exception of the partial human sequence, which is not in GenBank) is indicated in Table I, below.

FIG. 3 shows a phylogenic tree indicating the relationship of hDelta3 with the partial human Delta1 (WO 97/01571, Jan. 16, 1997) protein, the mouse Delta1 protein (m-delta1), the rat Delta1 protein (r-delta1), the Xenopus Delta1 protein (x-delta1), the chick Delta1 protein (c-delta1), the Xenopus Delta2 protein (x-delta2), the zebrafish Delta1 protein (z-delta1), and the Drosophila Delta1 protein (d-delta1). The GenBank Accession NO: of each of these Delta proteins (with the exception of the partial human sequence, which is not in GenBank) is indicated in Table I.

FIG. 4 shows a DNA sequence of the murine Delta3 nucleotide sequence including 5′ and 3′ non-coding sequences (SEQ ID NO: 24); and the deduced amino acid sequence of the murine Delta3 protein (SEQ ID NO: 25). The various domains of the protein are indicated, i.e., DSC domain=“DSC”; EGF repeats 1-8 (that is, EGF-like domains) 1-8=“I” through “VIII,” respectively; and the transmembrane domain=“TM”.

FIG. 5 shows an alignment of the amino acid sequences of human Delta3 (SEQ ID 15 NO: 2) and mouse Delta3 (SEQ ID NO: 25). The alignment was performed with BLAST, Blosum62 using a gap weight of 12, and length weight of 4.

### 4. DETAILED DESCRIPTION OF THE INVENTION

4.1 Human and Mouse Delta3

The present invention is based at least in part on the discovery of a novel gene encoding a human Delta protein referred to herein as “hDelta3 ” polypeptide, and the mouse equivalent referred to herein as “mDelta3”. An exemplary hDelta3 has been deposited with the ATCC® on Mar. 5, 1997 and has been assigned ATCC® GenBank Accession Number 98348. The human Delta3 gene maps to human chromosome 15.

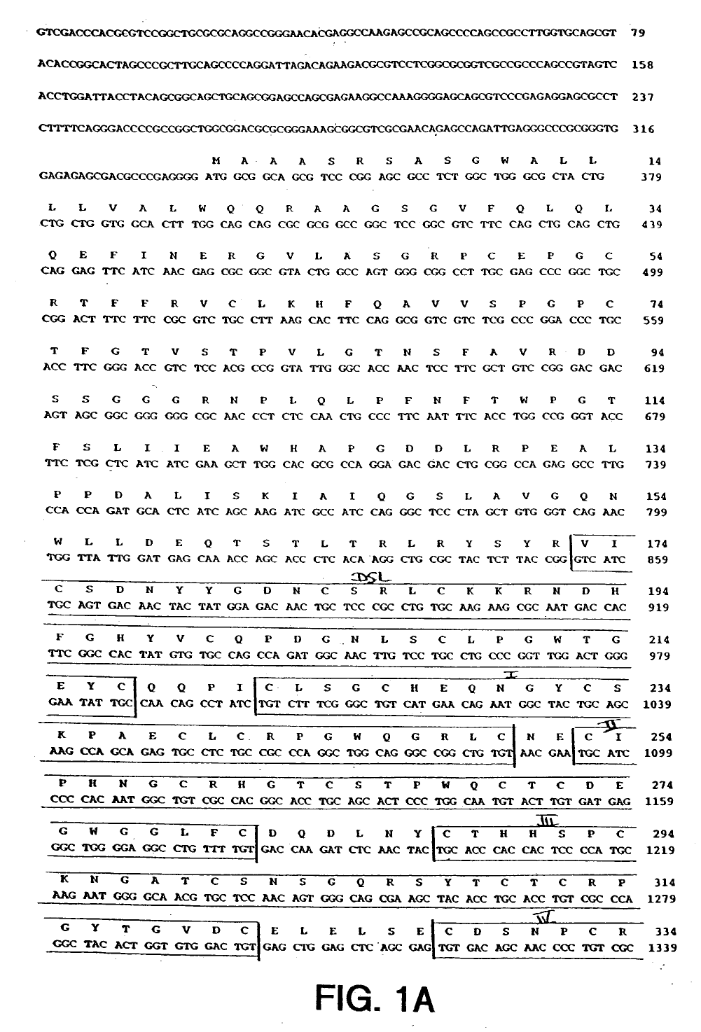

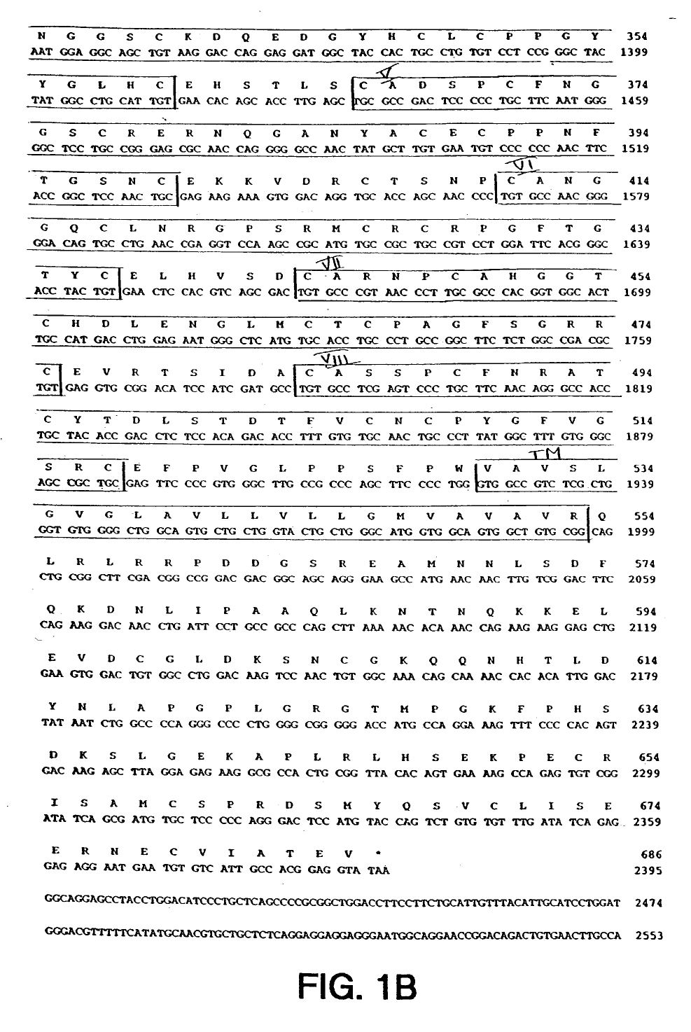

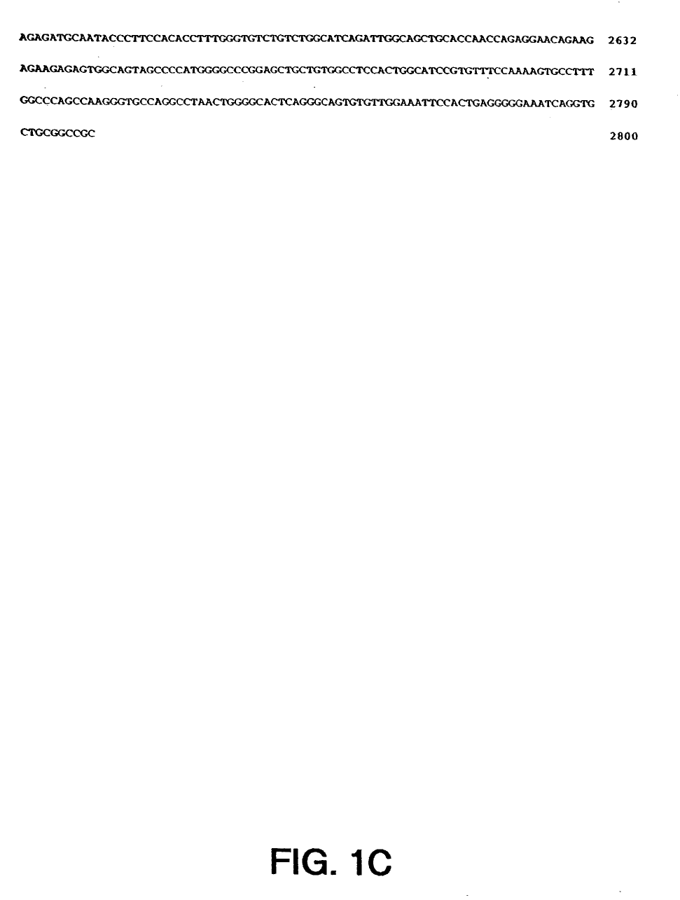

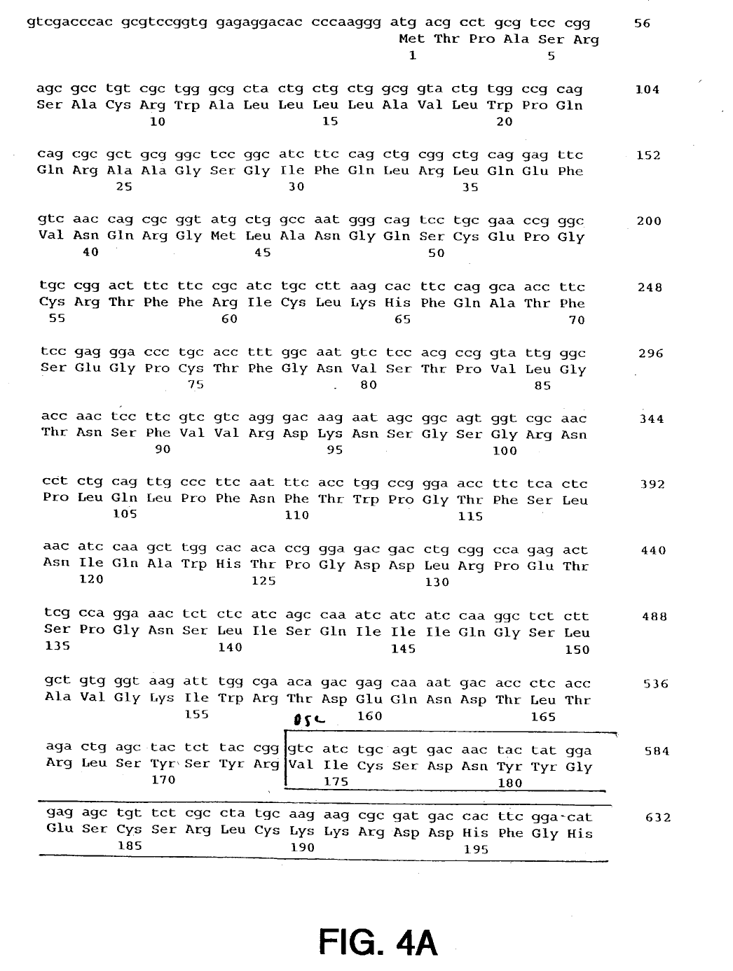

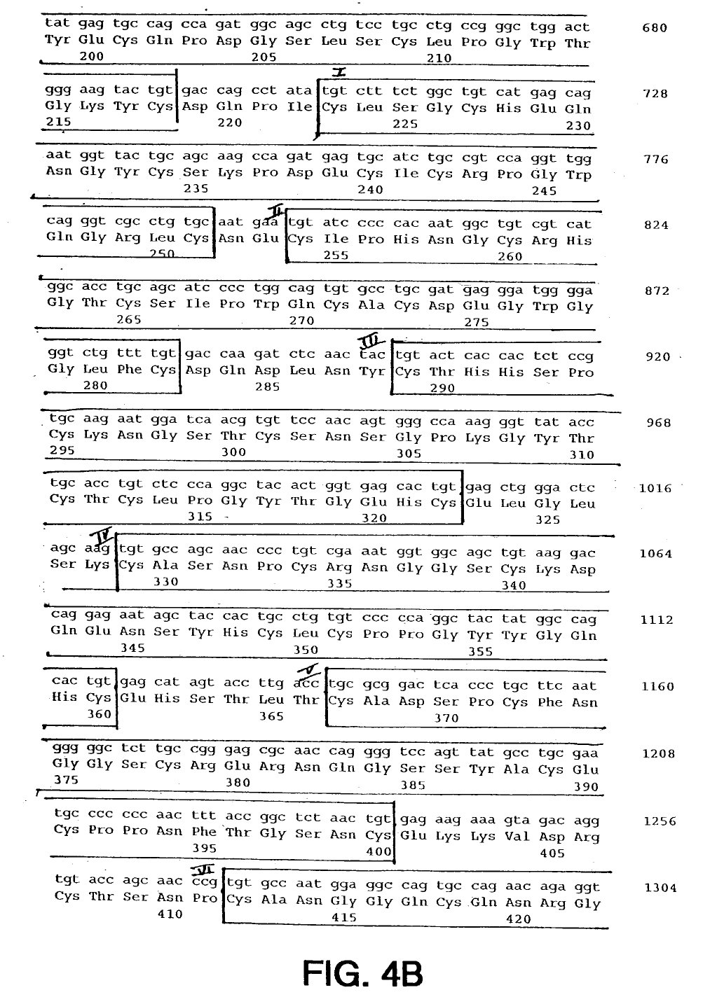

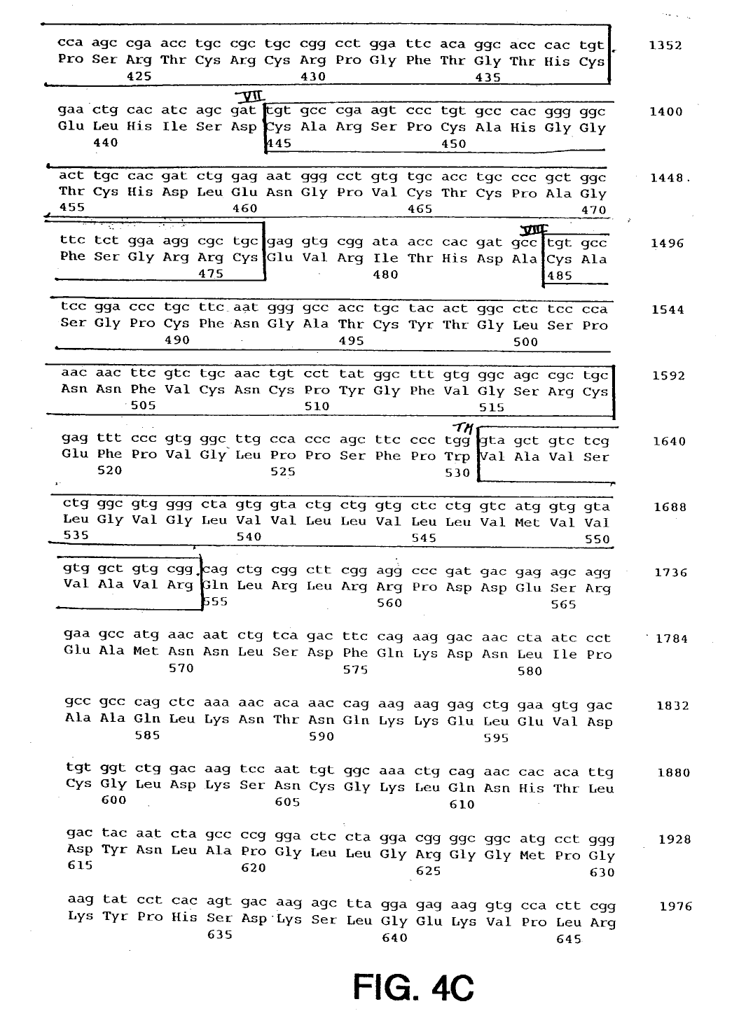

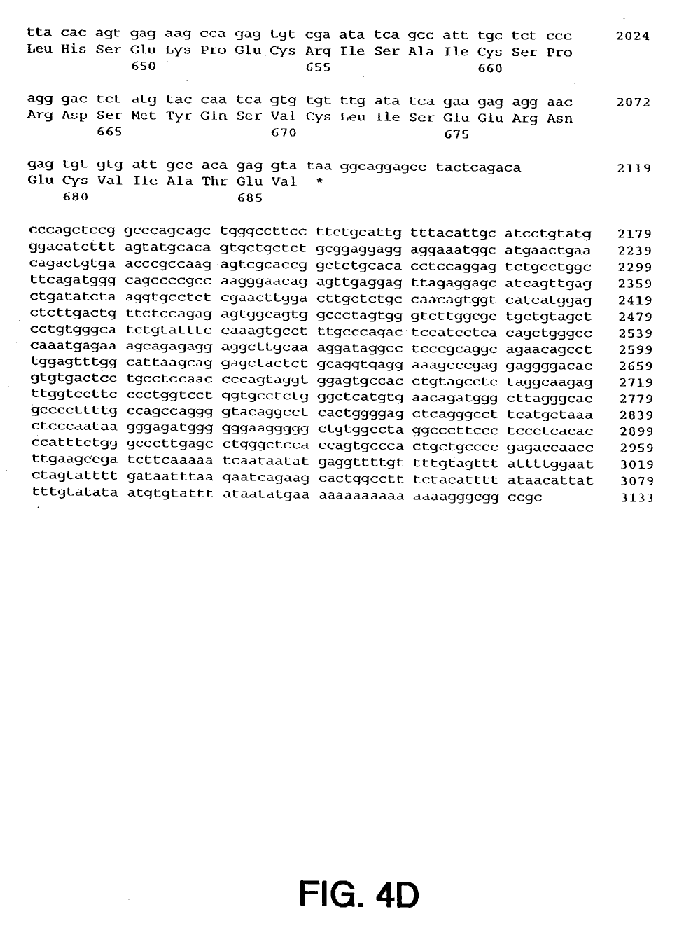

FIG. 1 shows the DNA sequence of human Delta3 including 5′ and 3′ non-coding sequences (SEQ ID NO: 1), the coding sequence (SEQ ID NO: 3), as well as the deduced amino acid sequence of the human Delta3 protein (SEQ ID NO: 2). FIG. 4 shows the DNA sequence of mouse Delta3 including 5′ and 3′ non-coding sequences (SEQ ID NO: 24) and the coding sequence (SEQ ID NO: 26). FIG. 4 shows the deduced amino acid sequence of the mouse Delta3 protein (SEQ ID NO: 25).

Human Delta3 is expressed in endothelial cells and in fact was cloned from a human microvascular endothelial cell library. Northern blot analysis of RNA prepared from a number of different human tissues, indicate that a 3.5 kb Delta3 mRNA transcript is present in fetal brain, lung, liver and kidney, and adult heart, placenta, lung, skeletal muscle, kidney, pancreas, spleen, thymus, prostate, testis, ovary, small intestine and colon. Low levels of Delta3 mRNA were also detected in adult brain and adult liver. However, no Delta3 mRNA was detected in peripheral blood leukocytes. These results indicate that Delta3 is expressed in a tissue-specific manner. Further, expression in human microvascular endothelial cells was found to be up-regulated (about 2-3 fold) in cells that had been stimulated with certain growth factors (e.g., basic fibroblast growth factor (bFGF) or vascular endothelial growth factor (VEGF)). In addition, strong expression of human Delta3 was observed in the colorectal carcinoma cell line, SW480. Furthermore, expression of hDelta3 has been shown to be induced in response to proliferation and differentiation signals (See Examples). Thus, the Delta3 gene, in particular, the hDelta3 gene, is a gene whose expression in a cell changes with the state of proliferative and/or differentiative state of cells.

In situ hybridization was performed on a wide range of murine adult and embryonic tissues using a probe complementary to mRNA of mDelta3. Expression was most abundant and widespread during embryogenesis. Strongest expression was observed in the eye of all the embryonic ages tested. Signal in a pattern suggestive of neuronal expression was not observed in any other tissues making the expression in the eye unique. Ubiquitous expression was also detected in lung, thymus and brown fat during embryogenesis. A multifocal, scattered signal was also observed throughout the embryo. This signal pattern was more focused in the cortical region of the kidney and outlining the intestinal tract. Adult expression was highest in the ovary and the cortical regions of the kidney and adrenal gland. This is consistent with Delta3's role as a regulator of cell growth and/or differentiation.

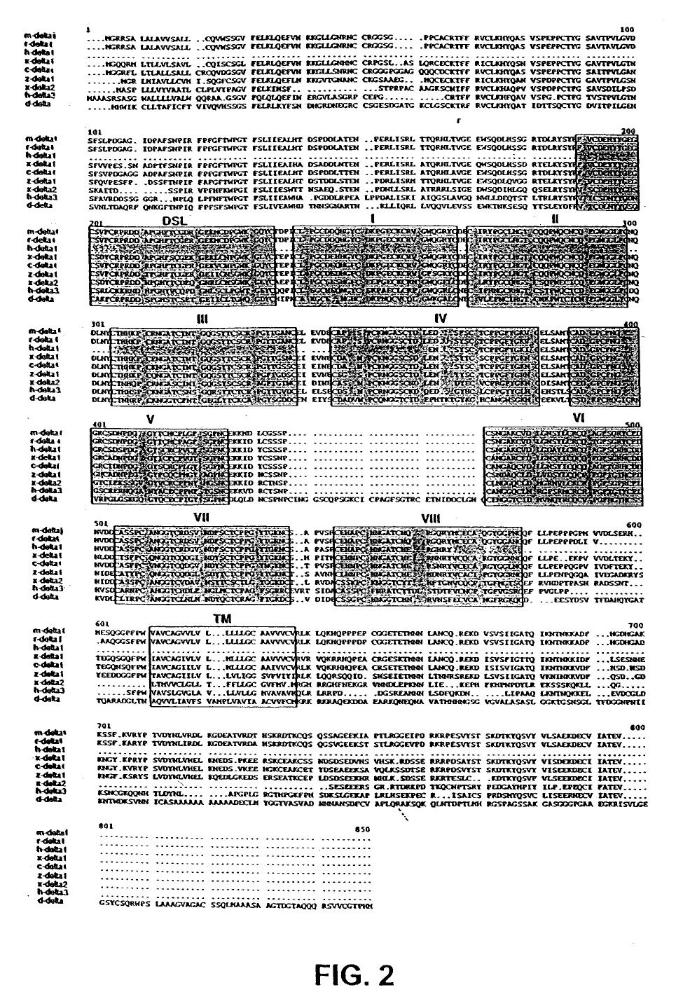

As predicted from the nucleotide sequence of the nucleic acid encoding hDelta3, the novel, full-length hDelta3 polypeptide comprises 685 amino acids and is similar and structure to Delta proteins obtained from other organisms (See FIG. 2 and discussed below). An amino acid sequence analysis of Delta3 proteins predicts that the protein comprises at least the structural domains described herein. First, human and mouse Delta3 have a signal peptide, corresponding to amino acid 1 to amino acid 16, amino acid 1 to amino acid 17, amino acid 1 to amino acid 18, amino acid 1 to amino acid 19 or amino acid 1 to amino acid 20 of SEQ ID NOs: 2 or 25. The signal sequence is normally cleaved during processing of the mature protein. In such embodiments of the invention, the domains and the mature protein resulting from cleavage of such signal peptides are also included herein. For example, the cleavage of a signal sequence consisting of amino acids 1 to 17 results in an extracellular domain consisting of amino acids 18 to 529 of SEQ ID No. 2 and the mature Delta3 polypeptide corresponding to amino acids 18 to 685 of SEQ ID NO: 2.

Second, human and mouse Delta3 have protein interaction domains such as a Delta Serrated lag-2 (DSL) motif corresponding to amino acid 173 to amino acid 217 of SEQ ID NO: 2 (FIG. 1) and amino acid 174 to amino acid 218 of SEQ ID NO: 25 (FIG. 4), as well as eight epidermal growth factor (EGF)-like repeats corresponding essentially to the sequences indicated in FIG. 2.

In addition, Delta3 proteins have a transmembrane domain, i.e., in human Delta3 the transmembrane domain corresponds to about amino acid 530 to about amino acid 553 of SEQ ID NO: 2 (FIG. 1), and in mouse Delta3, amino acid 531 to amino acid 554 of SEQ ID NO: 25. Delta3 proteins have also have a cytoplasmic domain corresponding to about amino acid 554 to about amino acid 685 of SEQ ID NO: 2 (FIG. 1) or amino acid 555 to amino acid 686 of SEQ ID NO: 25. Accordingly, sequence analysis for conserved domains of Delta3 amino acid sequence shows that the protein is likely a transmembrane protein having an extracellular domain corresponding to about amino acid 1 to about amino acid 529 of SEQ ID NO: 2 (FIG. 1), about amino acid 18 to about amino acid 529 of SEQ ID NO: 2 (FIG. 1), amino acid 1 to about amino acid 530 of SEQ ID NO: 25, or amino acid 18 to 530 of SEQ ID NO: 25, said extracellular domain comprising a DSL motif and eight EGF-like domains. The Delta3 protein further comprises a transmembrane domain and a cytoplasmic domain.

Human Delta3 protein is similar in structure and in sequence to the Delta proteins identified in Drosophila, Xenopus, zebrafish, chicken, rat, mouse, rat, and human. An alignment of the amino acid sequences of other known Delta proteins is shown in FIG. 2. This alignment contains the following Delta proteins: a mouse Delta1 protein (m-delta1), rat Delta-1 protein (r-delta1), a human Delta-1 protein (h-delta1), a Xenopus Delta1 protein (x-delta1), a chicken Delta1 protein (c-delta1), a zebrafish Delta1 protein (z-delta1), a second Xenopus Delta protein (x-delta2), as well as the human Delta3 protein (h-delta3), and a Drosophila Delta1 protein (d-delta). The amino acid sequence of h-delta1 is the amino acid sequence published in PCT Publication WO 97/01571 (Jan. 16, 1997) which is incomplete and contains numerous errors, as stated in the application. Since the amino acid sequence alignment has been done using the pileup computer program (GCG Package), the order of the amino acid sequences in the figure reflects the relative identity between the different Delta proteins. Accordingly, the Drosophila protein, which corresponds to the bottom sequence in the alignment is most distant from the other Delta proteins.

FIG. 2 shows that hDelta3, which is listed second to the last, is the second most distant Delta protein from the previously identified mouse, rat, human, Xenopus, zebrafish, and chicken delta protein. Accordingly, hDelta3 protein is significantly different from the previously described human Delta protein, as well as the Delta proteins from the other species. Interestingly, the hDelta3 protein has an amino acid sequence which is equally distant from both Xenopus proteins, i.e., Delta1 and Delta2, suggesting that hDelta3 does not correspond to either of the Xenopus Delta proteins. Therefore, the newly isolated polypeptide has been termed hDelta3 and the previously identified mouse, rat, human, zebrafish, and Xenopus Delta proteins are termed Delta1 proteins herein and the two Xenopus proteins are termed Delta1 and Delta2 proteins. The difference between hDelta3 protein and previously isolated Delta proteins can also be visualized by comparing the percentage similarity or identity between hDelta3 and the previously identified Delta1 and Delta2 proteins on one hand (Table I), and the percent similarity or identity of a Delta1 protein with the other Delta1 and Delta2 proteins (Table II).

A hallmark of Notch ligands such as Jagged-1, is the ability to block the differentiation of the C2C12 cell line from myoblasts into myotubes when co-cultured with NIH3T3 cells under low mitogenic conditions. When C2C12 cells were co-cultured with NIH3T3 cells, which were engineered to express hDelta3, differentiation of C2C12 cells from myoblasts to myotubes was blocked in a similar fashion as has been described for other Notch ligands such as Jagged-1. Therefore, the hDelta3 gene is likely to encode a polypeptide which functions as a bona fide Notch ligand. Indeed, the data presented in Section 5.6, below, indicates that hDelta3 is a bona fide Notch ligand.

Table I indicates the percent similarity and identity between human Delta3, the Delta1 disclosed in PCT Publication No. WO 97/01571 (Jan. 16, 1997) and non-human Delta1 proteins. Since the amino acid sequence of the human Delta1 protein that is disclosed in PCT Publication No. WO 97/01571 (Jan. 16, 1997) is incomplete, the percentage similarity and identity was determined using a portion of the human Delta1 amino acid sequence which seems most reliable. The portion of the amino acid sequence used corresponds to amino acids 214-370 of the human Delta1 amino acid sequence shown in FIG. 14 of the PCT Publication No. WO 97/01571 (Jan. 16, 1997).

TABLE I
Percentage similarity between the amino acid sequence of
human Delta3 (SEQ ID NO: 2) and that of the various Delta proteins

| | GenBank Accession NO: | | % identity | % similarity |
|---|---|---|---|---|
| human Delta1 | N.A. | (SEQ ID NO: 6) | 50 | 66 |
| mouse Delta1 | X80903 | (SEQ ID NO: 4) | 53 | 70 |
| rat Delta1 | U78889 | (SEQ ID NO: 5) | 54 | 70 |
| chicken Delta1 | U26590 | (SEQ ID NO: 8) | 52 | 68 |
| Xenopus Delta1 | L42229 | (SEQ ID NO: 7) | 51 | 68 |
| zebrafish Delta1 | Y11760 | (SEQ ID NO: 9) | 48 | 67 |
| Xenopus Delta2 | U70843 | (SEQ ID NO: 10) | 47 | 65 |
| Drosophila Delta1 | AA142228 | (SEQ ID NO: 11) | 40 | 58 |
| hDelta-like (dlk) | U15979 | | 33 | 55 |

Table II indicates the percent similarity and identity between human Delta1 disclosed in PCT Publication No. WO 97/01571 (1997) and non-human Delta1 proteins. Since the amino acid sequence of the human Delta1 protein that is disclosed in PCT Publication No. WO 97/01571 (1997) is incomplete, the percentage similarity and identity was determined using a portion of the human Delta1 amino acid sequence which seems most reliable. The portion of the amino acid sequence used corresponds to amino acids 214-370 of the human Delta1 amino acid sequence shown in FIG. 14 of the PCT application.

TABLE II
Percentage similarity between human Delta1
and the various non-human Delta1 or Delta2 proteins

| | GenBank Accession | | % identity | % similarity NO: |
|---|---|---|---|---|
| human Delta1 | N.A. | (SEQ ID NO: 6) | 100 | 100 |
| mouse Delta1 | X80903 | (SEQ ID NO: 4) | 86 | 95 |
| rat Delta1 | U78889 | (SEQ ID NO: 5) | 88 | 94 |
| chicken Delta1 | U26590 | (SEQ ID NO: 8) | 85 | 89 |
| Xenopus Delta1 | L42229 | (SEQ ID NO: 7) | 78 | 84 |
| zebrafish Delta1 | Y11760 | (SEQ ID NO: 9) | 69 | 80 |
| Xenopus Delta2 | U70843 | (SEQ ID NO: 10) | 57 | 70 |
| Drosophila Delta1 | AA142228 | (SEQ ID NO: 11) | 45 | 62 |
| hDelta-like (dlk) | U15979 | | 37 | 55 |

Accordingly, Table I indicates that hDelta3 is only approximately 66% similar to the human Delta1 protein; approximately 70% similar to the mouse Delta1 protein; approximately 70% similar to the rat Delta1 protein; approximately 68% similar to the chick Delta1 protein; approximately 68% similar to the Xenopus Delta1 protein, approximately 70% similar to the Xenopus Delta2 protein and approximately 58% similar to the Drosophila Delta1 protein. However, as shown in Table II, the human-Delta1 protein is very similar to the mouse, rat, chick, Xenopus, zebrafish, and Drosophila Delta1 and the Xenopus Delta2 proteins. In addition, mouse and rat Delta1 proteins are about 95% similar. Thus, the amino acid sequence of the orthologs of the Delta1 protein share greater similarity and identity with each other than with the human Delta3 protein of the invention, indicating that at least two families of Delta proteins exist.

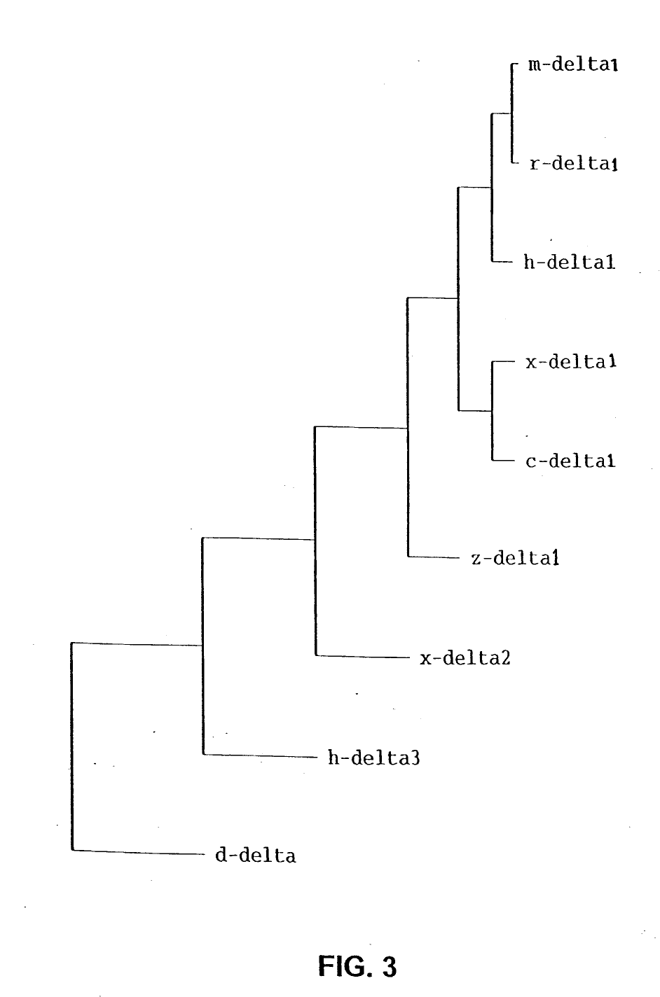

The difference between the newly isolated hDelta3 protein and the previously identified Delta1 and Delta2 proteins can also be seen by creating a phylogenic tree using the Growtree Phylogram computer program (GCG Package). The result of this analysis, which is shown in FIG. 3, indicate that h-Delta3 is on a different “branch” in the phylogenic tree from the other Delta proteins, thus confirming that hDelta3 protein is more distant from the other Delta1 and Delta2 proteins than they are distant from each other. According to the analysis, and as predicted by the sequence alignment, only the Drosophila Delta protein is more distantly related to the previously identified mouse, rat, Xenopus, chicken, zebrafish and human Delta proteins than hDelta3. Thus, the newly isolated hDelta3 protein is a member of a different subspecies of the family of Delta proteins.

Notwithstanding that each animal species is likely to have at least two or three members (e.g., Delta1, Delta 2, and Delta3), it can be seen from FIG. 2, that the DSL region, the eight EGF repeats and the TM appear to be highly conserved throughout. However, as can be seen in FIG. 2, these domains of the hDelta3 protein differ more from the corresponding domains in the other Delta proteins than the corresponding domains in the other Delta differ from one another.

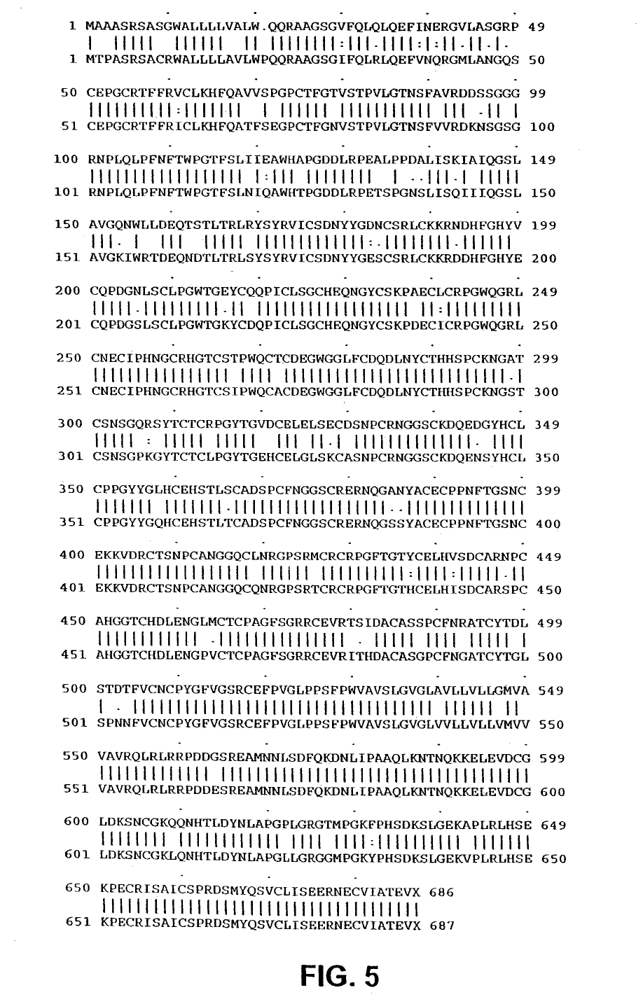

FIG. 5 shows a comparison of human and mouse Delta3, wherein the polypeptides are 86.6% identical and 88.2% similar. The polypeptides were aligned with the BLAST program using Blosum62, gap weight 12 and length weight 4. One gap was introduced at amino acid position twenty one due to an extra codon present in mouse Delta3. The skilled artisan will appreciate that the domains identified in FIGS. 2 and 3 with respect to the Delta polypeptides are also present in corresponding positions in mouse Delta3.

Furthermore, as set forth in the examples presented below, Delta3 has been localized to human chromosome 15 in a region close to the framework markers D15S1244 and D15S144. Interestingly, the region on chromosome 15 that is flanked by the markers D15S1040 and D15S118 has been shown to be genetically linked with the disease called Agenesis of the Corpus Callosum with Peripheral Neuropathy (ACCPN) (Casaubon et al. (1996) Am J. Hum. Genet. 58:28). No specific gene has so far been linked to this disease. Accordingly, since Delta3 is localized to a chromosomal region genetically linked to ACCPN and Delta3 is a member of the Notch signaling pathway, defects in which have been associated with a number of neurological diseases or conditions, Delta3 is likely to be the gene involved in ACCPN.

ACCPN, which is also termed Andermann syndrome (MIM 218000), is an autosomal recessive disorder that occurs with a high prevalence in the French Canadian population in the Charlevoix and Saguenay-Lac St Jean region in Quebec. The disease is characterized by a progressive peripheral neuropathy caused by axonal degeneration and a central nervous system (CNS) malformation characterized by the absence of hypoplasia of the corpus callosum. The disorder appears early in life, is progressive and results in death in the third decade of life of the subject.

Accordingly, certain aspects of the present invention relate to Delta3 proteins, nucleic acid molecules encoding Delta3 proteins, antibodies immunoreactive with Delta3 proteins, and preparations of such compositions. In addition, drug discovery assays are provided for identifying agents that modulate the biological function of Delta proteins, e.g., Delta3 proteins (i.e. agonists or antagonists), such as by binding to Delta3 or by altering the interaction of Delta3 with either downstream or upstream elements in the Delta/Notch signal transduction pathway by altering the interaction between Delta3 and a Delta3 binding protein. Such agents can be useful therapeutically, for example, to alter cell growth and/or differentiation or induction of apoptosis. Moreover, the present invention provides diagnostic and therapeutic assays and reagents for detecting and treating disorders involving an aberrant Delta3 activity, for example, aberrant expression (or loss thereof) of Delta3 gene or which are associated with a specific Delta allele, e.g., a Delta3 allele. Other aspects of the invention are described below or will be apparent to one of skill in the art in light of the present disclosure.

4.2. Isolated Nucleic Acid Molecules of the Present Invention

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules.

As described below, one aspect of the invention pertains to isolated nucleic acids comprising nucleotide sequences encoding Delta3 polypeptides, and/or equivalents of such polypeptides or nucleic acids. The “term equivalent” is understood to include nucleotide sequences encoding functionally equivalent Delta3 polypeptides or functionally equivalent peptides having an activity of a Delta3 protein such as described herein. Equivalent nucleotide sequences also include sequences that differ by one or more nucleotide substitutions, additions or deletions, such as allelic variants, and will, therefore, include, for example, sequences that differ from the nucleotide sequence of the Delta3 nucleic acid sequence shown in any of SEQ ID NOs: 1, 3, 24, 26, 27, 29, 31, 33, 35, 37, 39, 41, 43 or 45 due to the degeneracy of the genetic code.

A nucleic acid molecule of the present invention, e.g., a nucleic acid molecule having the nucleotide sequence of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, or a complement thereof, can be isolated using standard molecular biology techniques and the sequence information provided herein. Using all or a portion of the nucleic acid sequences of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, or a portion thereof. A nucleic acid molecule which is complementary to a given nucleotide sequence is one which is sufficiently complementary to the given nucleotide sequence that it can hybridize to the given nucleotide sequence under the conditions set forth herein, thereby forming a stable duplex.

Moreover, a nucleic acid molecule of the invention can comprise only a portion of a nucleic acid sequence encoding a full-length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologs in other cell types, e.g., from other tissues, as well as homologs from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, or of a naturally-occurring mutant of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a biologically active portion of a polypeptide of the invention can be prepared by isolating a portion of any of SEQ ID NOs: 2, 25, 28, 30, 32, 34, 36, 38, 40, 42, 44 or 46, or the amino acid sequence encoded by the cDNA of a clone deposited with the ATCC® as Accession Number 98348 expressing the encoded portion of the polypeptide protein (e.g., by recombinant expression in vitro) and assessing the activity of the encoded portion of the polypeptide.

The invention further encompasses nucleic acid molecules that differ from the nucleotide sequence of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, due to degeneracy of the genetic code and thus encode the same protein as that encoded by the nucleotide sequence of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348.

In addition to the nucleotide sequences of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, it will be appreciated by those skilled in the art that DNA sequence polymorphisms that lead to changes in the amino acid sequence may exist within a population (e.g., the human population). Such genetic polymorphisms may exist among individuals within a population due to natural allelic variation. Such allelic variants of a polymorphic region of a Delta3 gene are also included as part of the present invention. For example, the human gene for Delta3 was mapped to chromosome 15, between markers D15S1244 and D15S144, and therefore, human Delta3 family members can include nucleotide sequence polymorphisms (e.g., nucleotide sequences that vary from SEQ ID NO: 1, 3, 24, 26, 27, 29, 31, 33, 35, or 37) that map to this chromosome 15 region (i.e., between framework regions D15S1244 and D15S144), as well as polypeptides encoded therefrom.

Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologs of various species may be non-essential for activity and thus would be likely targets for alteration. Alternatively, amino acid residues that are conserved among the orthologs of various species (e.g., murine and human) may be essential for activity and thus would not be likely targets for alteration.

In one embodiment of a nucleotide sequence of human Delta3, the nucleotide at position 786 is an cytosine (C)(SEQ ID NO: 1). In this embodiment, the amino acid at position 150 is a alanine (A)(SEQ ID NO: 2). In an alternative embodiment, a species variant of human Delta3 has a nucleotide at position 786 which is a thymidine (T)(SEQ ID NO: 33). In this embodiment, the amino acid at position 150 is valine (V)(SEQ ID NO: 34), i.e., a conservative substitution.

In one embodiment of a nucleotide sequence of human Delta3, the nucleotide at position 594 is a cytosine (C)(SEQ ID NO: 1). In this embodiment, the amino acid at position 86 is threonine (T)(SEQ ID NO: 2). In an alternative embodiment, a species variant of human Delta3 has a nucleotide at position 594 which is a guanine (G)(SEQ ID NO: 35). In this embodiment, the amino acid at position 86 is serine (S)(SEQ ID NO: 36), i.e., a conservative substitution.

In one embodiment of a nucleotide sequence of human Delta3, wherein the nucleotide at position 883 is a thymidine (T)(SEQ ID NO: 1). In this embodiment, the amino acid at position 182 is aspartate (D)(SEQ ID NO: 2). In an alternative embodiment, a species variant of human Delta3 has a nucleotide at position 883 which is an adenine (A)(SEQ ID NO: 37). In this embodiment, the amino acid at position 182 is glutamate (E)(SEQ ID NO: 38), i.e., a conservative substitution.

In one embodiment of a nucleotide sequence of mouse Delta3, the nucleotide at position 49 is cytosine (C)(SEQ ID NO: 24). In this embodiment, the amino acid at position 4 is alanine (A)(SEQ ID NO: 25). In an alternative embodiment, a species variant of mouse Delta3 has a nucleotide at position 49 which is thymidine (T)(SEQ ID NO: 39). In this embodiment, the amino acid at position 4 is valine (V)(SEQ ID NO: 40), i.e., a conservative substitution.

In one embodiment of a nucleotide sequence of mouse Delta3, the nucleotide at position 51 is thymidine (T)(SEQ ID NO: 24). In this embodiment, the amino acid at position 5 is serine (S)(SEQ ID NO: 25). In an alternative embodiment, a species variant of mouse Delta3 has a nucleotide at position 51 which is a adenine (A)(SEQ ID NO: 41). In this embodiment, the amino acid at position 5 is threonine (T)(SEQ ID NO: 42), i.e., a conservative substitution.

In one embodiment of a nucleotide sequence of mouse Delta3, the nucleotide at position 109 is guanine (G)(SEQ ID NO: 24). In this embodiment, the amino acid at position 24 is arginine (R)(SEQ ID NO: 25). In an alternative embodiment, a species variant of mouse Delta3 has a nucleotide at position 109 which is adenine (A)(SEQ ID NO: 43). In this embodiment, the amino acid at position 24 is histidine (H)(SEQ ID NO: 44), i.e., a conservative substitution.

In one embodiment of a nucleotide sequence of mouse Delta3, wherein the nucleotide at position 130 is a thymidine (T)(SEQ ID NO: 24). In this embodiment, the amino acid at position 31 is phenylalanine (F)(SEQ ID NO: 25). In an alternative embodiment, a species variant of mouse Delta3 has a nucleotide at position 130 which is adenine (A)(SEQ ID NO: 45). In this embodiment, the amino acid at position 31 is tyrosine (Y)(SEQ ID NO: 46), i.e., a conservative substitution.

The invention also pertains to nucleic acid molecules encoding a polypeptide of the invention that contain changes in amino acid residues that are not essential for activity. Such polypeptides differ in amino acid sequence from SEQ ID NOs: 2, 25, 28, 30, 32, 34, 36, 38, 40, 42, 44 or 46, or the amino acid sequence encoded by the cDNA of a clone deposited with the ATCC® as Accession Number 98348 yet retain biological activity. In one embodiment, the isolated nucleic acid molecule includes a nucleotide sequence encoding a protein that includes an amino acid sequence that is at least 65%, 75%, 85%, 95%, or 98% identical to the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348.

Moreover, nucleic acid molecules encoding proteins of the invention from other species (homologs), which have a nucleotide sequence which differs from that of the human protein described herein are intended to be within the scope of the invention. Nucleic acid molecules corresponding to natural allelic variants and homologs of a cDNA of the invention can be isolated based on their identity to the human nucleic acid molecule disclosed herein using the human cDNAs, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 480 (500, 550, 600, 650, 700, 800, 900, 1000, 1100, 1200, 1300, 1400, 1500, 1600, 1700, 1800, 1900, 2000 or 2050) nucleotides in length and hybridizes under stringent conditions to the nucleic acid molecule comprising the nucleotide sequence, preferably the coding sequence, of SEQ ID NOs: 1, 3, 24, 26, 27, 29, 31, 33, 35, 37, 39, 41, 43 or 45, or the nucleotide sequence of the cDNA of a clone deposited with the ATCC® as Accession Number 98348, or complement thereof.

Preferably, such nucleic acid molecules, “specifically hybridize” or “specifically detect” a nucleic acid molecule of the invention by exhibiting the ability to hybridize to at least approximately 6, 12, 20, 30, 50, 100, 150, 200, 300, 350, 400 or 425 consecutive nucleotides of a Delta3 nucleotide sequence designated in one of SEQ ID Nos: 1, 3, 24, 26, 27, 29, 31, 33, 35, 37, 39, 41, 43 or 45, or the nucleotide sequence of the cDNA of a clone deposited with the ATCC® as Accession Number 98348, or a sequence complementary thereto, such that more than 5, 10 or 20 times more hybridization (utilizing hybridization conditions described above), preferably more than 50 times more hybridization, and even more preferably more than 100 times more hybridization than occurs relative to hybridization to a cellular nucleic acid (e.g., mRNA or genomic DNA) encoding a protein other than a Delta3 protein as defined herein.

Delta3 nucleic acids can encode polypeptides that are at least 55% identical to an amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Nucleic acids which encode polypeptides which are at least about 72%, and even more preferably at least about 80%, 85%, 90%, 95%, or 98% similar with an amino acid sequence represented in SEQ ID NO: 2, 25, 28, 30, 32, 34, 36, 38, 40, 42, 44 or 46, or the amino acid sequence encoded by the cDNA of a clone deposited with the ATCC® as Accession Number 98348 are also within the scope of the invention.

In one embodiment, the nucleic acid of the present invention encodes a polypeptide having an overall amino acid sequence similarity of at least about 72%, at least about 80%, at least about 85%, at least about 90%, at least about 95%, at least about 98%, or at least about 99% with the amino acid sequence shown in SEQ ID NOs: 2, 25, 28, 30, 32, 34, 36, 38, 40, 42, 44 or 46. In a preferred embodiment, the nucleic acid encodes a protein comprising the amino acid set forth in SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Preferably, the nucleic acid includes all or a portion of the nucleotide sequence corresponding to the coding region of SEQ ID Nos: 1, 3, 24, 26, 27, 29, 31, 33, 35, 37, 39, 41, 43 or 45, or the nucleotide sequence of the cDNA of a clone deposited with the ATCC® as Accession Number 98348.

The nucleic acids of the invention can encode a Delta3 protein from any species, including insects. Preferred nucleic acids encode vertebrate Delta3 proteins. Even more preferred nucleic acids encode mammalian Delta3 proteins including primate Delta3 proteins, e.g., human Delta3 proteins, and murine Delta3 proteins. Other nucleic acids of the invention can encode avian, equine, canine, feline, bovine or porcine Delta3 proteins.

In a preferred embodiment of the invention, the nucleic acid encodes a polypeptide comprising an extracellular domain of Delta3, e.g., human or mouse Delta3 including allelic variants having SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Accordingly, preferred nucleic acids encode a polypeptide comprising about amino acid 1 to about amino acid 529 of SEQ ID NO: 2, 28, 30, 32, 34, 36 or 38, or alternatively about amino acid 1 to about amino acid 530 of SEQ ID NO: 25, 40, 42, 44, or 46.

Other preferred nucleic acids encode a polypeptide corresponding to an extracellular domain of Delta3 lacking the signal peptide, e.g., a polypeptide comprising about amino acid 18 to about amino acid 529 of SEQ ID NO: 2 or about amino acid 18 to about amino acid 530 of SEQ ID NO: 25. Yet other preferred nucleic acids encode a polypeptide comprising at least one of the conserved motifs in the extracellular domain of Delta3, e.g., a DSL motif (for example, amino acids 173-217 of SEQ ID NO: 2 or amino acids 174-218 of SEQ ID NO: 25) or an EGF-like motif (for example, EGF-like 1, amino acids 222-250 of SEQ ID NO: 2), such as those shown in FIG. 2, or also for example amino acids 223-251 of SEQ ID NO: 25. Additional EGF-like domains are from amino acids 253-281, 288-321, 328-359, 366-399, 411-437, 444-475, and 484-517 of SEQ ID NO: 2 and 254-282, 289-322, 329-360, 367-400, 412-438, 445-476, and 485-518 of SEQ ID NO: 25.

In one embodiment, the nucleic acid encodes a protein having at least one EGF-like motif. In other embodiments, the nucleic acid encodes proteins having at least 2, at least 3, at least 4, at least 5, at least 6, at least 7, or 8 EGF-like domains, such as those shown in boxes in FIG. 1 (SEQ ID NO: 2) or amino acids 223-251, 254-282, 289-322, 329-360, 367-400, 412-438, 445-476, and 485-518 of SEQ ID NO: 25.

The polypeptide encoded by a nucleic acid encoding any of these numbers of EGF-like domains can further comprise an amino acid sequence encoding a DSL motif.

The DSL region or motif is shared by all known members of the family of presumed ligands of Notch-like proteins (Delta1 and Serrate in Drosophila; Lag-2 and Apx-1 in Caenorhabditis elegans) (Henderson et al. (1994) Development 120:2913; Tax et al. (1994) Nature 368:150; Fleming et al. (1990) Genes Dev. 4:2188; Thomas et al. (1991) Development 11:749; Mello et al. (1994) Cell 77:95). The DSL motif is located in the amino terminal portion of the protein, i.e., extracellular, which is closely related to a similar domain in the Drosophila Delta1 protein and which has been described as being necessary and sufficient for in vitro binding to Notch (Henrique et al. (1995) Nature 375:787; Muskavitch (1994) Dev. Biol. 166:415).

In one embodiment, a nucleic acid of the invention encodes a polypeptide that comprises a human or mouse Delta3 DSL domain and which is capable of binding a receptor. A Delta3 DSL domain conforms to the following DSL consensus sequence: X-X-C-X-X-X-Y-[FY]-G-X-X-C-X-X-X-C-[HR]-X-R-X-D-X-F-G-[RH]-X-X-C-X-X-X-G-X-X-X-C-X-X-G-W-X-G-X-Y-C wherein all amino acids are indicated according to their universal single letter designation, brackets indicate that the amino acid at that position is selected from one of the amino acids within the brackets, and “X” designates any amino acid, and wherein the DSL domain is at least 60%, or more preferably at least 65%, 70%, 75%, 80%, 85%, or most preferably 90%, 95%, 98% or 100% identical, i.e., no gaps in the sequence, to the human Delta3 polypeptide sequence from amino acids 173-217 of SEQ ID NO:2. In another embodiment, a Delta3 DSL domain conforms to the above-described DSL consensus sequence and is at least 60%, or more preferably at least 65%, 70%, 75%, 80%, 85%, or most preferably 90%, 95%, 98% or 100% identical to the mouse Delta3 DSL sequence from amino acids 174-218 of SEQ ID NO: 25.

In another embodiment, a nucleic acid of the invention encodes Delta3 DSL domain has a cysteine at amino acid positions 176, 185, 189, 201, 209, and 217 of SEQ ID NO: 2, and is at least 60%, or more preferably at least 65%, 70%, 75%, 80%, 85%, or most preferably 90%, 95%, 98% or 100% identical to the human Delta3 polypeptide sequence from amino acids 173-217 of SEQ ID NO:2. In another embodiment, a Delta3 DSL domain has a cysteine at amino acid positions 177, 186, 190, 202, 210, and 218 of SEQ ID NO: 25, and is at least 60%, or more preferably at least 65%, 70%, 75%, 80%, 85%, or most preferably 90%, 95%, 98% or 100% identical to the mouse Delta3 DSL sequence from amino acids 174-218 of SEQ ID NO: 25.

In one embodiment, a nucleic acid of the invention encodes a polypeptide that comprises a human or mouse Delta3 EGF-like domain. An EGF-like domain has the following consensus sequence: C-X₄₋₈-C-X₁₋₂-G-X-C-X₅₋₉-[WFY]-X-C-X-C-X₂₋₄-G-[WFY]-G-X₁₋₃-[FY]-C, wherein all amino acids are indicated according to their universal single letter designation, brackets indicate that the amino acid at that position is selected from one of the amino acids within the brackets, and “X” designates any amino acid. The numbers in subscript next to an amino acid position indicate a range of possible amino acids, for example, C-X₅₋₉-C indicates that there is a cysteine followed by any 5 to 9 amino acids followed by a cysteine. In one embodiment, an EGF-like domain of the invention is at least 75%, or more preferably at least 80%, 85%, or most preferably 90%, 95%, 98% or 100% identical, i.e., no gaps in the sequence, to the human Delta3 polypeptide sequence from amino acids 222-250, amino acids 253-281, amino acids 288-321, amino acids 328-359, amino acids 366-399, amino acids 411-437, amino acids 444-475, and amino acids 484-517 of SEQ ID NO: 2. In another preferred embodiment, a Delta3 EGF-like domain conforms to the above-described EGF-like consensus sequence and is at least 60%, or more preferably at least 75%, 80%, 85%, or most preferably 90%, 95%, 98% or 100% identical to the mouse Delta3 DSL sequence from amino acids 223-251, amino acids 254-282, amino acids 289-322, amino acids 329-360, amino acids 367-400, amino acids 412-438, amino acids 445-476, and amino acids 485-518 of SEQ ID NO: 25.

In another embodiment, a nucleic acid of the invention encodes Delta3 EGF-like domain is at least 75%, or more preferably at least 80%, 85%, or most preferably 90%, 95%, 98% or 100% identical to the human Delta3 polypeptide sequence from amino acids 222-250, amino acids 253-281, amino acids 288-321, amino acids 328-359, amino acids 366-399, amino acids 411-437, amino acids 444-475, or amino acids 484-517 of SEQ ID NO: 2. In another embodiment, a Delta3 EGF-like domain is at least 75%, or more preferably at least 80%, 85%, or most preferably 90%, 95%, 98% or 100% identical to the mouse Delta3 EGF-like domain sequence from amino acids 223-251, amino acids 254-282, amino acids 289-322, amino acids 329-360, amino acids 367-400, amino acids 412-438, amino acids 445-476, or amino acids 485-518 of SEQ ID NO: 25.

Polypeptides encoded by any of the above-described nucleic acids can be soluble. Preferred soluble peptides comprise at least a portion of the extracellular domain of a Delta3 protein. Even more preferred soluble polypeptides comprise an amino acid sequence corresponding to about amino acid 1 to about amino acid 529 of SEQ ID NO: 2, corresponding to about amino acid 18 to about amino acid 529 of SEQ ID NO: 2 or a homolog thereof. Alternatively, an extracellular domain is comprised of about amino acid 1 to about amino acid 530 of SEQ ID NO: 25, about amino acid 18 to about amino acid 530 of SEQ ID NO: 25 or a homolog thereof.

Yet other preferred soluble Delta3 polypeptides comprise at least one EGF-like domain. Such polypeptides may in addition comprise a DSL domain and optionally a signal peptide.

In another embodiment, nucleic acids encode a Delta3 polypeptide as part of a fusion protein. A preferred fusion protein is a Delta3 Immunoglobulin (Ig) fusion protein, or alternatively, a Delta3 portion as described above fused to Ig. Such fusion proteins can comprise at least a portion of the extracellular domain of a Delta3 domain. A portion can be any portion of at least about 10 amino acids, such as the portions described above. Nucleic acids encoding such fusion proteins can be prepared, e.g., as described in U.S. Pat. No. 5,434,131.

Alternatively, polypeptides encoded by the nucleic acid of the invention can be membrane bound. Membrane bound polypeptides of the invention preferably comprise a transmembrane domain. As used herein, a “transmembrane domain” refers to an amino acid sequence having at least about 20 to 25 amino acid residues in length and which contains at least about 65-70% hydrophobic amino acid residues such as alanine, leucine, isoleucine, phenylalanine, proline, tyrosine, tryptophan, or valine. The transmembrane domain can be from a Delta3 protein, such as a transmembrane domain comprising amino acid 530 to amino acid 553 of SEQ ID NO: 2, shown in FIG. 1 or amino acid 531 to amino acid 554 of SED ID NO: 25, shown in FIG. 4.

In a one embodiment, a nucleic acid of the invention encodes transmembrane domain contains at least about 15 to 30 amino acid residues, preferably about 20-25 amino acid residues, and has at least about 60-80%, more preferably 65-75%, and more preferably at least about 70% hydrophobic residues from about amino acid 530 to about amino acid 553 of SEQ ID NO: 2, shown in FIG. 2 or from about amino acid 531 to about amino acid 554 of SED ID NO: 25.

Alternatively, the transmembrane domain can be from another membrane protein, such as to produce a chimeric membranous Delta3 protein. Yet other polypeptides of the invention can be intracellular proteins. Accordingly, also within the scope of the invention are proteins which do not comprise a transmembrane domain. Other proteins of the invention do not include an extracellular domain. Additional proteins of the invention do not include an extracellular domain nor a transmembrane domain.

Polypeptides encoded by the nucleic acid of the invention can comprise a cytoplasmic domain. In a preferred embodiment, a nucleic acid of the invention encodes a polypeptide comprising a Delta3 cytoplasmic domain. In an even more preferred embodiment, the cytoplasmic domain has an amino acid sequence corresponding to a sequence from about amino acid 554 to about amino acid 685 of SEQ ID NO: 2 (FIG. 1), or a portion thereof, or from about amino acid 555 to about amino acid 686 of SEQ ID NO: 25.

In yet other preferred embodiments, the nucleic acid of the invention encodes a polypeptide comprising at least one domain of a Delta3 protein selected from the group consisting of: a signal peptide, a DSL motif, an EGF-like domain, a transmembrane domain, and a cytoplasmic domain. The polypeptide of the invention can comprise several of these domains from a Delta3 protein.

Alternatively, a nucleic acid of the invention encodes a polypeptide that can be a chimeric protein, i.e., comprised of at least one conserved domain of SEQ ID NO: 2, 25, 2, 25, 28, 30, 32, 34, 36, 38, 40, 42, 44 or 46, or the amino acid sequence encoded by the cDNA of a clone deposited with the ATCC® as Accession Number 98348 and at least one conserved domain from a polypeptide other than a Delta3 protein. Accordingly, in one embodiment, a nucleic acid of the invention encodes a Delta3 polypeptide of 2, 25, 28, 30, 32, 34, 36, 38, 40, 42, 44 or 46, or the amino acid sequence encoded by the cDNA of a clone deposited with the ATCC® as Accession Number 98348 wherein, for example, the DSL motif from amino acids 173-217 of SEQ ID NO: 2, are replaced with amino acids from a comparable DSL domain of a Delta-like protein other than Delta3. Such an amino acid sequence can be any sequence shown in FIG. 1.

In yet another embodiment, the nucleic acid encodes a Delta3 protein having a signal peptide from a protein other than a Delta3 protein. Also within the scope of the invention are Delta3 nucleic acids which encode a Delta3 polypeptide, wherein the cytoplasmic domain is other than a Delta3 cytoplasmic domain. In addition, the invention contemplates a Delta3 nucleic acid molecule, wherein the nucleic acid encodes a Delta3 polypeptide with a cytoplasmic domain and a extracellular domain from a protein other than Delta3.

Delta-like proteins other than Delta3 proteins can be, e.g., toporythmic proteins. “Toporythmic proteins” is intended to include Notch, Delta, Serrate, Enhancer of Split, Deltex, and other members of this family of proteins sharing structural similarities. (See e.g., International Patent Publication Nos. WO 97/01571 (Jan. 16, 1997); WO 92/19734 (Nov. 12, 1992) and WO 94/07474 (Apr. 14, 1994)).

Nucleic acids encoding polypeptides having an amino acid sequence that is homologous to any of the above described portions of SEQ ID NO: 2, 25, 28, 30, 32, 34, 36, 38, 40, 42, 44 or 46, or the amino acid sequence encoded by the cDNA of a clone deposited with the ATCC® as Accession Number 98348 are also within the scope of the invention. Preferred nucleic acids of the invention encode polypeptides comprising an amino acid sequence which are at least about 70%, at least about 75%, at least about 80%, or at least about 85% identical to the amino acid sequence of any of the Delta3 domains shown in FIG. 1. Even more preferred nucleic acids of the invention encode polypeptides comprising an amino acid sequence which are at least about 90%, at least about 95%, at least about 98%, or at least about 99% identical to the amino acid sequence of any of the Delta3 domains shown in FIG. 1.

In one embodiment, the nucleic acid, e.g., cDNA, encodes a peptide having at least one activity of the subject Delta3 polypeptide, such as the ability to bind to a Delta3 interacting molecule, such as a Delta3 receptor e.g., Notch. Non-limiting examples of binding assays for Delta3 interaction with a Delta3 interacting molecule include: measuring interaction of Delta3 polypeptides of the invention with a Delta3 interacting molecule, such as for example Notch, include binding assays involving soluble forms of Delta3 and a Delta3 interacting molecule, measuring a Delta3 domain, e.g., the DSL domain, binding to a Delta3 interacting molecule, measuring Delta3 binding to receptors expressed on cells, and measuring soluble Delta3 binding to an immobilized Delta3 interacting molecule, i.e., solid-phase binding assays. Specific examples of these assays are set forth in Shimizu et al. (1999) J. Biol. Chem. 274:32961-32969.

Additional molecules, e.g., polypeptides or peptides, capable of interacting with a Delta3 protein or fragment thereof can be identified by various methods, e.g., methods based on binding assays. For example, various types of expression libraries can be screened with a Delta3 protein or portion thereof. A two-hybrid system can be used to isolate cytoplasmic proteins interacting with the cytoplasmic domain of Delta3. Portions of Delta3 proteins which are capable of interacting with a ligand can be determined by preparing fragments of Delta3 proteins and screening these fragments for those that are capable of interacting with the ligand.

Based at least in part on the observation that the N-terminal portion of Drosophila Delta protein, which contains a DSL domain and EGF-like domain, is necessary and sufficient for in vitro binding to Notch (Henrique et al. (1995) Nature 375:787; Muskavitch (1994) Dev. Biol. 166:415), it is likely that the domain of Delta3 proteins capable of interacting with a ligand includes the DSL domain and/or at least a portion of the EGF-like domain. However, other portions of the extracellular domain of Delta3 could be necessary for binding to at least some Delta3 ligands.

In other preferred embodiments, the subject Delta3 polypeptide can modulate proliferation and/or differentiation or cell death of specific target cells, e.g., neural cells or endothelial cells. Assays for determining that a Delta3 polypeptide has at least one activity of a Delta3 protein are described infra.

Still other preferred nucleic acids of the present invention encode a Delta3 polypeptide which includes a polypeptide sequence corresponding to all or a portion of amino acid residues in SEQ ID NO: 2, 25, 28, 30, 32, 34, 36, 38, 40, 42, 44 or 46, e.g., at least 2, 5, 10, 25, 50, 100, 150 or 200 amino acid residues of that region. Preferred nucleic acids encode a polypeptide comprising at least two consecutive amino acid residues from about amino acid 1 to about amino acid 570 of the amino acid sequence set forth in SEQ ID NO: 2 or from about amino acid 1 to about amino acid 571 of the amino acid sequence set forth in SEQ ID NO: 25.

Yet other preferred nucleic acids encode a polypeptide comprising at least about 3, at least about 5, at least about 10, at least about 15, at least about 20, or at least about 25 consecutive amino acids from about amino acid 1 to about amino acid 575 set forth in SEQ ID NO: 2, from about amino acid 18 to about amino acid 575 set forth in SEQ ID NO: 2, from about amino acid 1 to about amino acid 576 set forth in SEQ ID NO: 25, or from about amino acid 18 to about amino acid 576 set forth in SEQ ID NO: 25.

The invention further provides for nucleic acids encoding a polypeptide having an amino acid sequence which is at least about 70%, preferably at least about 80%, and most preferably at least about 90% to at least about 10 consecutive amino acids set forth in SEQ ID NOs: 2, 25, 28, 30, 32, 34, 36, 38, 40, 42, 44 or 46, or at least about 10 consecutive amino acids from a portion of SEQ ID NOs: 2, 25, 28, 30, 32, 34, 36, 38, 40, 42, 44 or 46. In one embodiment, the portion corresponds to about amino acid 1 to about amino acid 575 of SEQ ID NO: 2, about amino acid 18 to about amino acid 575 of SEQ ID NO: 2, from about amino acid 1 to about amino acid 576 set forth in SEQ ID NO: 25, or from about amino acid 1 to about amino acid 576 set forth in SEQ ID NO: 25. Coding nucleic acid molecules of the invention preferably comprise at least about 200, 250, 300, 350, 400, 410, 420, 430, 435 or 440 base pairs.

The invention further pertains to nucleic acid molecules for use as probes/primer or antisense molecules (i.e. non-coding nucleic acid molecules), which can comprise at least about 6, 12, 20, 30, 50, 100, 125, 150 or 200 nucleotides or base pairs. Yet other preferred nucleic acids of the invention comprise at least about 300, at least about 350, at least about 400, at least about 450, at least about 500, or at least about 600 nucleotides of SEQ ID NOs: 1, 3, 24, 26, 27, 29, 31, 33, 35, 37, 39, 41, 43 or 45. In some embodiments, the nucleic acids of the invention correspond to a 5′ portion of nucleic acid sequence SEQ ID NO: 1, 3, 24, 26, 27, 29, 31, 33, 35, 37, 39, 41, 43 or 45. For example, a nucleic acid of the invention can correspond to a portion of about nucleotide 1 to about nucleotide 2000 of nucleic acid sequence SEQ ID NO: 1, 3, 24, 26, 27, 29, 31, 33, 35, 37, 39, 41, 43 or 45.

Preferred nucleic acids for use as a probe according to the methods of the invention include nucleic acids comprising a nucleotide sequence having at least about 6, preferably at least about 9, more preferably at least about 12 and even more preferably at least about 15 consecutive nucleotides from SEQ ID NOs: Nos: 1, 3, 24, 26, 27, 29, 31, 33, 35, 37, 39, 41, 43 or 45, or from a portion thereof. In a preferred embodiment, the portion corresponds to about nucleotide 1 to about nucleotide 2060 of SEQ ID NOs: Nos: 1, 3, 24, 26, 27, 29, 31, 33, 35, 37, 39, 41, 43 or 45. Alternatively a portion can be a nucleotide sequence encoding a conserved motif of hDelta3 or mDelta3 protein. Alternatively, the portion can be a nucleotide sequence located between nucleic acid sequences encoding conserved motifs of a human or mouse Delta3 protein.

The invention further provides for a combination of at least two nucleic acids corresponding to at least a portion of SEQ ID NOs: Nos: 1, 3, 24, 26, 27, 29, 31, 33, 35, 37, 39, 41, 43 or 45, or a homolog thereof. Accordingly, in one embodiment, the invention provides a combination of two nucleic acids of at least about 6, preferably at least about 9, more preferably at least about 12 and even more preferably at least about 15 consecutive nucleotides from SEQ ID NOs: Nos:1, 3, 24, 26, 27, 29, 31, 33, 35, 37, 39, 41, 43 or 45, or from a portion thereof. In a preferred embodiment, at least one of the nucleic acids is labeled.

Another aspect of the invention provides a nucleic acid which hybridizes under stringent conditions to a nucleic acid represented by one of SEQ ID Nos:1, 3, 24, 26, 27, 29, 31, 33, 35, 37, 39, 41, 43 or 45. Appropriate stringency conditions which promote DNA hybridization, for example, 6.0× sodium chloride/sodium citrate (SSC) at about 45□C., followed by a wash of 2.0× SSC at 50□C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0× SSC at 50□C. to a high stringency of about 0.2× SSC at 50□C. or at 65□C. In addition, the temperature in the wash step can be increased from low stringency conditions at room temperature, about 22□C., to high stringency conditions at about 65□C. Both temperature and salt may be varied, or temperature of salt concentration may be held constant while the other variable is changed.

In yet another embodiment, a naturally occurring Delta3 nucleic acid of the invention, e.g., SEQ ID NO: 1, 3, 24 or 26 hybridizes under high stringency conditions to a species variant of Delta3, such as a species variant shown in any one of SEQ ID NOs: 27, 29, 31, 33, 35, 37, 39, 41, 43 or 45. In yet another embodiment, a Delta3 nucleic acid, e.g., SEQ ID NO: 1, 3, 24, 26, 27, 29, 31, 33, 35, 37, 39, 41, 43 or 45 hybridizes under high stringency conditions to a representative mammalian Delta3.

Preferred nucleic acids have a sequence at least about 75% identical and more preferably at least about 80% and even more preferably at least about 85% identical with a nucleic acid sequence of a Delta3 gene, such as a human Delta3 gene or a mouse Delta3 gene, e.g., such as a sequence shown in one of SEQ ID NOs: 1, 3, 24, 26, 27, 29, 31, 33, 35, 37, 39, 41, 43 or 45. Nucleic acids at least about 90%, more preferably at least about 95%, and most preferably at least about 98-99% homologous with a nucleic sequence represented in one of SEQ ID NOs: 1, 3, 24, 26, 27, 29, 31, 33, 35, 37, 39, 41, 43 or 45 are of course also within the scope of the invention. In preferred embodiments, the nucleic acid is a human Delta3 gene and in particularly preferred embodiments, includes all or a portion of the nucleotide sequence corresponding to the coding region of one of SEQ ID NOs: 1, 3, 24, 26, 27, 29, 31, 33, 35, 37, 39, 41, 43 or 45.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered, individually or in combination (that is two, threee, four, or more different antisense molecules), to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In various embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996) Bioorganic & Medicinal Chemistry 4(1): 5-23; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup et al. (1996) Bioorganic & Medicinal Chemistry 4(1): 5-23; or as probes or primers for DNA sequence and hybridization (Hyrup et al. (1996) Bioorganic & Medicinal Chemistry 4(1): 5-23; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup et al. (1996) Bioorganic & Medicinal Chemistry 4(1): 5-23). The synthesis of PNA-DNA chimeras can be performed as described in Hyrup et al. (1996) Bioorganic & Medicinal Chemistry 4(1): 5-23, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication NO: WO 88/09810) or the blood-brain barrier (see, e.g., PCT Publication NO: WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

Nucleic acids having a sequence that differs from the nucleotide sequences shown in one of SEQ ID Nos: 1, 3, 24, 26, 27, 29, 31, 33, 35, 37, 39, 41, 43 or 45 due to degeneracy in the genetic code are also within the scope of the invention. Such nucleic acids encode functionally equivalent peptides (i.e., a peptide having a biological activity of a Delta3 polypeptide) but differ from the sequence shown in the sequence listing due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence of a Delta3 polypeptide. However, it is expected that DNA sequence polymorphisms that do lead to changes in the amino acid sequences of the subject Delta3 polypeptides will exist. One skilled in the art will appreciate that these variations in one or more nucleotides (e.g., up to about 3-5% of the nucleotides) of the nucleic acids encoding polypeptides having an activity of a Delta3 polypeptide may exist among individuals of a given species due to natural allelic variation.

As indicated by the examples set out below, Delta3 protein-encoding nucleic acids can be obtained from mRNA present in any of a number of eukaryotic cells. It should also be possible to obtain nucleic acids encoding Delta3 polypeptides of the present invention from genomic DNA from both adults and embryos. For example, a gene encoding a Delta3 protein can be cloned from either a cDNA or a genomic library in accordance with protocols described herein, as well as those generally known to persons skilled in the art. Examples of tissues and/or libraries suitable for isolation of the subject nucleic acids include endothelial cell libraries, among others. A cDNA encoding a Delta3 protein can be obtained by isolating total mRNA from a cell, e.g., a vertebrate cell, a mammalian cell, or a human cell, including embryonic cells. Double stranded cDNAs can then be prepared from the total mRNA, and subsequently inserted into a suitable plasmid or bacteriophage vector using any one of a number of known techniques. The gene encoding a Delta3 protein can also be cloned using established polymerase chain reaction techniques in accordance with the nucleotide sequence information provided by the invention. The nucleic acid of the invention can be DNA or RNA. A preferred nucleic acid is a cDNA represented by a sequence selected from the group consisting of SEQ ID NOs: 1, 3, 24, 26, 27, 29, 31, 33, 35, 37, 39, 41, 43 or 45.

4.2.1. Vectors

This invention also provides expression vectors containing a nucleic acid encoding a Delta3 polypeptide, operably linked to at least one transcriptional regulatory sequence. “Operably linked” is intended to mean that the nucleotide sequence is linked to a regulatory sequence in a manner which allows expression of the nucleotide sequence. Regulatory sequences are art-recognized and are selected to direct expression of the subject Delta3 proteins. Accordingly, the term “transcriptional regulatory sequence” includes promoters, enhancers and other expression control elements. Such regulatory sequences are described in Goeddel; Gene Expression Technology: Methods in Enzymology 185, Academic Press, San Diego, Calif. (1990). In one embodiment, the expression vector includes a recombinant gene encoding a peptide having an agonistic activity of a subject Delta3 polypeptide, or alternatively, encoding a peptide which is an antagonistic form of the Delta3 protein. Such expression vectors can be used to transfect cells and thereby produce polypeptides, including fusion proteins, encoded by nucleic acids as described herein. Moreover, the gene constructs of the present invention can also be used as a part of a gene therapy protocol to deliver nucleic acids encoding either an agonistic or antagonistic form of one of the subject Delta3 proteins. Thus, another aspect of the invention features expression vectors for in vivo or in vitro transfection and expression of a Delta3 polypeptide in particular cell types so as to reconstitute the function of, or alternatively, abrogate the function of Delta-induced signaling in a tissue. This could be desirable, for example, when the naturally-occurring form of the protein is expressed inappropriately; or to deliver a form of the protein which alters differentiation of tissue. Expression vectors may also be employed to inhibit neoplastic transformation.

In addition to viral transfer methods, such as those illustrated above, non-viral methods can also be employed to cause expression of a subject Delta3 polypeptide in the tissue of an animal. Most nonviral methods of gene transfer rely on normal mechanisms used by mammalian cells for the uptake and intracellular transport of macromolecules. In preferred embodiments, non-viral targeting means of the present invention rely on endocytic pathways for the uptake of the subject Delta3 polypeptide gene by the targeted cell. Exemplary targeting means of this type include liposomal derived systems, polylysine conjugates, and artificial viral envelopes.

4.2.2. Probes and Primers

Moreover, the nucleotide sequences determined from the cloning of hDelta3 genes will further allow for the generation of probes and primers designed for use in identifying and/or cloning Delta3 homologs in other cell types, e.g., from other tissues, as well as Delta3 homologs from other mammalian organisms. Probes and primers of the invention can also be used to determine the identity of a Delta3 allele and/or the presence or absence of one or more mutations in a Delta3 gene of a subject. In a preferred embodiment, a probe or primer of the invention can be used to determine whether a subject has or is at risk of developing a disease or condition associated with a specific Delta3 allele, such as an allele carrying a mutation.

In a preferred embodiment, the present invention also provides a probe/primer comprising a substantially purified oligonucleotide, which oligonucleotide comprises a region of nucleotide sequence that hybridizes under stringent conditions to at least about 12, preferably about 25, more preferably about 40, 50 or 75 consecutive nucleotides of sense or anti-sense sequence selected from the group consisting of SEQ ID NO: 1, 3, 24, 26, 27, 29, 31, 33, 35, 37, 39, 41, 43 or 45, or naturally-occurring mutants thereof. For instance, primers based on the nucleic acid represented in SEQ ID NOs: 1, 3, 24, 26, 27, 29, 31, 33, 35, 37, 39, 41, 43 or 45 can be used in PCR reactions to clone Delta3 homologs, e.g., specific Delta3 alleles. Such primers are preferably selected in a region which does not share significant homology to other genes, e.g., other Delta genes. Examples of primers of the invention are set forth as SEQ ID NOs: 12-15, set forth below:

| | |
|---|---|
| 5′ end primers: | |
| 5′ AGCGCCTCTGGCTGGGCGCT 3′; | (SEQ ID NO: 12; corresponding to nucleotides 356 to 375 of SEQ ID NO: 1) |
| 5′ CGGCCAGAGGCCTTGCCACC 3′; | (SEQ ID NO: 13; corresponding to nucleotides 725 to 744 of SEQ ID NO: 1) |
| 3′ end primers: | |
| 5′ TTGCGCTCCCGGCTGGAGCC 3′; and | (SEQ ID NO: 14; corresponding to the complement of nucleotides 1460 to |
| | 1479 of SEQ ID NO: 1) |
| 5′ ATGCGGCTTGGACCTCGGTT 3′. | (SEQ ID NO: 15; corresponding to the complement of nucleotides 1592 to |
| | 2611 of SEQ ID NO: 1) |

Likewise, probes based on the subject Delta3 sequences can be used to detect transcripts or genomic sequences encoding the same or homologous proteins. In preferred embodiments, the probe further comprises a label group attached thereto and able to be detected, e.g., the label group is selected from amongst radioisotopes, fluorescent compounds, enzymes, and enzyme co-factors.

As discussed in more detail below, such probes can also be used as a part of a diagnostic test kit for identifying cells or tissue which misexpress a Delta3 protein, such as by measuring a level of a Delta-encoding nucleic acid in a sample of cells from a patient; e.g., detecting Delta3 mRNA levels or determining whether a genomic Delta3 gene has been mutated or deleted. Briefly, nucleotide probes can be generated from the subject Delta3 genes which facilitate histological screening of intact tissue and tissue samples for the presence (or absence) of Delta-encoding transcripts. Similar to the diagnostic uses of anti-Delta3 antibodies, the use of probes directed to Delta3 messages, or to genomic Delta3 sequences, can be used for both predictive and therapeutic evaluation of allelic mutations which might be manifest in, for example, neoplastic or hyperplastic disorders (e.g., unwanted cell growth) or abnormal differentiation of tissue. Used in conjunction with immunoassays as described herein, the oligonucleotide probes can help facilitate the determination of the molecular basis for a developmental disorder which may involve some abnormality associated with expression (or lack thereof) of a Delta3 protein. For instance, variation in polypeptide synthesis can be differentiated from a mutation in a coding sequence.

Also within the scope of the invention are kits for determining whether a subject is at risk of developing a disease or condition caused by or contributed by an aberrant Delta3 activity and/or which is associated with one or more specific Delta3 alleles. In a preferred embodiment, the kit can be used for determining whether a subject is at risk of developing a neurological disease or disorder, e.g., a peripheral neuropathy, e.g., ACCPN.

4.2.3. Antisense, Ribozyme and Triplex Techniques

One aspect of the invention relates to the use of the isolated nucleic acid in “antisense” therapy. As used herein, “antisense” therapy refers to administration or in situ generation of oligonucleotide molecules or their derivatives which specifically hybridize (e.g., bind) under cellular conditions, with the cellular mRNA and/or genomic DNA encoding one or more of the subject Delta3 proteins so as to inhibit expression of that protein, e.g., by inhibiting transcription and/or translation. The binding may be by conventional base pair complementarity, or, for example, in the case of binding to DNA duplexes, through specific interactions in the major groove of the double helix. In general, “antisense” therapy refers to the range of techniques generally employed in the art, and includes any therapy which relies on specific binding to oligonucleotide sequences.

An antisense construct of the present invention can be delivered, for example, as an expression plasmid which, when transcribed in the cell, produces RNA which is complementary to at least a unique portion of the cellular mRNA which encodes a Delta3 protein. Alternatively, the antisense construct is an oligonucleotide probe which is generated ex vivo and which, when introduced into the cell causes inhibition of expression by hybridizing with the mRNA and/or genomic sequences of a Delta3 gene. Such oligonucleotide probes are preferably modified oligonucleotides which are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668.

With respect to antisense DNA, oligodeoxyribonucleotides derived from the translation initiation site, i.e., the ATG codon which encodes the first methionine of the cDNA, e.g., between the −10 and +10 regions of the Delta3 nucleotide sequence of interest, are preferred. Preferred antisense molecules of the invention are from nucleotides 328 to 348 of SEQ ID NO: 1 or nucleotides 38 to 58 of SEQ ID NO: 24. Non-limiting examples of preferred human and mouse antisense primers are shown below:

| (SEQ ID NO: 16) | |
|---|---|
| 5′ TGCCGCCATCCCTCGGGGCGT 3′ | (complement to nucleotides 326-346 of SEQ ID NO: 1) |
| (SEQ ID NO: 17) | |
| 5′ GGACGCTGCCGCCATCCCCT 3′ | (complement to nucleotides 333-352 of SEQ ID NO: 1) |
| (SEQ ID NO: 18) | |
| 5′ GGACGCTGCCGCCATCCCCTCGGGGCGT 3′ | (complement to nucleotides 326-352 of SEQ ID NO: 1) |
| (SEQ ID NO: 47) | |
| 5′ CTCCGGGACGCAGGCGTCATCCCT 3′ | (complement to nucleotides 38-58 of SEQ ID NO: 24) |
| (SEQ ID NO: 48) | |
| 5′ ACAGGCGCTCCGGGACGCAGGCGTCATCC 3′ | (complement to nucleotides 40-65 of SEQ ID NO: 24) |

Antisense approaches involve the design of oligonucleotides (either DNA or RNA) that are complementary to Delta3 mRNA. The antisense oligonucleotides will bind to the Delta3 mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well. (Wagner, R. (1994) Nature 372:333). Therefore, oligonucleotides complementary to either the 5′ or 3′ untranslated, non-coding regions of a Delta3 gene could be used in an antisense approach to inhibit translation of endogenous Delta3 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′,3′ or coding region of Delta3 mRNA, antisense nucleic acids should be at least six nucleotides in length, and are preferably oligonucleotides ranging from 6 to about 50 nucleotides in length. In certain embodiments, the oligonucleotide is at least 10 nucleotides, at least 17 nucleotides, at least 25 nucleotides, or at least 50 nucleotides.

Regardless of the choice of target sequence, it is preferred that in vitro studies are first performed to quantitate the ability of the antisense oligonucleotide to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication NO: WO 88/09810, Dec. 15, 1988) or the blood-brain barrier (see, e.g., PCT Publication NO: WO 89/10134, Apr. 25, 1988), hybridization-triggered cleavage agents. (See, e.g., Krol et al. (1988) BioTechniques 6:958-976) or intercalating agents. (See, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an α-anomeric oligonucleotide. An α-anomeric oligonucleotide forms specific double-stranded hybrids with complementary RNA in which, contrary to the usual β-units, the strands run parallel to each other (Gautier et al. (1987) Nucl. Acids Res. 15:6625-6641). The oligonucleotide is a 2′-0-methylribonucleotide (Inoue et al. (1987) Nucl. Acids Res. 15:6131-6148), or a chimeric RNA-DNA analogue (Inoue et al. (1987) FEBS Lett. 215:327-330).

Oligonucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al. (1988) Proc. Natl. Acad. Sci. U.S.A. 85:7448-7451), etc.

While antisense nucleic acids complementary to the coding region sequence could be used, those complementary to the transcribed untranslated region are preferred. Antisense nucleic acids overlapping the site of initiation of translation are even more preferred. For example, antisense oligonucleotides as set forth below can be utilized in accordance with the invention.

| | | |
|---|---|---|
| 5′ TCAATCTGGCTCTGTTCGCG 3′ | (complement to nucleotides 284-303 of SEQ ID NO: 1) | (SEQ ID NO: 19) |
| 5′ CGCTCTCTCCACCCGCGGGCCCTCAA 3′ | (complement to nucleotides 300-325 of SEQ ID NO: 1) | (SEQ ID NO: 20) |
| 5′ GGTGTCCTCTCCACCGGACGCGTGGG 3′ | (complement to nucleotides 6-31 of SEQ ID NO: 24) | (SEQ ID NO: 49) |
| 5′ GTCCTCTCCACCGGACGCGTGG 3′ | (complement to nucleotides 6-28 of SEQ ID NO: 24) | (SEQ ID NO: 50) |

The antisense molecules should be delivered to cells which express the Delta3 in vivo. A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) an be administered systematically.

However, it is often difficult to achieve intracellular concentrations of the antisense sufficient to suppress translation on endogenous mRNAs. Therefore a preferred approach utilizes a recombinant DNA construct in which the antisense oligonucleotide is placed under the control of a strong pol III or pol II promoter. The use of such a construct to transfect target cells in the patient will result in the transcription of sufficient amounts of single stranded RNAs that will form complementary base pairs with the endogenous Delta3 transcripts and thereby prevent translation of the Delta3 mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon (1981) Nature 290:304-310), the promoter contained in the 3′ long terminal repeat of Rous sarcoma virus (Yamamoto et al. (1980) Cell 22:787-797), the herpes thymidine kinase promoter (Wagner et al. (1981) Proc. Natl. Acad. Sci. U.S.A. 78:1441-1445), the regulatory sequences of the metallothionein gene (Brinster et al. (1982) Nature 296:39-42), etc. Any type of plasmid, cosmid, YAC or viral vector can be used to prepare the recombinant DNA construct which can be introduced directly into the tissue site; e.g., the choroid plexus or hypothalamus. Alternatively, viral vectors can be used which selectively infect the desired tissue; (e.g., for brain, herpesvirus vectors may be used), in which case administration may be accomplished by another route (e.g., systematically).

Likewise, the antisense constructs of the present invention, by antagonizing the normal biological activity of one of the Delta3 proteins, can be used in the modulation of cellular activity both in vivo and for ex vivo tissue cultures.

Furthermore, the anti-sense techniques (e.g., microinjection of antisense molecules, or transfection with plasmids whose transcripts are anti-sense with regard to a Delta3 mRNA or gene sequence) can be used to investigate the role of Delta3 in developmental events, as well as the normal cellular function of Delta3 in adult tissue. Such techniques can be utilized in cell culture, but can also be used in the creation of transgenic animals, as detailed below.

Ribozyme molecules designed to catalytically cleave Delta3 mRNA transcripts can also be used to prevent translation of mRNA and expression of Delta3. (See, e.g., PCT International Publication WO 94/11364, published Oct. 4, 1990; Sarver et al., 1990, Science 247:1222-1225). Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage. The composition of ribozyme molecules must include one or more sequences complementary to the target gene mRNA, and must include the well known catalytic sequence responsible for mRNA cleavage. For this sequence, see U.S. Pat. No. 5,093,246. As such within the scope of the invention are engineered hammerhead motif ribozyme molecules that specifically and efficiently catalyze endonucleolytic cleavage of RNA sequences encoding Delta3 proteins.

Specific ribozyme cleavage sites within any potential RNA target are initially identified by scanning the molecule of interest for ribozyme cleavage sites which include the following sequences, GUA, GUU and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate sequences may also be evaluated by testing their accessibility to hybridization with complementary oligonucleotides, using ribonuclease protection assays.

While ribozymes that cleave mRNA at site specific recognition sequences can be used to destroy Delta3 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach (1988) Nature 334:585-591. There are hundreds of potential hammerhead ribozyme cleavage sites within the nucleotide sequence of human Delta3 cDNA (FIG. 1). Preferably the ribozyme is engineered so that the cleavage recognition site is located near the 5′ end of the Delta3 mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al. (1984) Science, 224:574-578; Zaug and Cech (1986) Science, 231:470-475; Zaug, et al. (1986) Nature 324:429-433; PCT Publication WO 88/04300; Been & Cech (1986) Cell 47:207-216). The Cech-type ribozymes have an eight base pair active site which hybridizes to a target RNA sequence whereafter cleavage of the target RNA takes place. The invention encompasses those Cech-type ribozymes which target eight base-pair active site sequences that are present in Delta3.

As in the antisense approach, the ribozymes can be composed of modified oligonucleotides (e.g., for improved stability, targeting, etc.) and should be delivered to cells which express the Delta3 in vivo e.g., hypothalamus and/or the choroid plexus. A preferred method of delivery involves using a DNA construct “encoding” the ribozyme under the control of a strong constitutive pol III or pol II promoter, so that transfected cells will produce sufficient quantities of the ribozyme to destroy endogenous Delta3 messages and inhibit translation. Because ribozymes unlike antisense molecules, are catalytic, a lower intracellular concentration is required for efficiency.

Endogenous Delta3 gene expression can also be reduced by inactivating or “knocking out” the Delta3 gene or its promoter using targeted homologous recombination. (e.g., see Smithies et al. (1985) Nature 317:230-234; Thomas & Capecchi (1987) Cell 51:503-512; Thompson et al. (1989) Cell 5:313-321). For example, a mutant, non-functional Delta3 (or a completely unrelated DNA sequence) flanked by DNA homologous to the endogenous Delta3 gene (either the coding regions or regulatory regions of the Delta3 gene) can be used, with or without a selectable marker and/or a negative selectable marker, to transfect cells that express Delta3 in vivo. Insertion of the DNA construct, via targeted homologous recombination, results in inactivation of the Delta3 gene. Such approaches are particularly suited in the agricultural field where modifications to ES (embryonic stem) cells can be used to generate animal offspring with an inactive Delta3 (e.g., see Thomas & Capecchi (1987) and Thompson (1989), supra). However this approach can be adapted for use in humans provided the recombinant DNA constructs are directly administered or targeted to the required site in vivo using appropriate viral vectors, e.g., herpes virus vectors.

Alternatively, endogenous Delta3 gene expression can be reduced by targeting deoxyribonucleotide sequences complementary to the regulatory region of the Delta3 gene (i.e., the Delta3 promoter and/or enhancers) to form triple helical structures that prevent transcription of the Delta3 gene in target cells in the body. (See generally, Helene, C. (1991) Anticancer Drug Des. 6(6):569-84; Helene, C., et al. (1992) Ann. N.Y. Acad. Sci. 660:27-36; and Maher, L. J. (1992) Bioassays 14(12):807-15).

Nucleic acid molecules to be used in triple helix formation for the inhibition of transcription are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

Antisense RNA and DNA, ribozyme, and triple helix molecules of the invention may be prepared by any method known in the art for the synthesis of DNA and RNA molecules. These include techniques for chemically synthesizing oligo-deoxyribonucleotides and oligoribonucleotides well known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′O-methyl rather than phosphodiesterase linkages within the oligodeoxyribonucleotide backbone.

4.3 Polypeptides of the Present Invention

The present invention also makes available Delta3 polypeptides which are isolated from, or otherwise substantially free of other cellular proteins, especially other signal transduction factors and/or transcription factors which may normally be associated with the Delta3 polypeptide. In general, polypeptides of the invention exhibit an activity of a Delta3 protein. The invention provides various forms of Delta3 proteins, specifically including all of the Delta3 proteins encoded by a nucleic acid of the invention, as described above.

In one embodiment, a polypeptide of the invention is a polypeptide that is a variant of a polypeptide of the invention can be assayed for: (1) the ability to form protein:protein interactions with proteins in a signaling pathway of the polypeptide of the invention; (2) the ability to bind a ligand of the polypeptide of the invention; or (3) the ability to bind to an intracellular target protein of the polypeptide of the invention. In yet another preferred embodiment, the mutant polypeptide can be assayed for the ability to modulate cellular proliferation, cellular migration or chemotaxis, or cellular differentiation.

Full-length proteins or fragments corresponding to one or more particular motifs and/or domains or to arbitrary sizes, for example, at least about 5, 10, 25, 50, 75, 100, 125, 150 amino acids in length are within the scope of the present invention. The invention encompasses all proteins encoded by the nucleic acids described in the above section describing the nucleic acids of the invention.

For example, isolated Delta3 polypeptides can include all or a portion of an amino acid sequences corresponding to a Delta3 polypeptide represented in SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Isolated portions of Delta3 proteins can be obtained, for example, by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a Delta3 polypeptide of the present invention may be divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a wild-type (e.g., “authentic”) Delta3 protein.

In one embodiment, the Delta3 polypeptide of the invention has an overall amino acid sequence similarity or identity of at least about 70%, at least about 75%, at least about 80%, at least about 85%, at least about 90%, at least about 95%, at least about 98%, or at least about 99% with the amino acid sequence SEQ ID NOs: 2, 25, 28, 30, 32, 34, 36, 38, 40, 42, 44 or 46, or the amino acid sequence encoded by the cDNA of a clone deposited with the ATCC® as Accession Number 98348. In a particularly preferred embodiment a Delta3 protein has the amino acid sequence SEQ ID NOs: 2, 25, 28, 30, 32, 34, 36, 38, 40, 42, 44 or 46, or the amino acid sequence of the cDNA of a clone deposited with the ATCC® as Accession Number 98348. In other particularly preferred embodiments, the Delta3 protein has a Delta3 activity.

The present invention further pertains to forms of one of the subject Delta3 polypeptides which are encoded by nucleotide sequences derived from a mammalian organism, and which have amino acid sequences evolutionarily related to the Delta3 protein represented in SEQ ID NOs: 2, 25, 28, 30, 32, 34, 36, 38, 40, 42, 44 or 46, or the amino acid sequence encoded by the cDNA of a clone deposited with the ATCC® as Accession Number 98348. Such recombinant Delta3 polypeptides can, in certain embodiments, preferably are capable of functioning in one of either role of an agonist or antagonist of at least one biological activity of a wild-type (“authentic”) Delta3 protein of the appended sequence listing. The term “evolutionarily related to”, with respect to amino acid sequences of human Delta3 proteins, refers to both polypeptides having amino acid sequences which have arisen naturally, and also to mutational variants of the Delta3 polypeptides which are derived, for example, by combinatorial mutagenesis. Such evolutionarily derived Delta3 polypeptides preferred by the present invention have a Delta3 activity and are at least 80% homologous and more preferably 85% identical and most preferably 90% identical with the amino acid sequence of SEQ ID NOs: 2, 25, 28, 30, 32, 34, 36, 38, 40, 42, 44 or 46, or the amino acid sequence encoded by the cDNA of a clone deposited with the ATCC® as Accession Number 98348. In a particularly preferred embodiment, a Delta3 protein comprises the amino acid coding sequence of SEQ ID NOs: 2, 25, 28, 30, 32, 34, 36, 38, 40, 42, 44 or 46, or the amino acid sequence of the cDNA of a clone deposited with the ATCC® as Accession Number 98348.

The present invention further pertains to methods of producing the subject Delta3 polypeptides. For example, a host cell transfected with a nucleic acid vector directing expression of a nucleotide sequence encoding the subject polypeptides can be cultured under appropriate conditions to allow expression of the peptide to occur. The cells may be harvested, lysed and the protein isolated. A cell culture includes host cells, media and other byproducts. Suitable media for cell culture are well known in the art. The recombinant Delta3 polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In a preferred embodiment, the recombinant Delta3 polypeptide is a fusion protein containing a domain which facilitates its purification, such as GST fusion protein or poly(His) fusion protein.

Moreover, it will be generally appreciated that, under certain circumstances, it may be advantageous to provide variants of one of the subject Delta3 polypeptides which function in a limited capacity as one of either a Delta3 agonist (mimetic) or a Delta3 antagonist, in order to promote or inhibit only a subset of the biological activities of the naturally-occurring form of the protein. Thus, specific biological effects can be elicited by treatment with a variant having a limited function, and with fewer side effects relative to treatment with agonists or antagonists which are directed to all of the biological activities of naturally-occurring forms of Delta3 proteins.

Variants and/or mutants of each of the subject Delta3 proteins can be generated by mutagenesis, such as by discrete point mutation(s), or by truncation. For instance, mutation can give rise to homologs which retain substantially the same, or merely a subset, of the biological activity of the Delta3 polypeptide from which it was derived. Alternatively, antagonistic forms of the protein can be generated which are able to inhibit the function of the naturally-occurring form of the protein, such as by competitively binding to a downstream or upstream member of the Delta3 cascade which includes the Delta3 protein. In addition, agonistic forms of the protein may be generated which are constitutively active.

The recombinant Delta3 polypeptides of the present invention also include homologs of the authentic Delta3 proteins, such as versions of those protein which are resistant to proteolytic cleavage, as for example, due to mutations which alter ubiquitination or other enzymatic targeting associated with the protein.

Delta3 polypeptides may also be chemically modified to create Delta3 derivatives by forming covalent or aggregate conjugates with other chemical moieties, such as glycosyl groups, lipids, phosphate, acetyl groups and the like. Covalent derivatives of Delta3 proteins can be prepared by linking the chemical moieties to functional groups on amino acid sidechains of the protein or at the N-terminus or at the C-terminus of the polypeptide.

Modification of the structure of the subject Delta3 polypeptides can be for such purposes as enhancing therapeutic or prophylactic efficacy, stability (e.g., ex vivo shelf life and resistance to proteolytic degradation in vivo), or post-translational modifications (e.g., to alter phosphorylation pattern of protein). Such modified peptides, when designed to retain at least one activity of the naturally-occurring form of the protein, or to produce specific antagonists thereof, are considered functional equivalents of the Delta3 polypeptides described in more detail herein. Such modified peptides can be produced, for instance, by amino acid substitution, deletion, or addition.

For example, it is reasonable to expect that an isolated replacement of a leucine with an isoleucine or valine, an aspartate with a glutamate, a threonine with a serine, or a similar replacement of an amino acid with a structurally related amino acid (i.e. isosteric and/or isoelectric mutations) will not have a major effect on the biological activity of the resulting molecule. Conservative replacements are those that take place within a family of amino acids that are related in their side chains. Genetically encoded amino acids are can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. In similar fashion, the amino acid repertoire can be grouped as (1) acidic=aspartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (See, for example, Biochemistry, 4th ed., Ed. by L. Stryer, W H Freeman and Co.: 1995). Whether a change in the amino acid sequence of a peptide results in a functional Delta3 homolog (e.g., functional in the sense that the resulting polypeptide mimics or antagonizes the wild-type form) can be readily determined by assessing the ability of the variant peptide to produce a response in cells in a fashion similar to the wild-type protein, or competitively inhibit such a response. Polypeptides in which more than one replacement has taken place can readily be tested in the same manner.

This invention further contemplates a method for generating sets of combinatorial mutants of the subject Delta3 proteins as well as truncation mutants, and is especially useful for identifying potential functional variant sequences (e.g., homologs). The purpose of screening such combinatorial libraries is to generate, for example, novel Delta3 homologs which can act as either agonists or antagonist, or alternatively, possess novel activities all together.

In one embodiment, the variegated library of Delta3 variants is generated by combinatorial mutagenesis at the nucleic acid level, and is encoded by a variegated gene library. For instance, a mixture of synthetic oligonucleotides can be enzymatically ligated into gene sequences such that the degenerate set of potential Delta3 sequences are expressible as individual polypeptides, or alternatively, as a set of larger fusion proteins (e.g., for phage display) containing the set of Delta3 sequences therein.

There are many ways by which such libraries of potential Delta3 variants can be generated from a degenerate oligonucleotide sequence. Chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential Delta3 sequences. The synthesis of degenerate oligonucleotides is well known in the art (see for example, Narang, S A (1983) Tetrahedron 39:3; Itakura et al. (1981) Recombinant DNA, Proc. 3rd Cleveland Sympos. Macromolecules, ed. A G Walton, Amsterdam: Elsevier ppg. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Likewise, a library of coding sequence fragments can be provided for a Delta3 clone in order to generate a variegated population of Delta3 fragments for screening and subsequent selection of bioactive fragments. A variety of techniques are known in the art for generating such libraries, including chemical synthesis. In one embodiment, a library of coding sequence fragments can be generated by (i) treating a double stranded PCR fragment of a Delta3 coding sequence with a nuclease under conditions wherein nicking occurs only about once per molecule; (ii) denaturing the double stranded DNA; (iii) renaturing the DNA to form double stranded DNA which can include sense/antisense pairs from different nicked products; (iv) removing single stranded portions from reformed duplexes by treatment with S1 nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of Delta3 homologs. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Each of the illustrative assays described below are amenable to high through-put analysis as necessary to screen large numbers of degenerate Delta3 sequences created by combinatorial mutagenesis techniques.

Combinatorial mutagenesis has a potential to generate very large libraries of mutant proteins, e.g., in the order of 1026 molecules. Combinatorial libraries of this size may be technically challenging to screen even with high throughput screening assays. To overcome this problem, a new technique has been developed recently, recrusive ensemble mutagenesis (REM), which allows one to avoid the very high proportion of non-functional proteins in a random library and simply enhances the frequency of functional proteins, thus decreasing the complexity required to achieve a useful sampling of sequence space. REM is an algorithm which enhances the frequency of functional mutants in a library when an appropriate selection or screening method is employed (Arkin and Yourvan, 1992, PNAS USA 89:7811-7815; Yourvan et al., 1992, Parallel Problem Solving from Nature, 2., In Maenner and Manderick, eds., Elsevir Publishing Co., Amsterdam, pp. 401-410; Delgrave et al., 1993, Protein Engineering 6(3):327-331).

The invention also provides for reduction of the Delta3 proteins to generate mimetics, e.g., peptide or non-peptide agents, which are able to bind to a Delta3 protein and/or to disrupt binding of a Delta3 polypeptide of the present invention with either upstream or downstream components of a Delta/Notch signaling cascade, such as binding proteins or interactors. Thus, such mutagenic techniques as described above are also useful to map the determinants of the Delta3 proteins which participate in protein-protein interactions involved in, for example, binding of the subject Delta3 polypeptide to proteins which may function upstream (including both activators and repressors of its activity) or to proteins or nucleic acids which may function downstream of the Delta3 polypeptide, whether they are positively or negatively regulated by it, for example, Notch. To illustrate, the critical residues of a subject Delta3 polypeptide which are involved in molecular recognition of, for example, the Notch gene product or other component upstream or downstream of a Delta3 gene can be determined and used to generate Delta-derived peptidomimetics which competitively inhibit binding of the authentic Delta3 protein with that moiety. By employing, for example, scanning mutagenesis to map the amino acid residues of each of the subject Delta3 proteins which are involved in binding other extracellular proteins, peptidomimetic compounds can be generated which mimic those residues of the Delta3 protein which facilitate the interaction. Such mimetics may then be used to interfere with the normal function of a Delta3 protein. For instance, non-hydrolyzable peptide analogs of such residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), b-turn dipeptide cores (Nagai et al. (1985) Tetrahedron Lett 26:647; and Sato et al. (1986) J Chem Soc Perkin Trans 1:1231), and b-aminoalcohols (Gordon et al. (1985) Biochem Biophys Res Commun 126:419; and Dann et al. (1986) Biochem Biophys Res Commun 134:71).

4.3.1. Cells Expressing Delta3 Polypeptides

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel; Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident ë prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316, incorporated herein in its entirety, and European Application Publication NO: 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells, such as CHO cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. When cells are transfected with a stable construct, the introduced nucleic acid can be selected for, and also identified by drug selection (e.g., cells that have incorporated the selectable marker gene will survive, while the other cells die).

In another embodiment, the expression characteristics of an endogenous (e.g., Delta3) nucleic acid within a cell, cell line or microorganism may be modified by inserting a DNA regulatory element heterologous to the endogenous gene of interest into the genome of a cell, stable cell line or cloned microorganism such that the inserted regulatory element is operatively linked with the endogenous gene (e.g., Delta3) and controls, modulates or activates the endogenous gene. For example, endogenous Delta3 which is normally “transcriptionally silent”, i.e., Delta3 which is normally not expressed, or is expressed only at very low levels in a cell line or microorganism, may be activated by inserting a regulatory element which is capable of promoting the expression of a normally expressed gene product in that cell line or microorganism. Alternatively, transcriptionally silent, endogenous Delta3 may be activated by insertion of a promiscuous regulatory element that works across cell types.

A heterologous regulatory element may be inserted into a stable cell line or cloned microorganism, such that it is operatively linked with and activates expression of endogenous Delta3, using techniques, such as targeted homologous recombination, which are well known to those of skill in the art, and described e.g., in Chappel, U.S. Pat. No. 5,272,071, incorporated herein by reference in its entirety; PCT publication NO: WO 91/06667, published May 16, 1991.

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191, each of which are incorporated herein by reference in their entirety, and in Hogan, Manipulating the Mouse Embryo, (Cold Spring Harbor Laboratory Press, Cold Spring Harbor, N.Y., 1986) and Wakayama et al., (1999), Proc. Natl. Acad. Sci. USA, 96:14984-14989. Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NoS. WO 90/11354 (Nov. 2, 1990), WO 91/01140 (May 28, 1991), WO 92/0968, and WO 93/04169 (Mar. 6, 1997).

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication Nos. WO 97/07668 (Mar. 6, 1997) and WO 97/07669 (Mar. 6, 1997).

In other embodiments transgenic animals, described in more detail below can be used to produce recombinant proteins.

4.3.2 Fusion Proteins and Immunogens

In another embodiment, the coding sequences for the polypeptide can be incorporated as a part of a fusion gene including a nucleotide sequence encoding a different polypeptide.

In one embodiment, the Delta3 polypeptide is a Delta3-Ig polypeptide. The Delta3-Ig polypeptide can comprise the entire extracellular domain of Delta3, e.g., human Delta3, or a variant thereof. For example, a Delta3-Ig polypeptide can comprise an amino acid sequences from about amino acid 1 to about amino acid 529 of SEQ ID NO: 2 or from about amino acid 1 to about amino acid 530 of SEQ ID NO: 25. Other preferred Delta3-Ig proteins do not comprise a signal peptide and thus, preferably do not comprise about amino acid 1 to about amino acid 17 or amino acid 18 of SEQ ID NO: 2 or 25. Alternatively, a Delta3-Ig fusion protein can comprise a portion of the extracellular domain of a Delta3 protein or a variant of a portion of the extracellular domain of a Delta3 protein. Preferred portions of the extracellular domain include portions having at least one motif amino terminal to the transmembrane domain shown in FIG. 2. For example a Delta3-Ig fusion protein can comprise at least one EGF-like domain. A Delta3-Ig fusion protein can further comprise a DSL domain. A Delta3-Ig fusion protein can also further comprise a signal peptide. Delta3-Ig fusion proteins can be prepared as described, e.g., in U.S. Pat. No. 5,434,131.

This type of expression system can be useful under conditions where it is desirable to produce an immunogenic fragment of a Delta3 protein. For example, the VP6 capsid protein of rotavirus can be used as an immunologic carrier protein for portions of the Delta3 polypeptide, either in the monomeric form or in the form of a viral particle. The nucleic acid sequences corresponding to the portion of a subject Delta3 protein to which antibodies are to be raised can be incorporated into a fusion gene construct which includes coding sequences for a late vaccinia virus structural protein to produce a set of recombinant viruses expressing fusion proteins comprising Delta3 epitopes as part of the virion. It has been demonstrated with the use of immunogenic fusion proteins utilizing the Hepatitis B surface antigen fusion proteins that recombinant Hepatitis B virions can be utilized in this role as well. Similarly, chimeric constructs coding for fusion proteins containing a portion of a Delta3 protein and the poliovirus capsid protein can be created to enhance immunogenicity of the set of polypeptide antigens (see, for example, EP Publication No: 0259149; and Evans et al. (1989) Nature 339:385; Huang et al. (1988) J. Virol. 62:3855; and Schlienger et al. (1992) J. Virol. 66:2).

The Multiple Antigen Peptide system for peptide-based immunization can also be utilized to generate an immunogen, wherein a desired portion of a Delta3 polypeptide is obtained directly from organo-chemical synthesis of the peptide onto an oligomeric branching lysine core (see, for example, Posnett et al. (1988) J. Biol. Chem. 263:1719 and Nardelli et al. (1992) J. Immunol. 148:914). Antigenic determinants of Delta3 proteins can also be expressed and presented by bacterial cells.

In addition to utilizing fusion proteins to enhance immunogenicity, it is widely appreciated that fusion proteins can also facilitate the expression of proteins, and accordingly, can be used in the expression of the Delta3 polypeptides of the present invention. For example, Delta3 polypeptides can be generated as glutathione-S-transferase (GST-fusion) proteins. Such GST-fusion proteins can enable easy purification of the Delta3 polypeptide, as for example by the use of glutathione-derivatized matrices (see, for example, Current Protocols in Molecular Biology, eds. Ausubel et al. (N.Y.: John Wiley & Sons, 1991)).

In another embodiment, a fusion gene coding for a purification leader sequence, such as a poly-(His)/enterokinase cleavage site sequence at the N-terminus of the desired portion of the recombinant protein, can allow purification of the expressed fusion protein by affinity chromatography using a Ni²⁺ metal resin. The purification leader sequence can then be subsequently removed by treatment with enterokinase to provide the purified protein (e.g., see Hochuli et al. (1987) J. Chromatography 411:177; and Janknecht et al. Proc. Natl. Acad. Sci. USA 88:8972). Techniques for making fusion genes are known to those skilled in the art. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al. John Wiley & Sons: 1992).

4.3.3. Antibodies

Another aspect of the invention pertains to an antibody that binds to a Delta3 protein; that is, to antibodies directed against a polypeptide of the invention. For example, by using immunogens derived from a Delta3 protein, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a Delta3 polypeptide or an antigenic fragment which is capable of eliciting an antibody response, or a fusion protein as described above). Techniques for conferring immunogenicity on a protein or peptide include conjugation to carriers or other techniques well known in the art. An immunogenic portion of a Delta3 protein can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of a Delta3 polypeptide, anti-Delta3 antisera can be obtained and, if desired, polyclonal anti-Delta3 antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a Delta3 polypeptide of the present invention and monoclonal antibodies isolated from a culture comprising such hybridoma cells. In one embodiment anti-human Delta3 antibodies specifically react with the proteins encoded by the DNA of ATCC® Deposit Accession Number 98348.

Antibodies can be fragmented using conventional techniques and the fragments screened for utility in the same manner as described above for whole antibodies. For example, F(ab)2 fragments can be generated by treating antibody with pepsin. The resulting F(ab)2 fragment can be treated to reduce disulfide bridges to produce Fab fragments. The antibody of the present invention is further intended to include bispecific and chimeric molecules having affinity for a Delta3 protein conferred by at least one CDR region of the antibody.

Antibodies which specifically bind Delta3 epitopes can also be used in immunohistochemical staining of tissue samples in order to evaluate the abundance and pattern of expression of each of the subject Delta3 polypeptides. Anti-Delta3 antibodies can be used diagnostically in immuno-precipitation and immuno-blotting to detect and evaluate Delta3 protein levels in tissue as part of a clinical testing procedure. For instance, such measurements can be useful in predictive valuations of the onset or progression of neurodegenerative, neoplastic or hyperplastic disorders. Likewise, the ability to monitor Delta3 protein levels in an individual can allow determination of the efficacy of a given treatment regimen for an individual afflicted with such a disorder. The level of Delta3 polypeptides may be measured from cells in bodily fluid, such as in samples of cerebral spinal fluid or amniotic fluid, or can be measured in tissue, such as produced by biopsy. Diagnostic assays using anti-Delta3 antibodies can include, for example, immunoassays designed to aid in early diagnosis of a neurodegenerative disorder, particularly ones which are manifest at birth. Diagnostic assays using anti-Delta3 polypeptide antibodies can also include immunoassays designed to aid in early diagnosis and phenotyping neurodegenerative, neoplastic or hyperplastic disorders.

Another application of anti-Delta3 antibodies of the present invention is in the immunological screening of cDNA libraries constructed in expression vectors such as λgt11, λgt18-23, λZAP, and ORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of a Delta3 protein, e.g., other orthologs of a particular Delta3 protein or other paralogs from the same species, can then be detected with antibodies, as, for example, reacting nitrocellulose filters lifted from infected plates with anti-Delta3 antibodies. Positive phage detected by this assay can then be isolated from the infected plate. Thus, the presence of Delta3 homologs can be detected and cloned from other animals, as can alternate isoforms (including splicing variants) from humans.

Polyclonal antibodies can be prepared as described above by immunizing a suitable subject with a polypeptide of the invention as an immunogen. Preferred polyclonal antibody compositions are ones that have been selected for antibodies directed against a polypeptide or polypeptides of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a non-human host cell for recombinant expression of a polypeptide of the invention. In such a manner, the only human epitope or epitopes recognized by the resulting antibody compositions raised against this immunogen will be present as part of a polypeptide or polypeptides of the invention.

The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. Alternatively, antibodies specific for a protein or polypeptide of the invention can be selected for (e.g., partially purified) or purified by, e.g., affinity chromatography. For example, a recombinantly expressed and purified (or partially purified) protein of the invention is produced as described herein, and covalently or non-covalently coupled to a solid support such as, for example, a chromatography column. The column can then be used to affinity purify antibodies specific for the proteins of the invention from a sample containing antibodies directed against a large number of different epitopes, thereby generating a substantially purified antibody composition, i.e., one that is substantially free of contaminating antibodies. By a substantially purified antibody composition is meant, in this context, that the antibody sample contains at most only 30% (by dry weight) of contaminating antibodies directed against epitopes other than those on the desired protein or polypeptide of the invention, and preferably at most 20%, yet more preferably at most 10%, and most preferably at most 5% (by dry weight) of the sample is contaminating antibodies. A purified antibody composition means that at least 99% of the antibodies in the composition are directed against the desired protein or polypeptide of the invention.

At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog NO: 27-9400-01; and the Stratagene SurfZAP Phage Display Kit, Catalog NO: 240612). Additionally, examples of methods and reagents particularly amenable for use in generating and screening antibody display library can be found in, for example, U.S. Pat. No. 5,223,409; PCT Publication No: WO 92/18619; PCT Publication No: WO 91/17271; PCT Publication No: WO 92/20791; PCT Publication No: WO 92/15679; PCT Publication No: WO 93/01288; PCT Publication No: WO 92/01047; PCT Publication No: WO 92/09690; PCT Publication No: WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816,397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089.) Such chimeric and humanized monoclonal antibodies can be produced by recombinant DNA techniques known in the art, for example using methods described in PCT Publication NO: WO 87/02671; European Patent Application 184,187; European Patent Application 171,496; European Patent Application 173,494; PCT Publication NO: WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Inmunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced, for example, using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope. (Jespers et al. (1994) Bio/technology 12:899-903).

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Further, an antibody (or fragment thereof) can be conjugated to a therapeutic moiety such as a cytotoxin, a therapeutic agent or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers such as, for example, lymphokines, interleukin-1(“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62:119-58 (1982).

Alternatively, an antibody can be conjugated to a second antibody to form an antibody heteroconjugate as described by Segal in U.S. Pat. No. 4,676,980, incorporated herein by reference in its entirety.

Accordingly, in one aspect, the invention provides substantially purified antibodies or fragment thereof, and human and non-human antibodies or fragments thereof, which antibodies or fragments specifically bind to a polypeptide comprising an amino acid sequence selected from the group consisting of: the amino acid sequence of any one of SEQ ID NOs: 2, 25, 28, 30, 32, 34, 36, 38, 40, 42, 44 or 46, or an amino acid sequence encoded by the cDNA of a clone deposited as ATCC® 98348; a fragment of at least 15 amino acid residues of the amino acid sequence of any one of SEQ ID NOs: 2, 25, 28, 30, 32, 34, 36, 38, 40, 42, 44 or 46, an amino acid sequence which is at least 95% identical to the amino acid sequence of any one of SEQ ID NOs: 2, 25, 28, 30, 32, 34, 36, 38, 40, 42, 44 or 46, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID Nos: 1, 3, 24, 26, 27, 29, 31, 33, 35, 37, 39, 41, 43 or 45, or the cDNA of a clone deposited as ATCC® 98348, or a complement thereof, under conditions of hybridization of 6× SSC at 45□C. and washing in 0.2× SSC, 0.1% SDS at 65□C. In various embodiments, the substantially purified antibodies of the invention, or fragments thereof, can be human, non-human, chimeric and/or humanized antibodies.

In another aspect, the invention provides human and non-human antibodies or fragments thereof, which antibodies or fragments specifically bind to a polypeptide comprising an amino acid sequence selected from the group consisting of: the amino acid sequence of any one of SEQ ID NOs: 2, 25, 28, 30, 32, 34, 36, 38, 40, 42, 44 or 46, or an amino acid sequence encoded by the cDNA of a clone deposited as ATCC® 98348; a fragment of at least 15 amino acid residues of the amino acid sequence of any one of SEQ ID NOs: 2, 25, 28, 30, 32, 34, 36, 38, 40, 42, 44 or 46, an amino acid sequence which is at least 95% identical to the amino acid sequence of any one of SEQ ID NOs: 2, 25, 28, 30, 32, 34, 36, 38, 40, 42, 44 or 46, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID Nos:1, 3, 24, 26, 27, 29, 31, 33, 35, 37, 39, 41, 43 or 45, or the cDNA of a clone deposited as ATCC® 98348, or a complement thereof, under conditions of hybridization of 6× SSC at 45□C. and washing in 0.2× SSC, 0.1% SDS at 65□C. Such non-human antibodies can be goat, mouse, sheep, horse, chicken, rabbit, or rat antibodies. Alternatively, the non-human antibodies of the invention can be chimeric and/or humanized antibodies. In addition, the non-human antibodies of the invention can be polyclonal antibodies or monoclonal antibodies.

In still a further aspect, the invention provides monoclonal antibodies or fragments thereof, which antibodies or fragments specifically bind to a polypeptide comprising an amino acid sequence selected from the group consisting of: the amino acid sequence of any one of SEQ ID NOs: 2, 25, 28, 30, 32, 34, 36, 38, 40, 42, 44 or 46, or an amino acid sequence encoded by the cDNA of a clone deposited as ATCC® 98348; a fragment of at least 15 amino acid residues of the amino acid sequence of any one of SEQ ID NOs: 2, 25, 28, 30, 32, 34, 36, 38, 40, 42, 44 or 46, an amino acid sequence which is at least 95% identical to the amino acid sequence of any one of SEQ ID NOs: 2, 25, 28, 30, 32, 34, 36, 38, 40, 42, 44 or 46, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID NOs: 1, 3, 24, 26, 27, 29, 31, 33, 35, 37, 39, 41, 43 or 45, or the cDNA of a clone deposited as any of ATCC® 98348, or a complement thereof, under conditions of hybridization of 6× SSC at 45□C. and washing in 0.2× SSC, 0.1% SDS at 65□C. The monoclonal antibodies can be human, humanized, chimeric and/or non-human antibodies.

The substantially purified antibodies or fragments thereof specifically bind to a signal peptide, a secreted sequence, an extracellular domain, a transmembrane or a cytoplasmic domain cytoplasmic membrane of a polypeptide of the invention. In a particularly preferred embodiment, the substantially purified antibodies or fragments thereof, the human and non-human antibodies or fragments thereof, and/or the monoclonal antibodies or fragments thereof, of the invention specifically bind to a secreted sequence or an extracellular domain of the amino acid sequence of SEQ ID NOs: 2, 25, 28, 30, 32, 34, 36, 38, 40, 42, 44 or 46. Preferably, the secreted sequence or extracellular domain to which the antibody, or fragment thereof, binds comprises from about amino acids 1-529 or 18-529 of SEQ ID NO: 2, or from amino acids 1-530 or 18-530 of SEQ ID NO: 25.

Any of the antibodies of the invention can be conjugated to a therapeutic moiety or to a detectable substance. Non-limiting examples of detectable substances that can be conjugated to the antibodies of the invention are an enzyme, a prosthetic group, a fluorescent material, a luminescent material, a bioluminescent material, and a radioactive material.

The invention also provides a kit containing an antibody of the invention, and instructions for use. In another embodiment, a kit comprising an antibody of the invention conjugated to a detectable substance and instructions for use. Still another aspect of the invention is a pharmaceutical composition comprising an antibody of the invention and a pharmaceutically acceptable carrier. In preferred embodiments, the pharmaceutical composition contains an antibody of the invention, a therapeutic moiety, and a pharmaceutically acceptable carrier.

Still another aspect of the invention is a method of making an antibody that binds, that is, is directed against, Delta3, the method comprising immunizing a mammal with a polypeptide. The polypeptide used as an immungen comprises an amino acid sequence selected from the group consisting of: the amino acid sequence of any one of SEQ ID NOs: 2, 25, 28, 30, 32, 34, 36, 38, 40, 42, 44 or 46, or an amino acid sequence encoded by the cDNA of a clone deposited as ATCC® 98348; a fragment of at least 15 amino acid residues of the amino acid sequence of any one of SEQ ID NOs: 2, 25, 28, 30, 32, 34, 36, 38, 40, 42, 44 or 46, an amino acid sequence which is at least 95% identical to the amino acid sequence of any one of SEQ ID NOs: 2, 25, 28, 30, 32, 34, 36, 38, 40, 42, 44 or 46, wherein the percent identity is determined using the ALIGN program of the GCG software package with a PAM120 weight residue table, a gap length penalty of 12, and a gap penalty of 4; and an amino acid sequence which is encoded by a nucleic acid molecule which hybridizes to the nucleic acid molecule consisting of any one of SEQ ID NOs:1, 3, 24, 26, 27, 29, 31, 33, 35, 37, 39, 41, 43 or 45, or the cDNA of a clone deposited as ATCC® 98348, or a complement thereof, under conditions of hybridization of 6× SSC at 45□C. and washing in 0.2× SSC, 0.1% SDS at 65□C. After immunization, a sample is collected from the mammal that contains an antibody that specifically recognizes Delta3. Preferably, the polypeptide is recombinantly produced using a non-human host cell. Optionally, the antibodies can be further purified from the sample using techniques well known to those of skill in the art. The method can further comprise producing a monoclonal antibody-producing cell from the cells of the mammal. Optionally, antibodies are collected from the antibody-producing cell.

4.4 Methods of Treating Disease

Based at least in part on the fact that the Notch signaling pathway has been implicated in development of the nervous system, in particular in regulating neuronal differentiation and vasculature, e.g., CNS vasculature, a wide variety of pathological diseases or conditions can benefit from treatment with Delta3 nucleic acids, proteins, and modulators thereof. In particular, based at least in part on the observation that PS1 and PS2, genes encoding amyloid precursor proteins, which are mutated in about 10% of cases of Alzheimer's disease, are functionally linked to the Notch signaling pathway, mutations in genes of the Notch signaling pathway, e.g., Delta genes, could also result in Alzheimer's disease or other neurodegenerative or neuro-developmental diseases. The Notch signaling pathway plays a role in the development of vasculature. For example, loss of D111 function mutants become severally hemorrhagic after embryonic day 10. Furthermore, mutations in Notch3 result in CADASIL, a disease characterized by stroke. In addition, mice with a functionally ablated PS1 gene exhibit hemorrhages in the brain and/or spinal cord after embryonic day 11.5 (Wong et al. (1997) Nature 387:288). In addition, the Notch pathway has been implicated in hematologic development Specifically, molecules in the Notch signaling pathway have been shown to be expressed in a wide variety of blood cells, including but not limited to those of myeloid and lymphoid origins. Notch-1 was shown to play a role in T-cell development. Furthermore, since the Notch signaling pathway is involved in cell fate determination at least in the nervous system, immune system and endothelial system, it is likely that the Notch signaling pathway, and in particular Delta3 is involved in cell fate determination in additional biological systems. Accordingly, the invention also provides methods for treating diseases or disorders arising from an abnormal cell proliferation and/or differentiation of cells other than cells from the nervous system, immune system, and vasculature.

Among the disorders that can be treated or prevented according to the methods of the invention include pathological neurogenic, neoplastic or hyperplastic conditions. Neurologic diseases, e.g., neurodegenerative, neuro-differentiative and neuro-developmental diseases, that might benefit from this methodology include, but are not limited to neuropathies, e.g., peripheral neuropathy such as ACCPN, stroke, dementia, e.g., cerebral autosomal dominant arteriopathy with subcortical infarcts and leukoencephalopathy (CADASIL), degenerative lesions (Parkinson's disease, Alzheimer's disease, Huntington's chorea, amyotrophic lateral sclerosis, spinocerebellar degenerations), demyelating diseases (multiple sclerosis, human immunodeficiency associated myelopathy, transverse myelopathy, progressive multifocal leukoencephalopathy, pontine myelinolysis), motor neuron injuries, progressive spinal muscular atrophy, progressive bulbar palsy, primary lateral sclerosis, infantile and juvenile muscular atrophy, progressive bulbar paralysis of childhood (Fazio-Londe syndrome), poliomyelitis, and hereditary motorsensory neuropathy (Charcot-Marie-Tooth disease), spinal cord injuries, brain injuries, lesions associated with surgery, ischemic lesions, malignant lesions, infectious lesions.

Additional neurological diseases that can be treated according to the method of the invention include neuropathies, e.g., peripheral neuropathies, e.g., Agenesis of the Corpus Callosum with Peripheral Neuropathy (ACCPN). In fact, as set forth in the examples presented below, hDelta3 has been mapped to human chromosome 15 close to framework markers D15S1244 and D15S144, a chromosomal region which has been shown to be genetically linked (ACCPN) (Casaubon et al. (1996) Am J. Hum. Genet. 58:28). The disease is characterized by a progressive peripheral neuropathy caused by axonal degeneration and a central nervous system (CNS) malformation characterized by the absence of hypoplasia of the corpus callosum. The disorder appears early in life, is progressive and results in death in the third decade of life of the subject.

Neuropathies refer to disorders of peripheral nerves and includes both motor and sensory functions, since most motor and sensory axons run in the same nerves. Neurophathies may be either chronic or acute. One example of a acute neuropathy is the Guillain-Barre syndrome, which often follow respiratory infection. Chronic neuropathies include, e.g., acute intermittent porphyria, Charcot-Marie-Tooth disease, metabolic diseases such as diabetes, obesity, and B12 deficiency, intoxication, nutritional disorders.

Disorders of the vasculature, also termed “vascular disorders”, in addition to CADASIL and stroke, that can be treated or prevented according to the methods of the invention include atheroma, tumor angiogenesis, wound healing, diabetic retinopathy, hemangioma, psoriasis, and restenosis, e.g., restenosis resulting from balloon angioplasty.

In one embodiment, diseases or disorders caused or contributed to by aberrant Delta3 activity, such as aberrant Delta3 protein levels or an aberrant biological activity or which are associated with one or more specific Delta3 alleles, e.g., a mutant Delta3 allele, can be treated with Delta3 therapeutics. Aberrant protein levels can be caused, e.g., by aberrant gene expression. Such aberrant activity can result, for example, in aberrant cell proliferation and/or differentiation or cell death. For example, aberrant Delta3 activity in a subject can result in increased proliferation of certain cells in the subject. Subjects having a disorder characterized by abnormal cell proliferation can be treated by administration of a Delta3 therapeutic inhibiting or decreasing such proliferation. The specific Delta3 therapeutic used may vary depending on the type of the cell that is proliferating aberrantly. The appropriate Delta3 therapeutic to use can be determined, e.g., by in vitro culture of a sample of such cells which can be obtained from the subject, in the presence and in the absence of Delta3 therapeutics.

Diseases or conditions associated with aberrant cell proliferation which can be treated or prevented with Delta3 therapeutics include cancers, malignant conditions, premalignant conditions, benign conditions. The condition to be treated or prevented can be a solid tumor, such as a tumor arising in an epithelial tissue. For example, the cancer can be colon or cervix cancer. Cancer of the colon and cervix have in fact been found to have increased levels of expression of Notch as compared to normal tissue (PCT Publication No. WO/07474, Apr. 14, 1994). Accordingly, treatment of such a cancer could comprise administration to the subject of a Delta3 therapeutic decreasing the interaction of Notch with Delta3. Other cancers that can be treated or prevented with a Delta3 protein include sarcomas and carcinomas, e.g., lung cancer, cancer of the esophagus, lung cancer, melanoma, seminoma, and squamous adenocarcinoma. Additional solid tumors within the scope of the invention include those that can be found in a medical textbook. The condition to be treated or prevented can also be a soluble tumor, such as leukemia, either chronic or acute, including chronic or acute myelogenous leukemia, chronic or acute lymphocytic leukemia, promyelocytic leukemia, monocytic leukemia, myelomonocytic leukemia, and erythroleukemia. Yet other proliferative disorders that can be treated with a Delta3 therapeutic of the invention include heavy chain disease, multiple myeloma, lymphoma, e.g., Hodgkin's lymphoma and non-Hodgkin's lymphoma, Waldenstroem's macroglobulemia, and fibroproliferative disorders, particularly of cerebravascular tissue.

Diseases or conditions characterized by a solid or soluble tumor can be treated by administrating a Delta3 therapeutic either locally or systemically, such that proliferation of the cells having an aberrant proliferation is inhibited or decreased. Methods for administering the compounds of the invention are further described below.

The invention also provides methods for preventing the formation and/or development of tumors. For example, the development of a tumor can be preceded by the presence of a specific lesion, such as a pre-neoplastic lesion, e.g., hyperplasia, metaplasia, and dysplasia. Such lesions can be found, e.g., in epithelial tissue. Thus, the invention provides a method for inhibiting progression of such a lesion into a neoplastic lesion, comprising administering to the subject having a preneoplastic lesion a amount of a Delta3 therapeutic sufficient to inhibit progression of the preneoplastic lesion into a neoplastic lesion.

In a preferred embodiment, the invention provides a method for inhibiting endothelial cell proliferation and/or differentiation, comprising contacting a Delta3 therapeutic with a tissue in which endothelial cells are proliferating, such as a developing tumor or a hyperproliferative disease, i.e., a disease associated with abnormal cellular proliferation. Blocking the proliferation of endothelial cells will result in inhibition of development of endothelium and blood vessels, thus limiting access to the tumor of compounds necessary for tumor development.

The invention also provides for methods for treating or preventing diseases or conditions associated with insufficient cell proliferation. For example, Delta3 therapeutics can be used to stimulate tissue repair, regeneration, and/or wound healing, e.g., of neural tissue, such as after surgery or to stimulate tissue healing from burns. Other disease in which proliferation of cells is desired are hypoproliferative diseases, i.e., diseases characterized by an abnormally low proliferation of certain cells.

In yet another embodiment, the invention provides a method for treating or preventing diseases or conditions characterized by aberrant cell differentiation. Accordingly, the invention provides methods for stimulating cellular differentiation in conditions characterized by an inhibition of normal cell differentiation which may or may not be accompanied by excessive proliferation. Alternatively, Delta3 therapeutics can be used to inhibit differentiation of specific cells.

In one method, the aberrantly proliferating and/or differentiating cell is a cell present in the nervous system. Accordingly, the invention provides methods for treating diseases or conditions associated with a central or peripheral nervous system. For example, the invention provides methods for treating lesions of the nervous system involving an aberrant Delta3 activity in neurons, in Schwann cells, glial cells, or other types of neural cells. Disorders of the nervous system are set forth above.

In another embodiment, a Delta3 therapeutic can be utilized to ameliorate a symptom of obesity and/or disorders that accompany or are exacerbated by an obese state, such as cardiovascular and circulatory disorders, metabolic abnormalities typical of obesity, such as hyperinsulinemia, insulin resistance, diabetes, including non-insulin dependent diabetes mellitus (NIDDM), insulin dependent diabetes mellitus (IDDM), and maturity onset diabetes of the young (MODY), disorders of energy homeostasis, disorders associated with lipid metabolism, such as cachexia.

With respect to cardiovascular disorders, symptoms of coronary diseases (e.g., cardiovascular diseases including unstable angina pectoris, myocardial infarction, acute myocardial infarction, coronary artery disease, coronary revascularization, coronary restenosis, ventricular thromboembolism, atherosclerosis, coronary artery disease (e.g., arterial occlusive disorders), plaque formation, cardiac ischemia, including complications related to coronary procedures, such as percutaneous coronary artery angioplasty (balloon angioplasty) procedures) can be ameliorated. With respect to coronary procedures, such modulation can be achieved via administration of Delta3 therapeutics prior to, during, or subsequent to the procedure.

Delta3 therapeutics (e.g., nucleic acids, proteins and modulators thereof) can, therefore, be used to modulate disorders resulting from any blood vessel insult that can result in platelet aggregation. Such blood vessel insults include, but are not limited to, vessel wall injury, such as vessel injuries that result in a highly thrombogenic surface exposed within an otherwise intact blood vessel e.g., vessel wall injuries that result in release of ADP, thrombin and/or epinephrine, fluid shear stress that occurs at the site of vessel narrowing, ruptures and/or tears at the sites of atherosclerotic plaques, and injury resulting from balloon angioplasty or atherectomy. Preferably, such therapeutics do not effect initial platelet adhesion to vessel surfaces, or effect such adhesion to a relatively lesser extent than the effect on platelet-platelet aggregation, e.g., unregulated platelet-platelet aggregation, following the initial platelet adhesion.

In addition, Delta3 therapeutics can be utilized to amieliorate a symptom of disorders associated with abnormal vasculogenesis (e.g., cancers, including, but not limited to, cancers of the epithelia (e.g., carcinomas of the pancreas, stomach, liver, secretory glands (e.g., adenocarcinoma), bladder, lung, breast, skin (e.g., fibromatosis or malignant melanoma), reproductive tract including prostate gland, ovary, cervix and uterus); cancers of the hematopoietic and immune system (e.g., leukemias and lymphomas); cancers of the central nervous, brain system and eye (e.g., gliomas, neuroblastoma and retinoblastoma); and cancers of connective tissues, bone, muscles and vasculature (e.g., hemangiomas and sarcomas)), disorders related to fetal development, in particular, disorders involving development of lung and kidney, lung-related disorders, and immune-related disorders, such as inflammatory-related disorders, e.g., asthma, allergy, and autoimmune disorders, as well as neurological disorders, including developmental, cognitive and personality-related disorders, renal disorders, adrenal gland-related disorders; and disorders relating to skeletal muscle, such as dystrophic disorders.

With respect to immune disorders, Delta3 therapeutics (e.g., nucleic acids, proteins and modulators thereof) can be utilized to modulate processes involved in lymphocyte development, differentiation and activity, including, but not limited to development, differentiation and activation of T cells, including T helper, T cytotoxic and non-specific T killer cell types and subtypes, and B cells, immune functions associated with such cells, and amelioration of one or more symptoms associated with abnormal function of such cell types. Such disorders can include, but are not limited to, autoimmune disorders, such as organ specific autoimmune disorders, e.g., autoimmune thyroiditis, Type I diabetes mellitus, insulin-resistant diabetes, autoimmune anemia, multiple sclerosis, and/or systemic autoimmune disorders, e.g., rheumatoid arthritis, lupus or sclerodoma, allergy, including allergic rhinitis and food allergies, asthma, psoriasis, graft rejection, transplantation rejection, graft versus host disease, pathogenic susceptibilities, e.g., susceptibility to certain bacterial or viral pathogens, wound healing and inflammatory reactions.

With respect to skeletal muscle-related disorders, Delta3 therapeutics can be utilized to ameliorate symptoms of disorders including, for example, muscular dystrophy disorders, e.g., Duchenne's muscular dystrophy and X-linked recessive Emery-Dreifuss dystrophy (EDMD), as well as developmental and other disorders that involve skeletal muscle such as, for example, oculofacial-skeletal myorhythmias, sarcoidosis, and malignant hyperthermia susceptibility (MHS).

With respect to lung disorders, Delta3 therapeutics can be utilized, for exampe, to ameliorate a symptom of such pulmonary disorders, such as atelectasis, pulmonary congestion or edema, chronic obstructive airway disease (e.g., emphysema, chronic bronchitis, bronchial asthma, and bronchiectasis), diffuse interstitial diseases (e.g., sarcoidosis, pneumoconiosis, hypersensitivity pneumonitis, Goodpasture's syndrome, idiopathic pulmonary hemosiderosis, pulmonary alveolar proteinosis, desquamative interstitial pneumonitis, chronic interstitial pneumonia, fibrosing alveolitis, hamman-rich syndrome, pulmonary eosinophilia, diffuse interstitial fibrosis, Wegener's granulomatosis, lymphomatoid granulomatosis, and lipid pneumonia), or tumors (e.g., bronchogenic carcinoma, bronchiolovlveolar carcinoma, bronchial carcinoid, hamartoma, and mesenchymal tumors).

In another embodiment, the invention provides a method for enhancing the survival and/or stimulating proliferation and/or differentiation of cells and tissues in vitro. For example, tissues from a subject can be obtained and grown in vitro in the presence of a Delta3 therapeutic, such that the tissue cells are stimulated to proliferate and/or differentiate. The tissue can then be readministered to the subject.

In another embodiment, as Notch can function to maintain cells in an immature state in vitro, i.e., as stem cells, e.g., the invention provides a method to expand the pool of hematopoietic stem cells through the interaction of Delta3 with Notch, which can be utilized in cases where it is desirable to do so including, but not limited to preparing cells harvested for subsequent bone marrow transplantation. Thus, Delta 3 can be utilized in stem cell preservation, that is, can be utilized to preserve stem cells in an immature, undifferentiated state, and/or preserving the stem cells' pluripotency, differentiation potential and proliferation potential. In one embodiment of such a stem cell preservation method, stem cells are contacted with cells expressing Delta3 and exhibiting Delta3 on their cell surfaces. Such Delta3-expressing cells can be presented, e.g., as stromal cells in culture. In another embodiment, stem cells are contacted with full-length or soluble Delta3 attached to a solid surface, e.g., a culture plate, or microbeads.

In another embodiment, techniques such as that described above for stem cell preservation can be utilized to prevent death of CD4⁺/CD8⁺ T cells. Thus, such techniques can be used to repopulate peripheral T cell populations (e.g., as part of a leukemia therapy), or, and alternatively, can be used to produce and screen for an antigen-specific T cell clone.

In another embodiment, the invention can function as a method to determine the fate of T cells in the developing thymus. Antagonists or antagonists of hDelta3 activity can determine whether a T cell will develop the CD4 or CD8 phenotype and thus be useful as a therapeutic agent in immunodeficiency disorders, such as, but not limited to AIDS.

In another embodiment, as the Notch signaling pathway has been shown to be involved in eye development in Drosophila, and given the fact that mDelta3 was highly expressed in the developing mouse eye, Delta3 nucleic acids, proteins, and modulators thereof and/or agonists and antagonists can be used as therapeutics in such eye disorders as diabetic retinopathy, characterized by a hyper-proliferation of capillaries in the retina.

Since, in some cases, genes may be upregulated in a disease state and in other cases may be down-regulated, it will be desirable to activate and/or potentiate or suppress and/or down-modulate Delta3 activity depending on the condition to be treated using the techniques compounds and methods described herein. Some genes may be under-expressed in certain disease states. The activity of Delta3 nucleic acids, proteins, and modulators thereof may be in some way impaired, leading to the development of neurodegenerative disease symptoms. Such down-regulation of Delta3 gene expression or decrease in the activity of a Delta3 protein may have a causative or exacerbating effect on the disease state.

Among the approaches which may be used to ameliorate disease symptoms involving the misexpression of a Delta3 gene are, for example, antisense, ribozyme, and triple helix molecules described above. Compounds that compete with Delta3 nucleic acids, proteins, and modulators thereof for binding to upstream or downstream elements in a Delta/Notch signaling cascade will antagonize Delta3 nucleic acids, proteins, and modulators thereof, thereby inducing a therapeutic effect. Examples of suitable compounds include the antagonists or homologs described in detail above. In other instances, the increased expression or activity of Delta3 nucleic acids, proteins, and modulators thereof may be desirable and may be accomplished by, for example the use of Delta3 agonists or mimetics or by gene replacement therapy, as described herein.

Yet other Delta3 therapeutics comprise of a first peptide comprising a Delta3 peptide capable of binding to a receptor, e.g., a Notch receptor, and a second peptide which is cytotoxic. Such therapeutics can be used to specifically target and lyse cells expressing or over-expressing a receptor for Delta3. For example, a fusion protein containing a Delta3 peptide fused to a cytotoxic peptide can be used to eliminate or reduce a tumor over-expressing Notch, e.g., colon and cervix neoplastic tumors. Alternatively, cells expressing or over-expressing Delta3 can be targeted for lysis, by, for example, targeting to the cell an antibody binding specifically to a Delta3 protein linked to a cytotoxic peptide.

Based at least in part on the similarity of protein structure, it is likely that Delta3 nucleic acids, proteins, and modulators thereof can also be used to treat diseases or conditions caused by or contributed by an aberrant activity of a Delta family gene product, e.g., an aberrant Delta1 or Delta2 activity or diseases or disorders which are associated with one or more specific Delta alleles, e.g., Delta1 or Delta2 alleles. Such diseases or conditions could include neurological diseases and cancer. Similarly, Delta therapeutics, e.g., Delta1 or Delta2 therapeutics, could be used to prevent or treat diseases or disorders caused by or contributed to by an aberrant Delta3 activity, or diseases or disorders which are associated with a specific Delta3 allele. Delta therapeutics can be prepared using, e.g., the nucleotide and protein sequence information disclosed in the PCT Patent Publication WO 97/01571 and tested using the assays described herein for testing Delta3 therapeutics.

Compounds identified as increasing or decreasing Delta3 gene expression or protein activity can be administered to a subject at therapeutically effective dose to treat or ameliorate cardiovascular disease. A therapeutically effective dose refers to that amount of the compound sufficient to result in amelioration of symptoms associated with the particular disease.

4.4.1. Dosage and Formulation

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, intramuscular and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

In clinical settings, the gene delivery systems for the therapeutic Delta3 gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) Proc. Natl. Aacd. Sci. USA 91: 3054-3057). A Delta3 gene, such as any one of the sequences represented in the group consisting of SEQ ID NOs: 1, 3, 24, 26, 27, 29, 31, 33, 35, 37, 39, 41, 43 or 45, or a sequence homologous thereto can be delivered in a gene therapy construct by electroporation using techniques described, for example, by Dev et al. ((1994) Cancer Treat. Rev. 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

4.5 Diagnostic and Prognostic Assays

The present methods provides means for determining if a subject is at risk of developing a disorder characterized by an aberrant Delta3 activity, such as aberrant cell proliferation, degeneration, and/or differentiation resulting for example in a neurodegenerative disease or cancer. The invention also provides methods for determining whether a subject is at risk of developing a disease or disorder associated with one or more specific alleles of a Delta3 gene. In fact, specific Delta3 alleles may be associated with specific diseases or disorders. For example, at least one allele of hDelta3 is likely to be associated with the neurological disease ACCPN. Accordingly, the invention provides methods for determining whether a subject has or is at risk of developing a neurological disease, e.g., ACCPN. In another embodiment, the invention provides methods for determining whether a subject has or is at risk of developing a vascular disorder or a disorder associated with cell fate determination. In one embodiment, the invention comprises determining the identity of the Delta3 allele in a subject and comparing the molecular structure of the Delta3 gene of the subject with the molecular structure of a Delta3 gene from a subject which does not have the neurological disease. Determining the molecular structure can be, e.g., determining the identity of at least one nucleotide, determining the nucleotide composition or determining the methylation pattern of the gene.

In one embodiment, the invention provides a method for determining whether a subject has genetic lesion in a Delta3 gene or a specific allelic variant of a polymorphic region in a Delta3 gene. The specific allele can be a mutant allele. In another embodiment, the invention provides methods for determining whether a subject has an aberrant Delta3 protein, resulting from aberrant post-translational modifications of the protein, such as aberrant phosphorogulation or glycosylation. Also, within the scope of the invention are methods for determining whether a subject has an aberrant expression level of a Delta3 protein, which could be due to a genetic lesion in the Delta3 gene or due to an aberrant level or activity of a protein regulating the expression of a Delta3 gene.

In preferred embodiments, the methods can be characterized as comprising detecting, in a sample of cells from the subject, the presence or absence of a genetic lesion characterized by at least one of (i) an alteration affecting the integrity of a gene encoding a Delta-protein, or (ii) the mis-expression of a Delta3 gene. To illustrate, such genetic lesions can be detected by ascertaining the existence of at least one of (i) a deletion of one or more nucleotides from a Delta3 gene, (ii) an addition of one or more nucleotides to a Delta3 gene, (iii) a substitution of one or more nucleotides of a Delta3 gene, (iv) a gross chromosomal rearrangement of a Delta3 gene, (v) a gross alteration in the level of a messenger RNA transcript of a Delta3 gene, (vii) aberrant modification of a Delta3 gene, such as of the methylation pattern of the genomic DNA, (vii) the presence of a non-wild type splicing pattern of a messenger RNA transcript of a Delta3 gene, (viii) a non-wild type level of a Delta-protein, (ix) allelic loss of a Delta3 gene, and (x) inappropriate post-translational modification of a Delta-protein. As set out below, the present invention provides a large number of assay techniques for detecting lesions in a Delta3 gene, and importantly, provides the ability to discern between different molecular causes underlying Delta-dependent aberrant cell proliferation and/or differentiation.

For determining whether a subject has or is at risk of developing a disease or condition associated with a specific allele of a Delta3 gene, preliminary experiments can be performed to determine the identity of the allele associated with a disease. For example, for determining the identity of the hDelta3 allele associated with ACCPN, one can perform mutation detection studies of the Delta3 gene in populations having a high risk of developing ACCPN. For example, one can perform mutation detection analysis of the genomic DNA from subjects in the French Canadian population in the Charlevoix and Saguenay-Lac St Jean regions of the province of Quebec (Casaubon et al. (1996) Am. J. Hum. Genet. 58:28). Such an analysis will reveal the identity of the Delta3 allele or alleles associated with ACCPN. Comparison of the Delta3 allele of a subject with this allele or alleles associated with ACCPN will indicate whether a subject has a Delta3 allele associated with ACCPN and thus whether the subject has or is likely to develop ACCPN. Similarly, mutation detection analysis can also be carried out to determine the identity of Delta3 alles associated with other diseases or conditions.

In an exemplary embodiment, there is provided a nucleic acid composition comprising a (purified) oligonucleotide probe including a region of nucleotide sequence which is capable of hybridizing to a sense or antisense sequence of a Delta3 gene, such as represented by any of SEQ ID NOs: 1, 3, 24, 26, 27, 29, 31, 33, 35, 37, 39, 41, 43 or 45, alleles thereof, naturally-occurring mutants thereof, or 5′ or 3′ flanking sequences or intronic sequences naturally associated with the subject Delta3 genes or naturally-occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels.

As set out above, one aspect of the present invention relates to diagnostic assays for determining, in the context of cells isolated from a patient, if mutations have arisen in one or more Delta3 genes of the sample cells. The present method provides a method for determining if a subject is at risk for a disorder characterized by aberrant Delta3 activity, e.g., cell proliferation and/or differentiation. In preferred embodiments, the method can be generally characterized as comprising detecting, in a sample of cells from the subject, the presence or absence of a genetic lesion characterized by an alteration affecting the integrity of a gene encoding a Delta protein. To illustrate, such genetic lesions can be detected by ascertaining the existence of at least one of (i) a deletion of one or more nucleotides from a Delta-gene, (ii) an addition of one or more nucleotides to a Delta-gene, (iii) a substitution of one or more nucleotides of a Delta-gene, and (iv) the presence of a non-wild type splicing pattern of a messenger RNA transcript of a Delta-gene. As set out below, the present invention provides a large number of assay techniques for detecting lesions in Delta3 genes, and importantly, provides the ability to discern between different molecular causes underlying Delta-dependent aberrant cell proliferation and/or differentiation.

In certain embodiments, detection of the lesion in a Delta gene or the identity of an allelic variant of a polymorphic region of a Delta gene comprises utilizing the probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the Delta-gene (see Abravaya et al. (1995) Nuc Acid Res 23:675-682). In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize to a Delta gene under conditions such that hybridization and amplification of the Delta-gene (if present) occurs, and (iv) detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In a preferred embodiment of the subject assay, mutations in a Delta3 gene or specific alleles of a Delta3 gene from a sample cell are identified by alterations in restriction enzyme cleavage patterns. For example, sample and control DNA is isolated, amplified (optionally), digested with one or more restriction endonucleases, and fragment length sizes are determined by gel electrophoresis. Moreover, the use of sequence specific ribozymes (see, for example, U.S. Pat. No. 5,498,531, incorporated herein by reference in its entirety) can be used to score for the presence of specific mutations by development or loss of a ribozyme cleavage site.

In yet another embodiment, any of a variety of sequencing reactions known in the art can be used to directly sequence the Delta3 gene and detect mutations or allelic variants of polymorphic regions by comparing the sequence of the sample Delta3 with the corresponding wild-type (control) sequence. Exemplary sequencing reactions include those based on techniques developed by Maxim and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including by sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-tract or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA heteroduplexes (Myers, et al. (1985) Science 230:1242). In general, the art technique of “mismatch cleavage” starts by providing heteroduplexes of formed by hybridizing (labeled) RNA or DNA containing the wild-type Delta3 sequence with potentially mutant RNA or DNA obtained from a tissue sample. The double-stranded duplexes are treated with an agent which cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl Acad Sci USA 85:4397; Saleeba et al (1992) Methods Enzymod. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in Delta3 cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a Delta3 sequence, e.g., a wild-type Delta3 sequence, is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039, incorporated herein by reference in its entirety.

In other embodiments, alterations in electrophoretic mobility will be used to identify mutations in Delta3 genes or for determining the identity of the Delta3 allele. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control Delta3 nucleic acids will be denatured and allowed to renature. The secondary structure of single-stranded nucleic acids varies according to sequence, the resulting alteration in electrophoretic mobility enables the detection of even a single base change. The DNA fragments may be labelled or detected with labelled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet 7:5).

In yet another embodiment the movement of mutant or wild-type fragments in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al (1989) Proc. Natl Acad. Sci USA 86:6230). Such allele specific oligonucleotide hybridization techniques may be used to test one mutation per reaction when oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci USA 88:189). In such cases, ligation will occur only if there is a perfect match at the 3′ end of the 5′ sequence making it possible to detect the presence of a known mutation at a specific site by looking for the presence or absence of amplification.

Another embodiment of the invention provides for a nucleic acid composition comprising a (purified) oligonucleotide probe including a region of nucleotide sequence which is capable of hybridizing to a sense or antisense sequence of a Delta-gene, or naturally-occurring mutants thereof, or 5′ or 3′ flanking sequences or intronic sequences naturally associated with the subject Delta-genes or naturally-occurring mutants thereof. The nucleic acid of a cell is rendered accessible for hybridization, the probe is exposed to nucleic acid of the sample, and the hybridization of the probe to the sample nucleic acid is detected. Such techniques can be used to detect lesions at either the genomic or mRNA level, including deletions, substitutions, etc., as well as to determine mRNA transcript levels. Such oligonucleotide probes can be used for both predictive and therapeutic evaluation of allelic mutations which might be manifest in, for example, a neurodegenerative, neoplastic or hyperplastic disorders (e.g., aberrant cell growth).

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a Delta3 gene.

Any cell type or tissue, preferably neural or endothelial cells, in which the Delta3 is expressed may be utilized in the diagnostics described below. For example, a subject's bodily fluid (e.g., blood) can be obtained by known techniques (e.g., venipuncture). Alternatively, nucleic acid tests can be performed on dry samples (e.g., hair or skin). Fetal nucleic acid samples can be obtained from maternal blood as described in International Patent Application NO: WO91/07660 to Bianchi. Alternatively, amniocytes or chorionic villi may be obtained for performing prenatal testing, e.g., of ACCPN, which is a disease which is usually fatal in the third decade of life.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

Antibodies directed against wild type or mutant Delta3 proteins, which are discussed, above, may also be used in disease diagnostics and prognostics. Such diagnostic methods, may be used to detect abnormalities in the level of Delta3 protein expression, or abnormalities in the structure and/or tissue, cellular, or subcellular location of Delta3 proteins. Structural differences may include, for example, differences in the size, electronegativity, or antigenicity of the mutant Delta3 protein relative to the normal Delta3 protein. Protein from the tissue or cell type to be analyzed may easily be detected or isolated using techniques which are well known to one of skill in the art, including but not limited to western blot analysis. For a detailed explanation of methods for carrying out western blot analysis, see Sambrook et al, 1989, supra, at Chapter 18. The protein detection and isolation methods employed herein may also be such as those described in Harlow and Lane, for example, (Harlow, E. and Lane, D., 1988, “Antibodies: A Laboratory Manual”, Cold Spring Harbor Laboratory Press, Cold Spring Harbor, N.Y.).

This can be accomplished, for example, by immunofluorescence techniques employing a fluorescently labeled antibody (see below) coupled with light microscopic, flow cytometric, or fluorimetric detection. The antibodies (or fragments thereof) useful in the present invention may, additionally, be employed histologically, as in immunofluorescence or immunoelectron microscopy, for in situ detection of Delta3 proteins. In situ detection may be accomplished by removing a histological specimen from a patient, and applying thereto a labeled antibody of the present invention. The antibody (or fragment) is preferably applied by overlaying the labeled antibody (or fragment) onto a biological sample. Through the use of such a procedure, it is possible to determine not only the presence of the Delta3 protein, but also its distribution in the examined tissue. Using the present invention, one of ordinary skill will readily perceive that any of a wide variety of histological methods (such as staining procedures) can be modified in order to achieve such in situ detection.

Often a solid phase support or carrier is used as a support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

One means for labeling an anti-Delta3 protein specific antibody is via linkage to an enzyme and use in an enzyme immunoassay (EIA) (Voller, “The Enzyme Linked Immunosorbent Assay (ELISA)”, Diagnostic Horizons 2:1-7, 1978, Microbiological Associates Quarterly Publication, Walkersville, Md.; Voller, et al., J. Clin. Pathol. 31:507-520 (1978); Butler, Meth. Enzymol. 73:482-523 (1981); Maggio, (ed.) Enzyme Immunoassay, CRC Press, Boca Raton, Fla., 1980; Ishikawa, et al., (eds.) Enzyme Immunoassay, Kgaku Shoin, Tokyo, 1981). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may also be accomplished using any of a variety of other immunoassays. For example, by radioactively labeling the antibodies or antibody fragments, it is possible to detect fingerprint gene wild type or mutant peptides through the use of a radioimmunoassay (RIA) (see, for example, Weintraub, B., Principles of Radioimmunoassays, Seventh Training Course on Radioligand Assay Techniques, The Endocrine Society, March, 1986). The radioactive isotope can be detected by such means as the use of a gamma counter or a scintillation counter or by autoradiography.

It is also possible to label the antibody with a fluorescent compound. When the fluorescently labeled antibody is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as 152Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Moreover, it will be understood that any of the above methods for detecting alterations in a Delta3 gene or gene product can be used to monitor the course of treatment or therapy.

4.6. Drug Screening Assays

The invention provides for compounds, e.g., therapeutic compounds, for treating diseases or conditions caused by, or contributed to by an abnormal Delta3 activity. The compounds that can be used for this purpose can be any type of compound, including a protein, a peptide, peptidomimetic, small molecule, and nucleic acid. A nucleic acid can be, e.g., a gene, an antisense nucleic acid, a ribozyme, or a triplex molecule. A compound of the invention can be an agonist or an antagonist. A compound can act on a Delta3 gene, e.g., to modulate its expression. A compound can also act on a Delta3 protein, e.g., to modulate signal transduction from the receptor. Accordingly, a compound of the invention can be a compound which binds to Delta3 and induces signal transduction from the receptor, such that, e.g., a Delta3 activity is induced. Alternatively, a compound of the invention can be a compound which inhibits interaction of a Delta3 protein with a toporythmic protein, e.g., Notch. In one embodiment, a compound of the invention which interacts with a Delta protein, which is either an agonist or an antagonist, is a toporythmic protein or other protein interacting with Delta3. In an even more preferred embodiment, the compound is a soluble toporythmic protein or other protein interacting with Delta3. For example, a soluble antagonistic toporythmic protein can be a protein which competes with the wild type toporythmic proteins for binding to Delta3. A soluble agonistic toporythmic protein can be a protein which binds to a Delta3 protein in essentially the same manner as a wild-type toporythmic protein, such as to induce at least one Delta3 activity, e.g., signal transduction from the Delta3 protein. Accordingly, a soluble toporythmic protein can be stimulatory form of a toporythmic protein or an inhibitory form of a toporythmic, depending on whether the particular toporythmic protein stimulates or inhibits a Delta3 activity.

Similarly, a soluble Delta3 protein, e.g., Delta3-Ig, can be used to modulate an activity of a toporythmic protein, e.g., Notch. For example, a soluble Delta3 protein can be a stimulatory form of a Delta3 protein, i.e., a Delta3 protein which is capable of stimulating an activity of a toporythmic protein. In one embodiment, such a protein acts in essentially the same manner as wild-type Delta3. In another embodiment, a soluble Delta3 protein is an inhibitory form of a Delta3 protein, i.e., a Delta3 protein which is capable of inhibiting an activity of a toporythmic protein. For example, such a Delta3 protein could inhibit the interaction of wild-type Delta3 with the toporythmic protein. In a preferred embodiment, an inhibitory form of a Delta3 protein inhibits the interaction of several proteins which normally interact with a toporythmic protein, by, e.g., binding to a site of the toporythmic protein that is also a binding site to various other proteins, e.g., other Delta proteins. Accordingly, a Delta3 therapeutic can generally affect the interaction of various toporythmic proteins with each other. Similarly, based at least in part on the sequence and structural similarities between Delta proteins, a Delta therapeutic, other than a Delta3 therapeutic, can also be used for modulating the interaction between a Delta3 protein and a Delta3 interacting binding molecule.

The compounds of the invention can be identified using various assays depending on the type of compound and activity of the compound that is desired. Set forth below are at least some assays that can be used for identifying Delta3 therapeutics. It is within the skill of the art to design additional assays for identifying Delta therapeutics, e.g., Delta3 therapeutics.

By making available purified and recombinant Delta3 polypeptides, the present invention facilitates the development of assays which can be used to screen for drugs, including Delta3 variants, which are either agonists or antagonists of the normal cellular function of the subject Delta3 polypeptides, or of their role in the pathogenesis of cellular differentiation and/or proliferation and disorders related thereto. In one embodiment, the assay evaluates the ability of a compound to modulate binding between a Delta3 polypeptide and a molecule, be it protein or DNA, that interacts either upstream or downstream of the Delta/Notch signaling pathway. A variety of assay formats will suffice and, in light of the present inventions, will be comprehended by a skilled artisan.

4.6.1 Cell-Free Assays

Cell free assays can be used to identify compounds which interact with a Delta3 protein. Such assays are available for testing compounds which are proteins, e.g., toporythmic proteins or variants thereof, as well as for testing compounds which are peptidomimetics, small molecules or nucleic acids. The specific assay used for testing these compounds may vary with the type of compound.

In one embodiment, a compound that interacts with a Delta3 protein is identified by screening, e.g., a library of compounds, for binding to a recombinant or purified Delta3 protein or at least a portion thereof. Such assays can involve labeling one or the two components and measuring the extent of their interaction, by, e.g.,determining the level of the one or two labels. In these assays, it may be preferable to attach the Delta3 protein to a solid phase surface. Methods for achieving this are further described infra. In one embodiment, the library of compounds is a library of small molecules. In another embodiment, the library of compounds is a library of Delta3 variants, which can be produced according to methods described infra.

Identification of a compound which inhibits an interaction between a Delta3 protein and a toporythmic protein can also be performed by screening compounds using aggregation assays, as described, e.g., in Fehon et al. (1990) Cell 61:523-534.

In another embodiment, the invention provides methods for identifying compounds which inhibit the interaction of a Delta3 protein with a molecule, e.g., a toporythmic protein or a protein interacting with the cytoplasmic domain of a Delta3 protein. Such methods, which are preferably used in high throughput assays can be performed as follows.

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as “primary” screens in that they can be generated to permit rapid development and relatively easy detection of an alteration in a molecular target which is mediated by a test compound. Moreover, the effects of cellular toxicity and/or bioavailability of the test compound can be generally ignored in the in vitro system, the assay instead being focused primarily on the effect of the drug on the molecular target as may be manifest in an alteration of binding affinity with upstream or downstream elements. Accordingly, in an exemplary screening assay of the present invention, the compound of interest is contacted with proteins which may function upstream (including both activators and repressors of its activity) or to proteins or nucleic acids which may function downstream of the Delta3 polypeptide, whether they are positively or negatively regulated by it. For example, a protein functioning upstream of a Delta3 polypeptide can be a compound interacting with the extracellular portion of the Delta3 molecule. A protein functioning downstream of a Delta3 polypeptide can be a protein interacting with the cytoplasmic domain of Delta3 and, e.g., transducing a signal to the nucleus. To the mixture of the compound and the upstream or downstream element is then added a composition containing a Delta3 polypeptide. Detection and quantification of complexes of Delta3 with it's upstream or downstream elements provide a means for determining a compound's efficacy at inhibiting (or potentiating) complex formation between Delta3 and the Delta-binding elements. The efficacy of the compound can be assessed by generating dose response curves from data obtained using various concentrations of the test compound. Moreover, a control assay can also be performed to provide a baseline for comparison. In the control assay, isolated and purified Delta3 polypeptide is added to a composition containing the Delta-binding element, and the formation of a complex is quantitated in the absence of the test compound.

Complex formation between the Delta3 polypeptide and a Delta3 binding element may be detected by a variety of techniques. Modulation of the formation of complexes can be quantitated using, for example, detectably labeled proteins such as radiolabeled, fluorescently labeled, or enzymatically labeled Delta3 polypeptides, by immunoassay, or by chromatographic detection.

Typically, it will be desirable to immobilize either Delta3 or its binding protein to facilitate separation of complexes from uncomplexed forms of one or both of the proteins, as well as to accommodate automation of the assay. Binding of Delta3 to an upstream or downstream element, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/Delta3 (GST/Delta) fusion proteins can be adsorbed onto glutathione sepharose beads (Sigma Chemical, St. Louis, Mo.) or glutathione derivatized microtitre plates, which are then combined with the cell lysates, e.g., an ³⁵S-labeled, and the test compound, and the mixture incubated under conditions conducive to complex formation, e.g., at physiological conditions for salt and pH, though slightly more stringent conditions may be desired. Following incubation, the beads are washed to remove any unbound label, and the matrix immobilized and radiolabel determined directly (e.g., beads placed in scintilant), or in the supernatant after the complexes are subsequently dissociated. Alternatively, the complexes can be dissociated from the matrix, separated by SDS-PAGE, and the level of Delta-binding protein found in the bead fraction quantitated from the gel using standard electrophoretic techniques such as described in the appended examples.

Other techniques for immobilizing proteins on matrices are also available for use in the subject assay. For instance, either Delta3 or its cognate binding protein can be immobilized utilizing conjugation of biotin and streptavidin. For instance, biotinylated Delta3 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with Delta3 but which do not interfere with binding of upstream or downstream elements can be derivatized to the wells of the plate, and Delta3 trapped in the wells by antibody conjugation. As above, preparations of a Delta-binding protein and a test compound are incubated in the Delta-presenting wells of the plate, and the amount of complex trapped in the well can be quantitated. Exemplary methods for detecting such complexes, in addition to those described above for the GST-immobilized complexes, include immunodetection of complexes using antibodies reactive with the Delta3 binding element, or which are reactive with Delta3 protein and compete with the binding element; as well as enzyme-linked assays which rely on detecting an enzymatic activity associated with the binding element, either intrinsic or extrinsic activity. In the instance of the latter, the enzyme can be chemically conjugated or provided as a fusion protein with the Delta-BP. To illustrate, the Delta-BP can be chemically cross-linked or genetically fused with horseradish peroxidase, and the amount of polypeptide trapped in the complex can be assessed with a chromogenic substrate of the enzyme, e.g., 3,3′-diaminobenzadine terahydrochloride or 4-chloro-1-naphthol. Likewise, a fusion protein comprising the polypeptide and glutathione-S-transferase can be provided, and complex formation quantitated by detecting the GST activity using 1-chloro-2,4-dinitrobenzene (Habig et al (1974) J Biol Chem 249:7130).

For processes which rely on immunodetection for quantitating one of the proteins trapped in the complex, antibodies against the protein, such as anti-Delta3 antibodies, can be used. Alternatively, the protein to be detected in the complex can be “epitope tagged” in the form of a fusion protein which includes, in addition to the Delta3 sequence, a second polypeptide for which antibodies are readily available (e.g., from commercial sources). For instance, the GST fusion proteins described above can also be used for quantification of binding using antibodies against the GST moiety. Other useful epitope tags include myc-epitopes (e.g., see Ellison et al. (1991) J Biol Chem 266:21150-21157) which includes a 10-residue sequence from c-myc, as well as the pFLAG system (International Biotechnologies, Inc.) or the pEZZ-protein A system (Pharmacia, NJ).

4.6.2. Cell Based Assays

In addition to cell-free assays, such as described above, the readily available source of Delta3 proteins provided by the present invention also facilitates the generation of cell-based assays for identifying small molecule agonists/antagonists and the like. For example, cells which are sensitive to bFGF/VEGF or matrigel can be caused to overexpress a recombinant Delta3 protein in the presence and absence of a test agent of interest, with the assay scoring for modulation in Delta3 responses by the target cell mediated by the test agent. As with the cell-free assays, agents which produce a statistically significant change in Delta-dependent responses (either inhibition or potentiation) can be identified. In an illustrative embodiment, the expression or activity of a Delta3 is modulated in embryos or cells and the effects of compounds of interest on the readout of interest (such as tissue differentiation, proliferation, tumorigenesis) are measured. For example, the expression of genes which are up- or down-regulated in response to a Delta-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected.

Exemplary cell lines may include endothelial cells such as MVEC's and bovine aortic endothelial cells (BAEC's); as well as generic mammalian cell lines such as HeLa cells and COS cells, e.g., COS-7 (ATCC®# CRL-1651). Further, the transgenic animals discussed herein may be used to generate cell lines, containing one or more cell types involved in cardiovascular disease, that can be used as cell culture models for this disorder. While primary cultures derived from the transgenic animals of the invention may be utilized, the generation of continuous cell lines is preferred. For examples of techniques which may be used to derive a continuous cell line from the transgenic animals, see Small et al., 1985, Mol. Cell Biol. 5:642-648.

In one embodiment, a test compound that modifies a Delta3 activity can be identified by incubating a cell having a Delta3 protein with the test compound and measuring signal transduction from the Delta3 protein. Comparison of the signal transduction in the cells incubated with or without the test compound will reveal whether the test compound is a Delta3 therapeutic. Similarly, a test compound that modifies a Delta3 activity can be identified by incubating a cell having a Delta3 ligand with the test compound, e.g., a Delta3 derived compound, and measuring signal transduction from the Delta3 ligand. Comparison of the signal transduction in the cells incubated with or without the test compound will reveal whether the test compound is a Delta3 therapeutic.

In the event that the Delta3 proteins themselves, or in complexes with other proteins, are capable of binding DNA and/or modifying transcription of a gene, a transcriptional based assay could be used, for example, in which a Delta3 responsive regulatory sequence is operably linked to a detectable marker gene, e.g., a luciferase gene. Similarly, Delta3 therapeutics could also be identified by using an assay in which expression of genes that are modulated upon binding of a Delta3 protein to a Delta3 ligand on a cell is monitored. Genes that are responsive to interaction with a Delta3 protein or Delta3 ligand can be identified according to methods known in the art, e.g., differential hybridization or differential display.

In another embodiment, a silicon-based device, called a microphysiometer, can be used to detect and measure the response of cells having a Delta3 protein to test compounds to identify Delta3 therapeutics. This instrument measures the rate at which cells acidify their environment, which is indicative of cellular growth and/or differentiation (McConnel et al. (1992) Science 257:1906).

Monitoring the influence of compounds on cells may be applied not only in basic drug screening, but also in clinical trials. In such clinical trials, the expression of a panel of genes may be used as a “read out” of a particular drug's therapeutic effect.

In yet another aspect of the invention, the subject Delta3 polypeptides can be used to generate a “two hybrid” assay (see, for example, U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), for isolating coding sequences for other cellular proteins which bind to or interact with Delta3 (“Delta-binding proteins” or “Delta-bp”), such as Notch, and the like.

Briefly, the two hybrid assay relies on reconstituting in vivo a functional transcriptional activator protein from two separate fusion proteins. In particular, the method makes use of chimeric genes which express hybrid proteins. To illustrate, a first hybrid gene comprises the coding sequence for a DNA-binding domain of a transcriptional activator fused in frame to the coding sequence for a Delta3 polypeptide. The second hybrid protein encodes a transcriptional activation domain fused in frame to a sample gene from a cDNA library. If the bait and sample hybrid proteins are able to interact, e.g., form a Delta-dependent complex, they bring into close proximity the two domains of the transcriptional activator. This proximity is sufficient to cause transcription of a reporter gene which is operably linked to a transcriptional regulatory site responsive to the transcriptional activator, and expression of the reporter gene can be detected and used to score for the interaction of the Delta3 and sample proteins. This system can be used to identify compounds which modify, e.g., inhibit the interaction between a Delta3 protein and another protein, by adding 1 test compound to a cell containing the above-described plasmids. The effect of the test compound on the reporter gene expression and then measured to determine the effect of the test compound on the interaction.

In another embodiment, the invention provides arrays for identifying compounds that can induce apoptosis of cells through a Delta3 protein. Apoptotic arrays are known in the act and are described, e.g., in Grimm et al. (1996) Proc. Natl. Acad. Sci. USA 93:10923.

4.7 Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

4.7.1 Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sublocalization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes (CITE), and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Furthermore, the nucleic acid sequences disclosed herein can be used to perform searches against “mapping databases”, e.g., BLAST-type search, such that the chromosome position of the gene is identified by sequence homology or identity with known sequence fragments which have been mapped to chromosomes.

A polypeptide and fragments and sequences thereof and antibodies specific thereto can be used to map the location of the gene encoding the polypeptide on a chromosome. This mapping can be carried out by specifically detecting the presence of the polypeptide in embers of a panel of somatic cell hybrids between cells of a first species of animal from which the protein originates and cells from a second species of animal and then determining which somatic cell hybrid(s) expresses the polypeptide and noting the chromosome(s) from the first species of animal that it contains. For examples of this technique, see Pajunen et al. (1988) Cytogenet. Cell Genet. 47:37-41 and Van Keuren et al. (1986) Hum. Genet. 74:34-40. Alternatively, the presence of the polypeptide in the somatic cell hybrids can be determined by assaying an activity or property of the polypeptide, for example, enzymatic activity, as described in Bordelon-Riser et al. (1979) Somatic Cell Genetics 5:597-613 and Owerbach et al. (1978) Proc. Natl. Acad. Sci. USA 75:5640-5644.

4.7.2 Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences of SEQ ID NO: 1 and 24, can comfortably provide positive individual identification with a panel of perhaps 10 to 1,000 primers which each yield a noncoding amplified sequence of 100 bases. If predicted coding sequences, such as those in SEQ ID NO: 3 and 25 are used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

4.7.3 Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e., another DNA sequence that is unique to a particular individual). As mentioned above, actual base sequence information can be used for identification as an accurate alternative to patterns formed by restriction enzyme generated fragments. Sequences targeted to noncoding regions are particularly appropriate for this use as greater numbers of polymorphisms occur in the noncoding regions, making it easier to differentiate individuals using this technique. Examples of polynucleotide reagents include the nucleic acid sequences of the invention or portions thereof, e.g., fragments derived from noncoding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

4.7.4 Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

Another aspect of the invention provides methods for expression of a nucleic acid or polypeptide of the invention or activity of a polypeptide of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of a polypeptide of the invention in clinical trials. These and other agents are described in further detail in the following sections.

4.7.5 Prognostic Assays

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al. (1996) Human Mutation 7:244-255. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication NO: WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions.

In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair enzymes”) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic -acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene encoding a polypeptide of the invention. Furthermore, any cell type or tissue, preferably peripheral blood leukocytes, in which the polypeptide of the invention is expressed may be utilized in the prognostic assays described herein.

4.7.6 Pharmacogenomics

Agents, or modulators which have a stimulatory or inhibitory effect on activity or expression of a polypeptide of the invention as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

4.7.7 Monitoring of Effects During Clinical Trials

Monitoring the influence of agents (e.g., drugs, compounds) on the expression or activity of a polypeptide of the invention (e.g., the ability to modulate aberrant cell proliferation chemotaxis, and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the preadministration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to increase the expression or activity of the polypeptide to higher levels than detected, i.e., to increase the effectiveness of the agent. Alternatively, decreased administration of the agent may be desirable to decrease expression or activity of the polypeptide to lower levels than detected, i.e., to decrease the effectiveness of the agent.

4.8 Transgenic Animals

These systems may be used in a variety of applications. For example, the cell- and animal-based model systems may be used to further characterize Delta3 genes and proteins. In addition, such assays may be utilized as part of screening strategies designed to identify compounds which are capable of ameliorating disease symptoms. Thus, the animal- and cell-based models may be used to identify drugs, pharmaceuticals, therapies and interventions which may be effective in treating disease.

4.8.1. Animal-Based Systems

One aspect of the present invention concerns transgenic animals which are comprised of cells (of that animal) which contain a transgene of the present invention and which preferably (though optionally) express an exogenous Delta3 protein in one or more cells in the animal. A Delta3 transgene can encode the wild-type form of the protein, or can encode homologs thereof, including both alleles of Delta3 genes, agonists and antagonists, as well as antisense constructs. In preferred embodiments, the expression of the transgene is restricted to specific subsets of cells, tissues or developmental stages utilizing, for example, cis-acting sequences that control expression in the desired pattern. In the present invention, such mosaic expression of a Delta3 protein can be essential for many forms of lineage analysis and can additionally provide a means to assess the effects of, for example, lack of Delta3 expression which might grossly alter development in small patches of tissue within an otherwise normal embryo. In a preferred embodiment, the invention provides transgenic mice having an allele of hDelta3 gene which is associated with ACCPN and the mouse can be used, e.g., to determine the effect of this specific hDelta3 allele. Toward this and, tissue-specific regulatory sequences and conditional regulatory sequences can be used to control expression of the transgene in certain spatial patterns. Moreover, temporal patterns of expression can be provided by, for example, conditional recombination systems or prokaryotic transcriptional regulatory sequences.

Genetic techniques which allow for the expression of transgenes can be regulated via site-specific genetic manipulation in vivo are known to those skilled in the art. For instance, genetic systems are available which allow for the regulated expression of a recombinase that catalyzes the genetic recombination a target sequence. As used herein, the phrase “target sequence” refers to a nucleotide sequence that is genetically recombined by a recombinase. The target sequence is flanked by recombinase recognition sequences and is generally either excised or inverted in cells expressing recombinase activity. Recombinase catalyzed recombination events can be designed such that recombination of the target sequence results in either the activation or repression of expression of one of the subject Delta3 proteins. For example, excision of a target sequence which interferes with the expression of a recombinant Delta3 gene, such as one which encodes an antagonistic homolog or an antisense transcript, can be designed to activate expression of that gene. This interference with expression of the protein can result from a variety of mechanisms, such as spatial separation of the Delta3 gene from the promoter element or an internal stop codon. Moreover, the transgene can be made wherein the coding sequence of the gene is flanked by recombinase recognition sequences and is initially transfected into cells in a 3′ to 5′ orientation with respect to the promoter element. In such an instance, inversion of the target sequence will reorient the subject gene by placing the 5′ end of the coding sequence in an orientation with respect to the promoter element which allow for promoter driven transcriptional activation.

The transgenic animals of the present invention all include within a plurality of their cells a transgene of the present invention, which transgene alters the phenotype of the “host cell” with respect to regulation of cell growth, death and/or differentiation. Since it is possible to produce transgenic organisms of the invention utilizing one or more of the transgene constructs described herein, a general description will be given of the production of transgenic organisms by referring generally to exogenous genetic material. This general description can be adapted by those skilled in the art in order to incorporate specific transgene sequences into organisms utilizing the methods and materials described below.

In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation expression of a recombinant Delta3 protein can be regulated via control of recombinase expression.

Use of the cre/loxP recombinase system to regulate expression of a recombinant Delta3 protein requires the construction of a transgenic animal containing transgenes encoding both the Cre recombinase and the subject protein. Animals containing both the Cre recombinase and a recombinant Delta3 gene can be provided through the construction of “double” transgenic animals. A convenient method for providing such animals is to mate two transgenic animals each containing a transgene, e.g., a Delta3 gene and recombinase gene.

One advantage derived from initially constructing transgenic animals containing a Delta3 transgene in a recombinase-mediated expressible format derives from the likelihood that the subject protein, whether agonistic or antagonistic, can be deleterious upon expression in the transgenic animal. In such an instance, a founder population, in which the subject transgene is silent in all tissues, can be propagated and maintained. Individuals of this founder population can be crossed with animals expressing the recombinase in, for example, one or more tissues and/or a desired temporal pattern. Thus, the creation of a founder population in which, for example, an antagonistic Delta3 transgene is silent will allow the study of progeny from that founder in which disruption of Delta3 mediated induction in a particular tissue or at certain developmental stages would result in, for example, a lethal phenotype.

Similar conditional transgenes can be provided using prokaryotic promoter sequences which require prokaryotic proteins to be simultaneous expressed in order to facilitate expression of the Delta3 transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the trans-activating protein, e.g., a recombinase or a prokaryotic protein, is delivered to the tissue and caused to be expressed, such as in a cell-type specific manner. By this method, a Delta3 transgene could remain silent into adulthood until “turned on” by the introduction of the trans-activator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for micro-injection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of a Delta3 protein (either agonistic or antagonistic), and antisense transcript, or a Delta3 mutant. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten et al. (1985) PNAS 82:6148-6152; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) Nature 298:623-628).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

In one embodiment, gene targeting, which is a method of using homologous recombination to modify an animal's genome, can be used to introduce changes into cultured embryonic stem cells. By targeting a Delta3 gene of interest in ES cells, these changes can be introduced into the germlines of animals to generate chimeras. The gene targeting procedure is accomplished by introducing into tissue culture cells a DNA targeting construct that includes a segment homologous to a target Delta3 locus, and which also includes an intended sequence modification to the Delta3 genomic sequence (e.g., insertion, deletion, point mutation). The treated cells are then screened for accurate targeting to identify and isolate those which have been properly targeted.

Gene targeting in embryonic stem cells is in fact a scheme contemplated by the present invention as a means for disrupting a Delta3 gene function through the use of a targeting transgene construct designed to undergo homologous recombination with one or more Delta3 genomic sequences. The targeting construct can be arranged so that, upon recombination with an element of a Delta3 gene, a positive selection marker is inserted into (or replaces) coding sequences of the targeted Delta3 gene. The inserted sequence functionally disrupts the Delta3 gene, while also providing a positive selection trait. Exemplary Delta3 targeting constructs are described in more detail below.

Generally, the embryonic stem cells (ES cells ) used to produce the knockout animals will be of the same species as the knockout animal to be generated. Thus for example, mouse embryonic stem cells will usually be used for generation of knockout mice.

Embryonic stem cells are generated and maintained using methods well known to the skilled artisan such as those described by Doetschman et al. (1985) J. Embryol. Exp. Morphol. 87:27-45). Any line of ES cells can be used, however, the line chosen is typically selected for the ability of the cells to integrate into and become part of the germ line of a developing embryo so as to create germ line transmission of the knockout construct. Thus, any ES cell line that is believed to have this capability is suitable for use herein. One mouse strain that is typically used for production of ES cells, is the 129J strain. Another ES cell line is murine cell line D3 (American Type Culture Collection, catalog NO: CKL 1934) Still another preferred ES cell line is the WW6 cell line (Ioffe et al. (1995) PNAS 92:7357-7361). The cells are cultured and prepared for knockout construct insertion using methods well known to the skilled artisan, such as those set forth by Robertson in: Teratocarcinomas and Embryonic Stem Cells: A Practical Approach, E. J. Robertson, ed. IRL Press, Washington, D.C. [1987]); by Bradley et al. (1986) Current Topics in Devel. Biol. 20:357-371); and by Hogan et al. (Manipulating the Mouse Embryo: A Laboratory Manual, Cold Spring Harbor Laboratory Press, Cold Spring Harbor, N.Y. [1986]).

Insertion of the knockout construct into the ES cells can be accomplished using a variety of methods well known in the art including for example, electroporation, microinjection, and calcium phosphate treatment. A preferred method of insertion is electroporation.

Each knockout construct to be inserted into the cell must first be in the linear form. Therefore, if the knockout construct has been inserted into a vector (described infra), linearization is accomplished by digesting the DNA with a suitable restriction endonuclease selected to cut only within the vector sequence and not within the knockout construct sequence.

For insertion, the knockout construct is added to the ES cells under appropriate conditions for the insertion method chosen, as is known to the skilled artisan. Where more than one construct is to be introduced into the ES cell, each knockout construct can be introduced simultaneously or one at a time.

If the ES cells are to be electroporated, the ES cells and knockout construct DNA are exposed to an electric pulse using an electroporation machine and following the manufacturer's guidelines for use. After electroporation, the ES cells are typically allowed to recover under suitable incubation conditions. The cells are then screened for the presence of the knockout construct.

Screening can be accomplished using a variety of methods. Where the marker gene is an antibiotic resistance gene, for example, the ES cells may be cultured in the presence of an otherwise lethal concentration of antibiotic. Those ES cells that survive have presumably integrated the knockout construct. If the marker gene is other than an antibiotic resistance gene, a Southern blot of the ES cell genomic DNA can be probed with a sequence of DNA designed to hybridize only to the marker sequence Alternatively, PCR can be used. Finally, if the marker gene is a gene that encodes an enzyme whose activity can be detected (e.g., b-galactosidase), the enzyme substrate can be added to the cells under suitable conditions, and the enzymatic activity can be analyzed. One skilled in the art will be familiar with other useful markers and the means for detecting their presence in a given cell. All such markers are contemplated as being included within the scope of the teaching of this invention.

The knockout construct may integrate into several locations in the ES cell genome, and may integrate into a different location in each ES cell's genome due to the occurrence of random insertion events. The desired location of insertion is in a complementary position to the DNA sequence to be knocked out, e.g., the Delta3 coding sequence, transcriptional regulatory sequence, etc. Typically, less than about 1-5% of the ES cells that take up the knockout construct will actually integrate the knockout construct in the desired location. To identify those ES cells with proper integration of the knockout construct, total DNA can be extracted from the ES cells using standard methods. The DNA can then be probed on a Southern blot with a probe or probes designed to hybridize in a specific pattern to genomic DNA digested with particular restriction enzyme(s). Alternatively, or additionally, the genomic DNA can be amplified by PCR with probes specifically designed to amplify DNA fragments of a particular size and sequence (i.e., only those cells containing the knockout construct in the proper position will generate DNA fragments of the proper size).

After suitable ES cells containing the knockout construct in the proper location have been identified, the cells can be inserted into an embryo. Insertion may be accomplished in a variety of ways known to the skilled artisan, however a preferred method is by microinjection. For microinjection, about 10-30 cells are collected into a micropipet and injected into embryos that are at the proper stage of development to permit integration of the foreign ES cell containing the knockout construct into the developing embryo. For instance, as the appended Examples describe, the transformed ES cells can be microinjected into blastocytes.

The suitable stage of development for the embryo used for insertion of ES cells is very species dependent, however for mice it is about 3.5 days. The embryos are obtained by perfusing the uterus of pregnant females. Suitable methods for accomplishing this are known to the skilled artisan, and are set forth by, e.g., et al. (1986) Current Topics in Devel. Biol. 20:357-371.

While any embryo of the right stage of development is suitable for use, preferred embryos are male. In mice, the preferred embryos also have genes coding for a coat color that is different from the coat color encoded by the ES cell genes. In this way, the offspring can be screened easily for the presence of the knockout construct by looking for mosaic coat color (indicating that the ES cell was incorporated into the developing embryo). Thus, for example, if the ES cell line carries the genes for white fur, the embryo selected will carry genes for black or brown fur.

After the ES cell has been introduced into the embryo, the embryo may be implanted into the uterus of a pseudopregnant foster mother for gestation. While any foster mother may be used, the foster mother is typically selected for her ability to breed and reproduce well, and for her ability to care for the young. Such foster mothers are typically prepared by mating with vasectomized males of the same species. The stage of the pseudopregnant foster mother is important for successful implantation, and it is species dependent. For mice, this stage is about 2-3 days pseudopregnant.

Offspring that are born to the foster mother may be screened initially for mosaic coat color where the coat color selection strategy (as described above, and in the appended examples) has been employed. In addition, or as an alternative, DNA from tail tissue of the offspring may be screened for the presence of the knockout construct using Southern blots and/or PCR as described above. Offspring that appear to be mosaics may then be crossed to each other, if they are believed to carry the knockout construct in their germ line, in order to generate homozygous knockout animals. Homozygotes may be identified by Southern blotting of equivalent amounts of genomic DNA from mice that are the product of this cross, as well as mice that are known heterozygotes and wild type mice.

Other means of identifying and characterizing the knockout offspring are available. For example, Northern blots can be used to probe the mRNA for the presence or absence of transcripts encoding either the gene knocked out, the marker gene, or both. In addition, Western blots can be used to assess the level of expression of the Delta3 gene knocked out in various tissues of the offspring by probing the Western blot with an antibody against the particular Delta3 protein, or an antibody against the marker gene product, where this gene is expressed. Finally, in situ analysis (such as fixing the cells and labeling with antibody) and/or FACS (fluorescence activated cell sorting) analysis of various cells from the offspring can be conducted using suitable antibodies to look for the presence or absence of the knockout construct gene product.

Yet other methods of making knock-out or disruption transgenic animals are also generally known. See, for example, Manipulating the Mouse Embryo, (Cold Spring Harbor Laboratory Press, Cold Spring Harbor, N.Y., 1986). Recombinase dependent knockouts can also be generated, e.g., by homologous recombination to insert target sequences, such that tissue specific and/or temporal control of inactivation of a Delta-gene can be controlled by recombinase sequences (described infra).

Animals containing more than one knockout construct and/or more than one transgene expression construct are prepared in any of several ways. The preferred manner of preparation is to generate a series of mammals, each containing one of the desired transgenic phenotypes. Such animals are bred together through a series of crosses, backcrosses and selections, to ultimately generate a single animal containing all desired knockout constructs and/or expression constructs, where the animal is otherwise congenic (genetically identical) to the wild type except for the presence of the knockout construct(s) and/or transgene(s).

The present invention is further illustrated by the following examples which should not be construed as limiting in any way. The contents of all cited references (including literature references, issued patents, published patent applications as cited throughout this application are hereby expressly incorporated by reference. The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### 5. EXAMPLES

5.1 Isolation of a Full-Length cDNA Encoding Human Delta3

Human microvascular endothelial cells (HMVEC catalog #CC2543; Clonetics, San Diego, Calif.) were separated into four samples of cells which were treated as follows. The first sample was untreated. The second sample was treated with human TGF-β1 (hTGF-β1) (10 ng/ml) (Upstate Biotechnology, Lake Placid, N.Y., Catalog NO: 01-134). The third sample was treated with bFGF (10 ng/ml)VEGF (25ng/ml) (Upstate Biotechnology, Lake Placid, N.Y., Catalog NO: 01-134, Catalog Nos. 01-106 and 01-185, respectively). The fourth sample was differentiated on Matrigel (Collaborative Biomedical Products, Becton Dickinson Labware, Bedford, Mass.). Cells were treated as indicated for 24 hours, the 4 samples were pooled, and RNA was extracted from the pooled cells using a QIAGEN RNeasy kit. The resulting cDNA library was subjected to high throughput random sequencing. This allowed identification of a cDNA fragment comprising the following 171 nucleotide long sequence:

(SEQ ID NO: 21)

GCCCAGGCNGACCCTGGTGTGGACTGTGAGCTGGAGCTCAGCGAGTGTGA
CAGCAACCCCTGTCGCANTGGAGGCAGCTGTAAGGACCANGAGGATGGCT
ACCACTGCCTGTGTCCTCCGGGCTACTACGGCNTGCATCGTGAACACNGC
ACCTCTTAGCTGNGCCGACTC.

Comparison of the nucleotide sequence of this partial cDNA with the sequences in GenBank using the BLAST program (Altschul et al. (1990) J. Mol. Biol. 215:403) revealed that the nucleotide sequence encoded a protein fragment having a significant homology to Delta proteins. In fact, the amino acid sequence had significant homology with a chicken Delta1 protein (GenBank Accession NO: U26590), a Xenopus Delta1 protein (GenBank Accession NO: L42229), a rat Delta1 protein (GenBank Accession NO: U78889), a Xenopus Delta2 protein (GenBank Accession NO: U70843) as well as Notch proteins.

A full-length cDNA of about 3.2 kb was then isolated by screening a human microvascular endothelial cell (HMVEC) cDNA library using the partial cDNA (SEQ ID NO: 21). This nucleic acid was deposited at the American Type Culture Collection (ATCC®) on Mar. 5, 1997, and has been assigned ATCC® Accession NO: 98348. The nucleotide sequence of the cDNA isolated is shown in FIG. 1 and has SEQ ID NO: 1.

A nucleic acid sequence comparison of SEQ ID NO: 1 against EST sequence databases using the BLAST program (Altschul et al. (1990) J. Mol. Biol. 215:403) indicated that 5 ESTs have a homology to portions of SEQ ID NO: 1. These are all located 3′ of the nucleotide sequence encoding the transmembrane domain, i.e., downstream of nucleotide 1996 of SEQ ID NO: 1. Three of these ESTs (having accession Nos. T33770, T33811, and T07963) have a nucleotide sequence starting at about nucleotide 2044 of SEQ ID NO: 1. However, the nucleotide sequence of the three EST is significantly different from the nucleotide sequence of hDelta3 in about the first 50 nucleotides 3′ of nucleotide 2044 of SEQ ID NO: 1. Two ESTs (having Accession Nos. R32717 and T07962) are located further downstream of the three ESTs.

The nucleic acid having SEQ ID NO: 1 encodes a protein of 685 amino acids having SEQ ID NO: 2. A comparison of the amino acid sequence of SEQ ID NO: 2 with sequences in GenBank using BLASTP (Altschul et al. (1990) J. Mol. Biol. 215:403) reveals that this protein has a certain homology to previously described Delta proteins. FIG. 2 shows an alignment of the human Delta3 protein having SEQ ID NO: 2 with the amino acid sequence of mouse Delta1 protein (Accession NO: X80903), rat Delta1 protein (Accession NO: U78889), chicken Delta1 protein (Accession NO: U26590), two Xenopus Delta1 proteins (Accession Nos. L42229 and U70843) and Drosophila Delta1 protein (Accession NO: AA142228). The sequence comparison indicates that human Delta3 protein has the general structure of a Delta3 protein. In particular, human Delta3 protein has a signal peptide corresponding to about amino acid 1 to about amino acid 17 of SEQ ID NO: 2, a DSL motif corresponding to the sequence from about amino acid 173 to about amino acid 217, a first EGF-like domain corresponding to the sequence from about amino acid 222 to about amino acid 250, a second EGF-like domain corresponding to the sequence from about amino acid 253 to about amino acid 281, a third EGF-like domain corresponding to the sequence from about amino acid 288 to about amino acid 321, a fourth EGF-like domain corresponding to the sequence from about amino acid 328 to about amino acid 359, a fifth EGF-like domain corresponding to the sequence from about amino acid 366 to about amino acid 399, a sixth EGF-like domain corresponding to the sequence from about amino acid 411 to about amino acid 437, a seventh EGF-like domain corresponding to the sequence from about amino acid 444 to about amino acid 475, an eight EGF-like domain corresponding to the sequence from about amino acid 484 to about amino acid 517, a transmembrane domain corresponding to the sequence from about amino acid 530 to about amino acid 553, and a cytoplasmic domain corresponding to the sequence from about amino acid 554 to about amino acid 685 of SEQ ID NO: 2.

An amino acid and nucleotide sequence comparison between the members of the Delta1 and Delta3 protein family and human Delta3 on one hand and between the members of the Delta1 family reveals that the homology between the Delta3 family members is stronger than the homology between human Delta3 and any of the Delta1 family members. For example, although hDelta3 is only approximately 58% similar to the Drosophila Delta1 protein; approximately 70% similar to the mouse Delta1 protein; approximately 70% similar to the rat Delta1 protein; approximately 68% similar to the chick Delta1 protein; and approximately 68% similar to the Xenopus Delta1 proteins; the drosophila, mouse, rat, chick and Xenopus Delta1 proteins are very similar to each other (e.g., the mouse and rat Delta1 are about 96% similar to each other). Published PCT application WO97/01571 discloses a partial nucleotide and amino acid sequence of a protein having significant homology to Delta1 family members, indicating that it is likely to be a human Delta1 protein. The homology between the partial amino acid sequence of human Delta1 and the amino acid sequence of human Delta3 is indicated in Table I and shows that the proteins are encoded by different genes. All these amino acid and nucleotide sequence comparisons indicate that human Delta3 is an additional species of Delta proteins, sharing some sequence and structure homology with the Delta1 proteins.

In one embodiment of a nucleotide sequence of human Delta3, the nucleotide at position 455 is a guanine (G)(SEQ ID NO: 1). In this embodiment, the amino acid at position 40 is glutamate (E)(SEQ ID NO: 2). In another embodiment of a nucleotide sequence of human Delta3, the nucleotide at position 455 is a cytosine (C)(SEQ ID NO: 27). In this embodiment, the amino acid at position 40 is glutamine (Q)(SEQ ID NO: 28). In another embodiment of a nucleotide sequence of human Delta3, the nucleotide at position 455 is a thymidine (T)(SEQ ID NO: 29). In this embodiment, the amino acid at position 40 is a stop codon (SEQ ID NO: 30). In another embodiment of a nucleotide sequence of human Delta3, the nucleotide at position 455 is a adenine (A)(SEQ ID NO: 31). In this embodiment, the amino acid at position 40 is lysine (K)(SEQ ID NO: 32).

In one embodiment of a nucleotide sequence of human Delta3, the nucleotide at position 786 is an cytosine (C)(SEQ ID NO: 1). In this embodiment, the amino acid at position 150 is a alanine (A)(SEQ ID NO: 2). In an alternative embodiment, a species variant of human Delta3 has a nucleotide at position 786 which is a thymidine (T)(SEQ ID NO: 33). In this embodiment, the amino acid at position 150 is valine (V)(SEQ ID NO: 34), i.e., a conservative substitution.

In one embodiment of a nucleotide sequence of human Delta3, the nucleotide at position 594 is a cytosine (C)(SEQ ID NO: 1). In this embodiment, the amino acid at position 86 is threonine (T)(SEQ ID NO: 2). In an alternative embodiment, a species variant of human Delta3 has a nucleotide at position 594 which is a guanine (G)(SEQ ID NO: 35). In this embodiment, the amino acid at position 86 is serine (S)(SEQ ID NO: 36), i.e., a conservative substitution.

In one embodiment of a nucleotide sequence of human Delta3, wherein the nucleotide at position 883 is a thymidine (T)(SEQ ID NO: 1). In this embodiment, the amino acid at position 182 is aspartate (D)(SEQ ID NO: 2). In an alternative embodiment, a species variant of human Delta3 has a nucleotide at position 883 which is an adenine (A)(SEQ ID NO: 37). In this embodiment, the amino acid at position 182 is glutamate (E)(SEQ ID NO: 38), i.e., a conservative substitution.

5.2. Isolation of a Full-Length cDNA Encoding Mouse Delta3

A mouse Delta3 cDNA was identified from mouse lung database library of expressed sequences using the human Delta3 cDNA (SEQ ID NO: 1) as a query sequence. The most homologous sequence, SEQ ID NO: 24 was identified as a 3.2 kb cDNA.

The nucleic acid having SEQ ID NO: 24 encodes a protein of 686 amino acids having the amino acid sequence shown in SEQ ID NO: 25. FIG. 4 shows the nucleic acid sequence of mouse Delta3 and FIG. 4 shows the amino acid sequence. FIG. 5 shows an alignment of the human and mouse Delta3 proteins having SEQ ID NO: 2 and 25, respectively. The sequence comparison indicates that human and mouse Delta3 proteins have significant similarity and identity (i.e., 88.2% similar and 86.6% identical) suggesting evolutionary conservation due to an essential biological function.

Mouse Delta3 protein has a signal peptide corresponding to about amino acid 1 to about amino acid 17 of SEQ ID NO: 25, a DSL motif corresponding to the sequence from about amino acid 174 to about amino acid 218, a first EGF-like domain corresponding to the sequence from about amino acid 223 to about amino acid 251, a second EGF-like domain corresponding to the sequence from about amino acid 254 to about amino acid 282, a third EGF-like domain corresponding to the sequence from about amino acid 289 to about amino acid 322, a fourth EGF-like domain corresponding to the sequence from about amino acid 329 to about amino acid 360, a fifth EGF-like domain corresponding to the sequence from about amino acid 367 to about amino acid 400, a sixth EGF-like domain corresponding to the sequence from about amino acid 412 to about amino acid 438, a seventh EGF-like domain corresponding to the sequence from about amino acid 445 to about amino acid 476, an eight EGF-like domain corresponding to the sequence from about amino acid 485 to about amino acid 518, a transmembrane domain corresponding to the sequence from about amino acid 531 to about amino acid 554, and a cytoplasmic domain corresponding to the sequence from about amino acid 555 to about amino acid 686 of SEQ ID NO: 25.

In one embodiment of a nucleotide sequence of mouse Delta3, the nucleotide at position 49 is cytosine (C)(SEQ ID NO: 24). In this embodiment, the amino acid at position 4 is alanine (A)(SEQ ID NO: 25). In an alternative embodiment, a species variant of mouse Delta3 has a nucleotide at position 49 which is thymidine (T)(SEQ ID NO: 39). In this embodiment, the amino acid at position 4 is valine (V)(SEQ ID NO: 40), i.e., a conservative substitution.

In one embodiment of a nucleotide sequence of mouse Delta3, the nucleotide at position 51 is thymidine (T)(SEQ ID NO: 24). In this embodiment, the amino acid at position 5 is serine (S)(SEQ ID NO: 25). In an alternative embodiment, a species variant of mouse Delta3 has a nucleotide at position 51 which is a adenine (A)(SEQ ID NO: 41). In this embodiment, the amino acid at position 5 is threonine (T)(SEQ ID NO: 42), i.e., a conservative substitution.

In one embodiment of a nucleotide sequence of mouse Delta3, the nucleotide at position 109 is guanine (G)(SEQ ID NO: 24). In this embodiment, the amino acid at position 24 is arginine (R)(SEQ ID NO: 25). In an alternative embodiment, a species variant of mouse Delta3 has a nucleotide at position 109 which is adenine (A)(SEQ ID NO: 43). In this embodiment, the amino acid at position 24 is histidine (H)(SEQ ID NO: 44), i.e., a conservative substitution.

In one embodiment of a nucleotide sequence of mouse Delta3, wherein the nucleotide at position 130 is a thymidine (T)(SEQ ID NO: 24). In this embodiment, the amino acid at position 31 is phenylalanine (F)(SEQ ID NO: 25). In an alternative embodiment, a species variant of mouse Delta3 has a nucleotide at position 130 which is adenine (A)(SEQ ID NO: 45). In this embodiment, the amino acid at position 31 is tyrosine (Y)(SEQ ID NO: 46), i.e., a conservative substitution.

5.3 Tissue Expression of the hDelta3 Gene

This Example describes the tissue distribution of Delta3 protein, as determined by Northern blot hybridization with a 1.6 kb fragment of human Delta3 cDNA corresponding to the extreme 3′ end of SEQ ID NO: 1 and by in situ hybridization using a probe complementary to nucleotides 1290-1998 of SEQ ID NO: 1.

Northern blot hybridizations with the various RNA samples were performed under standard conditions and washed under stringent conditions, i.e., in 0.2× SSC at 65^{□}C. In each sample, the probe hybridized to a single RNA of about 3.5 kb. The results of hybridization of the probe to various mRNA samples are described below.

Hybridization of a Clontech Fetal Multiple Tissue Northern (MTN) blot (Clontech, LaJolla, Calif.) containing RNA from fetal brain, lung, liver, and kidney indicated the presence of Delta3 RNA in each of these fetal tissues. Expression was significantly higher in fetal lung and kidney than in fetal brain and liver. Hybridization of a Clontech human Multiple Tissue Northern I (MTNI) and Multiple Tissue Northern II (MTNII) blots (Clontech, LaJolla, Calif.) containing RNA from adult heart, brain, placenta, lung, liver, skeletal muscle, kidney, pancreas, spleen , thymus, prostate, testis, ovary, small intestine, mucosal lining of the colon, and peripheral blood leukocytes with the human 1.6 kb Delta3 probe indicated expression in heart, placenta, lung, skeletal muscle, kidney, pancreas, spleen, thymus, prostate, testis, ovary, small intestine and colon. Expression was particularly strong in adult heart, placenta, lung, and skeletal muscle. Expression was also found in adult brain, liver and testis. However, no significant amount of hDelta3 mRNA was detected in adult peripheral blood leukocytes.

Further, Northern blot hybridization of total mRNA from HMVEC cells treated with TGF-β1 at 10 ng/ml for 24 hours, bFGF at 10 ng/ml/VEGF at 25 ng/ml for 24 hours, or untreated for 24 hours indicated that Delta3 expression was induced upon induction with bFGF/VEGF. Accordingly, expression of Delta3 is up-regulated in HMV endothelial cells in response to certain growth factors.

Hybridization of a “cancer” Northern blot containing RNA from HL-60, HeLa, K562, MoLT4, Raji, SW480, A549, and G361 cells, revealed that Delta3 is expressed at high levels in the colorectal carcinoma cell line SW480. Thus, Delta3 expression is high in at least certain tumor cells.

Delta-3 in situs on paraffin embedded mouse embryos were performed. Expression was seen in endothelial cells of the secondary vasculature and in preendothelial cells in the bone marrow. There is no expression in endothelial cells after day P 1.5.

For in situ hybridization analysis of mDelta3, 10 m sagittal sections of fresh frozen day E13.5, E14.5, E15.5, E16.5, E18.5 and P1.5 embryos of B6 mice, as well as 8 m cross sections of brain, spinal cord, eye and harderian gland , submandibular gland, white fat, stomach, heart, lung, liver, spleen, thymus, small intestine, lymph node, pancreas, skeletal muscle, testes, ovary, placenta, kidney and adrenal gland from adult B6 mice. were used for hybridization. Sections were postfixed with 4% formaldehyde in DEPC-treated 1× phosphate-buffered saline at room temperature for 10 minutes before being rinsed twice in DEPC-treated 1× phosphate-buffered saline and once in 0.1 M triethanolamine-HCl (pH8.0). Following incubation in 0.25% acetic anhydride-0.1 M triethanolamine-HCl for 10 minutes, sections were rinsed in DEPC-treated 2× SSC (1× SSC is 0.15M NaCl plus 0.015M sodium citrate). Tissue was dehydrated through a series of ethanol washes, incubated in 100% chloroform for 5 minutes, and then rinsed in 100% ethanol for 1 minute and 95% ethanol for 1 minute and allowed to air dry.

The hybridization was performed using a ³⁵S-radiolabeled cRNA probe from the DNA sequence of nucleotides 1290-1998 of SEQ ID NO:1.

Tissues were incubated with probe (approximately 5×10⁷ cpm/ml) in the presence of a solution containing 600 mM NaCl, 10 mM Tris (pH 7.5), 1 mM EDTA, 0.01% sheared salmon sperm DNA, 0.01% yeast tRNA, 0.05% yeast total RNA type X1, 1× Denhardt's solution, 50% formamide, 10% dextran sulfate, 100 mM dithiothreitol, 0.1% sodium dodecylsulfate (SDS), and 0.1% sodium thiosulfate for 18 h at 55 C.

After hybridization, slides were washed with 2× SSC. Sections were then sequentially incubated at 37 C. in TNE (a solution containing 10 mM Tris-HCl (pH 7.6), 500 mM NaCl, and 1 mM EDTA), for 10 minutes, in TNE with 10 ug of RNase A per ml for 30 minutes, and finally in TNE for 10 minutes. Slides were then rinsed with 2× SSC at room temp, washed with 2× SSC at 50° C. for 1 hour, washed with 0.2× SSC at 55° C. for 1 hour, and 0.2× SSC at 60° C. for 1 hour. Sections were then dehydrated rapidly through serial ethanol-0.3 M sodium acetate concentrations before being air dried and exposed to Kodak Biomax MR scientific imaging film for 6 days at room temperature.

Expression was most abundant and wide spread during embryogenesis. Strongest expression was observed in the eye in all of the embryonic ages tested. Signal in a pattern suggestive of neuronal expression was not observed in any other tissues making the expression in the eye unique. Moderate ubiquitous expression was also detected in lung, thymus and brown fat during embryogenesis. A multifocal, scattered signal was also observed throughout the embryo. This signal pattern was more focused in the cortical region of the kidney and outlining the intestinal tract. Adult expression was highest in the ovary and the cortical regions of the kidney and adrenal gland.

Thus, Delta3 is expressed in numerous tissues, but is not detected in certain tissues, e.g., peripheral blood leukocytes and adult heart tissue (at least when using Northern blot hybridization), is expressed at relatively high levels in at least some tumor cells, e.g., colon carcinoma cells, and its expression can be up-regulated in response to some growth factors, e.g., bFGF and VEGF. Furthermore, in situ hybridization shows that mDelta3 is expressed most strongly in developing tissues of eye, thymus, lung and brown fat.

A Southern blot containing DNA from a panel of a human/hamster mono-chromosomal somatic cell hybrids was probed with an hDelta1 cDNA probe. The results obtained clearly indicates that the human Delta3 gene resides on chromosome 15.

5.4 Increased Expression of hDelta3 in Differentiating Endothelial Cells

This Example shows that the expression of the hDelta3 gene increases in differentiating endothelial cells relative to non-differentiating endothelial cells.

HMVEC cells were separated into 5 cultures and treated as follows: (1) cells were induced to quiescence by growth in basal endothelial growth medium (EGM) (Clontech) which contains 10% fetal calf serum (FCS); (2) cells were grown in complete endothelial growth medium (EGM-MV) (Clontech, Catalog NO: CC-3125) which contains 10% FCS and growth factors; (3) cells were stimulated to proliferate by culture in EGM-MV in the presence of bFGF at 10 ng/ml and VEGF at 25 ng/ml; (4) cells were stimulated to proliferate by culture in EGM-MV in the presence of TGF-β1 at 10 ng/ml; and (5) cells were stimulated to differentiate by culture in EGM-MV on Matrigel. After 24 hours of culture, the cells were harvested, the RNA was extracted and submitted to Northern blot analysis. Hybridization was performed with the 1.6 kb hDelta3 probe described above. The results indicate that among the culture conditions tested, quiescent cells express the lowest amount of hDelta3 (at a barely detectable level). Cells which are proliferating express a higher level of hDelta3. Interestingly, the mRNA level of hDelta3 was strongly increased in cells induced to differentiate by plating on Matrigel.

Thus, this Example clearly demonstrates that hDelta3 expression is strongly increased in cells induced to differentiate and also in cells induced to proliferate.

5.5 hDelta3 is Located in a Chromosomal Region Associated with ACCPN

The location of hDelta3 on human chromosome 15 was determined using radiation hybrid (RH) mapping.

A sequence tagged site (STS) was generated from the 3′ untranslated region of the gene using a forward primer having the nucleotide sequence GTTTACATTGCATCCTGGAT (SEQ ID NO: 51) and a reverse primer having the nucleotide sequence CTCTTCTGTTCCTCTGGTTG (SEQ ID NO: 22). The STS was used to screen the Genebridge 4 (Gyapay et al. (1996) Human Molecular Genetics 5:339) and the Standford G3 (Stewart et al. (1997) Genome Res. 7:422) radiation hybrid panels. These panels were derived by fusion of irradiated human donor cells with rodent recipient cells and can be used for positioning STS markers within existing framework maps, ordering markers in the region of interest as well as establishing the distance between markers.

RH mapping was performed by PCR under the following conditions: 25 ng DNA/20 μl reaction, 0.5 μM of each primer, 0.2 mM of each nucleotide, 1.5 mM MgCl₂, 1× buffer as provided by the manufacturer of the enzyme, 35 cycles at 94□C., 55□C., 72□C. for 30 seconds each.

The results of the RH mapping indicated that hDelta3 maps to 15q12-15 close to framework marker D15S1244 on the Stanford G3 panel and close to framework marker D15S144 on the Genebridge 4 panel with a LOD score >3. Searching of the OMIM database (Online Mendelian Inheritance in Man; http://www.ncbi.nlm.nih.gob/Omim/searchomim.html) indicated that this region has previously been genetically linked to a neurological disorder called Agenesis of the Corpus Callosum with Peripheral Neuropathy (ACCPN) (Casaubon et al. (1996) Am. J. Hum. Genet. 58:28).

5.6 Delta3 Encodes a Notch Ligand

The example presented herein demonstrates that Delta3 encodes a Notch Ligand. In particular, the data presented herein shows, first, that hDelta3 encodes a functional Notch ligand as determined by its ability to block differentiation of C2C12 cells. When C2C12 cells are co-cultured, under low mitogenic conditions, with NIH3T3 cells expressing a Notch ligand, the differentiation to myotubes by the C2C12 cells is blocked. (Lindsell et al. (1995) Cell 80:909). If the cells differentiate troponin T is expressed, if differentiation is blocked no troponin T expression is seen. In addition, the data presented herein directly demonstrates that Delta3 binds Notch one and Notch 2 Third, the data presented in this section identifies several cell types that endogenously exhibit Delta3 receptors.

To determine whether hDelta3 in fact encodes a functional Notch ligand, NIH3T3 cells were engineered to express hDelta3, co-cultured with C2C12 cells and analyzed for troponin T expression. Briefly, NIH3T3 cells were infected with a retrovirus containing the hDelta3 coding region cloned into the MIGR retroviral vector (Pear et al. (1998) Blood 92:3780). This vector contains an Internal Ribosome Entry Site (IRES) downstream of the cloning site, followed by the cDNA for the green fluorescent protein (GFP). GFP expression from the vector is monitored to assess the efficiency of transduction of the vector into the target cells. C2C12 cells were plated in 10 cm dishes and cultured in DMEM media with 10%

Inactivated Fetal Calf Serum (10% IFS) until 70% confluent. C2C12 cells were then washed 1× with PBS. 5×10⁶ NIH3T3 cells harboring either an empty vector, a vector expressing hDelta3 or a vector expressing Jagged-1 were resuspended in 10 mls DMEM media containing 10% Horse serum (10% HS), and laid on top of the C2C12 cells.

Control experiments involved the solitary culture of C2C12 cells in differentiation media (10% HS) as well as in growth media (10% IFS). The whole population of cells was lysed three to four days later and equal amounts of protein was resolved on an SDS-polyacrylamide gel. The proteins were then transferred onto a nitrocellulose membrane and probed with an anti-Troponin T antibody (Sigma, 1:200). A secondary incubation with an anti-mouse antibody conjugated to horseradish peroxidase allowed for detection using chemiluminescence reagents (Amersham). When cells NIH3T3 cells containing the empty vector were co-cultured with C2C12 cells, troponin T was expressed, indicating that the C2C12 cells had indeed differentiated into myotubes. When NIH3T3 cells expressing hDelta3 were co-cultured with C2C12 cells, no expression of troponin T was seen, indicating that C2C12 differentiation was blocked by hDelta3. This result is similar to that seen when NIH3T3 cells expressing Jagged 1 (a functional Notch ligand).

Next, Delta3 was tested for its ability to bind human notch1 and notch2. 293T cells were transiently transfected with expression plasmids (PCMV poly-neo) encoding full-length Notch1 or Notch2. Two days after transfection cells were incubated with purified protein consisting of the extracellular domain of hDelta3 fused in frame to the Fc portion of immunoglobulin G (hDelta3-Fc) at 10 μg/ml or with control protein consisting of human immunoglobulin G1 (hIgG1) at 10 ug/ml in stainig buffer (PBS containing 3% fetal calf serum, 1 mM CaCl₂ and 0.02% sodium azide). After one hour of incubation, cells were washed three times in staining buffer and bound protein was detected by incubating the cells with FITC-conjugated anti-human IgG for 30 minutes. Cells were analyzed under fluorescence microscopy.

Binding of hDelta3-Fc fusions to cells expressing Notch1 and Notch2 but not to cells transfected with empty expression vector, was detected. Binding was calcium-dependent and was abolished in the presence of 5 mM EDTA. Control-Fc fusions and hIgG1 did not show any binding to transfected cells. These results establish hDelta3 as a ligand for Notch1 and Notch2 and show that the extracellular domain of hDelta3 is suffcient to mediate binding to Notch.

Therefore, Delta3, including hDelta3, represents a polypeptide which can function as a bona fide Notch ligand.

Next, Cell lines were tested for the presence of an endogenous receptor for hDelta3. Briefly, cells were washed two times in staining buffer and incubated with hDelta3-Fc or hIgG1 (10 μg/ml in staining buffer) at a cell concentration of 5×10⁶ per ml. After an incubation of one hour on ice, cells were washed three times in staining buffer and bound protein was detected by incubating the cells with FITC-conjugated secondary antibody (anti-human hIgG1) for 30 min on ice. Cells were analyzed by flow cytometry on a FACSCalibur.

Binding of hDelta3-Fc was seen to Jurkat, 32D, C2C12 and Cos cells. A control Fc fusion protein and hIgG1 did not show binding to these cell lines. The binding of hDelta3-Fc was dependent on calcium since the binding was abolished by the addition of 5 mM EDTA to the binding buffer.

5.7. Delta3 Affects Early Development and Muscle Cell Differentiation

The data presented herein demonstrate that among the roles of Delta3 is a function that involves early development and muscle cell differentiation.

Materials and Methods

Preparation of hDelta3 RNA: The template for the hDelta3 in vitro transcription reaction was prepared from the DNA construct containing the hDelta3 sequence inserted in a pCS2++ vector, which was then linearized using AscI. Capped RNA was synthesized using SP6 RNA polymerase from the linearized plasmid using mMESSAGE mMACHINE kit (Ambion, Austin, Tex.) according to the manufacturer's instructions. In vitro transcribed capped RNA was purified using RNAesy kit (Qiagen) and analyzed by gel electrophoresis.

HDelta3 RNA injection into Xenopus embryos: Xenopus embryos were obtained by in vitro fertilization, dejellied in 2% cysteine HCl (pH 7.6), washed thoroughly in Modified Ringer's solution, and incubated at 15-25° C. Embryos were transferred to injection solution (Modified Ringer's solution containing 3% Ficoll) prior to injections. One ng and 2.5 ng of hDelta3 RNA were injected into one blastomere at the 2-cell stage. Embryos were transferred to 0.1× MMR from the injection solution after approximately 6 hours and grown until the appropriate stage.

Embryos for histological examination were fixed in 4% formaldehyde in PBS overnight, embedded in paraffin and stained with Heidenhain's Azan stain by standard procedures. Transverse sections of injected embryos show disruption of somitic organization and somite boundaries on the injected side.

Xenopus animal cap assay: 2 ng of hDelta3 RNA was injected into the animal pole of each of the 2 Xenopus blastomeres at the 2-cell stage. Animal caps from uninjected or injected embryos were explanted at stage 9 and cultured in 1× Modified Ringers containing 0.01% BSA and 50 ug/ml gentamycin. Animal caps were cultured until control embryos have reached stage23-24. Animal cap tissue was lysed and total RNA was extracted using RNeasy kit (Qiagen). RT-PCRs were performed on these samples using gene-specific primers and appropriate annealing temperatures and the products analyzed by gel electrophoresis. The primers used in this experiment were specific to genes EF1-alpha, XCG-1, NCAM, Xbra, M-actin, Sox-17 (Amaravadi et al. (1997). Dev. Biol. 192:392-404). RT-PCR analysis did not indicate expression of any of the specific marker genes tested.

Results:

Examination of embryos injected with hDelta3 RNA two days post-injection showed an overexpression phenotype involving axial disruption indicative of an effect on somites and anterior dorsal structures such as eyes and cement glands were not well differentiated in half of the injected embryos. These results suggest that hDelta3 has an effect on early tissue development/differentiation.

The differential stain used in this study also indicated an enlargement of somite size on the injected side demonstrating that hDelta3 has an effect on muscle cells and overexpression can lead to enlarged muscle mass. Notch/Delta signaling has been shown to play a key role in somitogenesis/myogenesis in various species (Wittenberger et al., (1999) EMBO J. 18:915-922); Dornseifer et al. (1997) Mech. Dev. 63:159-171); Kusumi et al. (1998) Nat. Genet. 19:274-278).

Therefore, the results presented herein indicate that Delta3 can be involved in early development (e.g., can have a role downstream of germ layer specification function), and can be involved in modulating myogenesis and muscle cell differentiation.

5.8 Identification of Delta Therapeutics

This Example describes a simple assay for isolating Delta therapeutics, (e.g., agonist or antagonist of a Delta activity), e.g., Delta3 therapeutics. Based at least in part on the results described in the previous Examples, Delta therapeutics can be used for treating various diseases, including neurological diseases, and/or hyper- or hypoproliferative diseases, hematologic disorders , immunodeficiency states and diseases or conditions associated with defects in vasculature and/or conditions requiring neovascularization and/or conditions hallmarked by aberrant neovascularization, e.g., diabetic retinopathy. In addition, based at least in part on the similarity of amino acid sequence and structure between the various Delta proteins, Delta3 therapeutics can be used to treat diseases or conditions associated with an aberrant Delta3 activity or an aberrant Delta activity other than a Delta3 activity. Similarly, Delta3 therapeutics as well as Delta therapeutics other than Delta3 therapeutics can be used to treat diseases or conditions associated with an aberrant Delta3 activity. The assay set forth below is applicable to Delta proteins other than Delta3 proteins.

A Delta3 therapeutic can be identified by using an in vitro assay, in which the interaction between a Delta3 protein and a Delta3 binding protein, e.g., a Notch protein, is determined in the presence and in the absence of a test compound. A soluble binding fragment of a Delta3 protein can be prepared by expression of the extracellular portion of human Delta3, e.g., about amino acids 1-529 of SEQ ID NO: 2 or about amino acids 1-530 of SEQ ID NO: 25, in E. coli according to methods known in the art. Alternatively, the Delta3 protein fragment can be about amino acid 173 to about amino acid 517 of SEQ ID NO: 2 or from about amino acid 174 to about amino acid 508 of SEQ ID NO: 25. Similarly, a Delta3 binding fragment of a Delta3 binding protein (i.e., Delta3 binding partner) can be produced recombinantly.

A Delta3 binding protein can be a Notch protein and can be identified, e.g., by determining whether the protein is capable of binding to a Delta3 protein. A nucleic acid encoding a Notch protein can be obtained, e.g., by PCR amplifying a portion of a Notch gene encoding at least an EGF-like domain, using primers having a nucleotide sequence derived from the nucleotide sequence of a Notch gene present in GenBank or disclosed in PCT Application NO: PCT/US92/03651 or PCT/US93/09338.

Test compounds can then be tested to determine whether they inhibit the interaction between the Delta3 and the Delta3 binding protein by using an ELISA type assay. Accordingly, one of the recombinantly produced Delta3 protein and the Delta3 binding protein, e.g., Notch protein, is attached to a solid phase surface and the other protein is labeled, e.g., such as by tagging the protein with an epitope, for which an antibody is available (e.g., FLAG epitope, available from International Biotechnologies, Inc.). As a non-limiting example of an assay, the Delta3 protein can be linked to the wells of a microtiter (96 well) plate by overnight incubation of the protein at a concentration of 10 μg/ml in PBS. After blocking unoccupied sites on the plate with a BSA solution, various amounts of test compounds and the recombinantly produced Delta3 binding protein are added to the wells in a buffer suitable for a specific interaction between the proteins.

After an incubation time of several hours, the wells are rinsed with buffer, and the amount of Delta3 binding protein attached to the wells is determined. The amount of bound protein can be determined by incubating the wells with an anti-tag, e.g., anti-myc, antibody, which can then be detected by enzyme immunoassay. The amount of bound protein is then determined by determining the optical density using an ELISA reader. A lower amount of Delta3 binding protein in a well that contained a test compound relative to a well that did not contain a test compound is indicative that the test compound inhibits the interaction between Delta3 and a Delta3 binding protein.

In a further non-limiting example of a binding assay, a recombinantly produced and labeled Delta3 polypeptide, or fragment thereof capable of binding a Delta3 binding protein, is incubated, with or without a test compound, with cells expressing the Delta3 binding protein (Shimizu et al. (1999) J. Biol. Chem. 274:32961-32969). Alternatively, the recombinant Delta3 polypeptide is not labeled and is detected upon binding the cell by a second Delta3 binding protein, such as an antibody. A lower amount of Delta3 binding protein in a well that contained a test compound relative to a well that did not contain a test compound is indicative that the test compound inhibits the interaction between Delta3 and a Delta3 binding protein.

A Delta3 therapeutic can also be identified by using a reporter assay in which the level of expression of a reporter construct under the control of a Delta3 promoter is measured in the presence or absence of a test compound. A Delta3 promoter can be isolated by screening a genomic library with a Delta3 cDNA which preferably contains the 5′ end of the cDNA. A portion of the Delta3 promoter, typically from about 50 to about 500 base pairs long is then cloned upstream of a reporter gene, e.g., a luciferase gene, in a plasmid. This reporter construct is then transfected into cells, e.g., neural cells or endothelial cells. Transfected cells are then be distributed into wells of a multiwell plate and various concentrations of test compounds are added to the wells. After several hours incubation, the level of expression of the reporter construct is determined according to methods known in the art. A difference in the level of expression of the reporter construct in transfected cells incubated with the test compound relative to transfected cells incubated without the test compound will indicate that the test compound is capable of modulating the expression of the Delta3 gene and is thus a Delta3 therapeutic.

Deposit of Microorganisms

A nucleic acid encoding a full-length human Delta protein is contained in a plasmid which was deposited with the American Type Culture Collection, 10801 University Boulevard, Manassas, Va. 20110-2209 (ATCC®) on Mar. 5, 1997 and has been assigned ATCC® accession number 98348.

Equivalents

Those skilled in the art will recognize, or be able to ascertain using no more than routine experimentation, many equivalents of the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed is:

1. An isolated nucleic acid molecule selected from the group consisting of:
a) a nucleic acid molecule comprising a nucleotide sequence which is at least 80% identical to the nucleotide sequence of SEQ ID NO: 1 or SEQ ID NO:3;
b) a nucleic acid molecule comprising a fragment of at least 500 nucleotides of the nucleotide sequence of SEQ ID NO:1 or SEQ ID NO:3;
c) a nucleic acid molecule which encodes a polypeptide comprising the amino acid sequence of SEQ ID NO:2;
d) a nucleic acid molecule which encodes a fragment of a polypeptide comprising the amino acid sequence of SEQ ID NO:2, wherein the fragment comprises at least 100 contiguous amino acids of SEQ ID NO:2; and
e) a nucleic acid molecule which encodes a naturally occurring allelic variant of a polypeptide comprising the amino acid sequence of SEQ ID NO:2, wherein the nucleic acid molecule hybridizes to a nucleic acid molecule comprising SEQ ID NO:1, 3, or a complement thereof, under stringent conditions.

2. The isolated nucleic acid molecule of claim 1, further comprising a fragment of at least 1000 nucleotides of the nucleotide sequence of SEQ ID NO:1 or SEQ ID NO:3.

3. The isolated nucleic acid molecule of claim 1, further comprising a fragment of at least 1500 nucleotides of the nucleotide sequence of SEQ ID NO:1 or SEQ ID NO:3.

4. The isolated nucleic acid molecule of claim 1, further comprising a fragment of at least 2000 nucleotides of the nucleotide sequence of SEQ ID NO:1 or SEQ ID NO:3.

5. The isolated nucleic acid molecule of claim 1, which encodes a fragment of a polypeptide comprising the amino acid sequence of SEQ ID NO:2, wherein the fragment comprises at least 100 contiguous amino acids of SEQ ID NO:2.

6. The isolated nucleic acid molecule of claim 1, which encodes a fragment of a polypeptide comprising the amino acid sequence of SEQ ID NO:2, wherein the fragment comprises at least 200 contiguous amino acids of SEQ ID NO:2.

7. The isolated nucleic acid molecule of claim 1, which is selected from the group consisting of:
a) a nucleic acid comprising the nucleotide sequence of SEQ ID NO:1 or SEQ ID NO:3; and
b) a nucleic acid molecule which encodes a polypeptide comprising the amino acid sequence of SEQ ID NO:2.

8. The nucleic acid molecule of claim 1 further comprising vector nucleic acid sequences.

9. The nucleic acid molecule of claim 1 further comprising nucleic acid sequences encoding a heterologous polypeptide.

10. A host cell which contains the nucleic acid molecule of claim 1.

11. The host cell of claim 10 which is a mammalian host cell.

12. A non-human mammalian host cell containing the nucleic acid molecule of claim 1.

13. An isolated polypeptide selected from the group consisting of:
a) a polypeptide which is encoded by a nucleic acid molecule comprising a nucleotide sequence which is at least 80% identical to a nucleic acid comprising the nucleotide sequence of SEQ ID NO:1, SEQ ID NO:3, or a complement thereof;
b) a naturally occurring allelic variant of a polypeptide comprising the amino acid sequence of SEQ ID NO:2, wherein the polypeptide is encoded by a nucleic acid molecule which hybridizes to a nucleic acid molecule comprising SEQ ID NO:1 or SEQ ID NO:3; and
c) a fragment of a polypeptide comprising the amino acid sequence of SEQ ID NO:2, wherein the fragment comprises at least 100 contiguous amino acids of SEQ ID NO:2.

14. The isolated polypeptide of claim 13, comprising a fragment which comprises at least 100 contiguous amino acids of SEQ ID NO:2.

15. The isolated polypeptide of claim 13, comprising a fragment which comprises at least 200 contiguous amino acids of SEQ ID NO:2.

16. The isolated polypeptide of claim 13, comprising a fragment which comprises at least 500 contiguous amino acids of SEQ ID NO:2.

17. The isolated polypeptide of claim 13, comprising a fragment which is at least 90% homologous to the amino acid sequence of SEQ ID NO:2.

18. The isolated polypeptide of claim 13, comprising a fragment which is at least 95% homologous to the amino acid sequence of SEQ ID NO:2.

19. The isolated polypeptide of claim 13, comprising the amino acid sequence of SEQ ID NO:2.

20. The polypeptide of claim 13 further comprising heterologous amino acid sequences.

21. An antibody which selectively binds to a polypeptide of claim 13.

22. The antibody of claim 21, which is a monoclonal antibody.

23. The antibody of claim 22, comprising an immunologically active portion selected from the group consisting of:
a) an scFV fragment;
b) a dcFV fragment;
c) an Fab fragment; and
d) an F(ab′)₂ fragment.

24. The antibody of claim 22, wherein the antibody is selected from the group consisting of:
a) a chimeric antibody;
b) a humanized antibody;
c) a human antibody;
d) a non-human antibody; and
e) a single chain antibody.

25. A method for producing a polypeptide selected from the group consisting of:
a) a polypeptide comprising the amino acid sequence of SEQ ID NO:2;
b) a polypeptide comprising a fragment of the amino acid sequence of SEQ ID NO:2, wherein the fragment comprises at least 100 contiguous amino acids of SEQ ID NO:2;
c) a naturally occurring allelic variant of a polypeptide comprising the amino acid sequence of SEQ ID NO:2, or the amino acid sequence encoded by the cDNA insert of the plasmid deposited with the ATCC as Accession Number ______, wherein the polypeptide is encoded by a nucleic acid molecule which hybridizes to a nucleic acid molecule comprising SEQ ID NO:1, SEQ ID NO:3, or a complement thereof under stringent conditions;
comprising culturing the host cell of claim 10 under conditions in which the nucleic acid molecule is expressed.

26. A method for detecting the presence of a polypeptide of claim 13 in a sample, comprising:
contacting the sample with a compound which selectively binds to a polypeptide of claim 13; and
determining whether the compound binds to the polypeptide in the sample.

27. The method of claim 26, wherein the compound which binds to the polypeptide is an antibody.

28. A kit comprising a compound which selectively binds to a polypeptide of claim 13 and instructions for use.

29. A method for detecting the presence of a nucleic acid molecule of claim 1 in a sample, comprising the steps of:
contacting the sample with a nucleic acid probe or primer which selectively hybridizes to the nucleic acid molecule; and
determining whether the nucleic acid probe or primer binds to a nucleic acid molecule in the sample.

30. The method of claim 29, wherein the sample comprises mRNA molecules and is contacted with a nucleic acid probe.

31. A kit comprising a compound which selectively hybridizes to a nucleic acid molecule of claim 1 and instructions for use.

32. A method for identifying a compound which binds to a polypeptide of claim 13 comprising the steps of:
contacting a polypeptide, or a cell expressing a polypeptide of claim 13 with a test compound; and
determining whether the polypeptide binds to the test compound.

33. The method of claim 32, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for Delta3-mediated signal transduction.

34. A method for modulating the activity of a polypeptide of claim 13 comprising contacting a polypeptide or a cell expressing a polypeptide of claim 13 with a compound which binds to the polypeptide in a sufficient concentration to modulate the activity of the polypeptide.

35. A method for identifying a compound which modulates the activity of a polypeptide of claim 13, comprising:
contacting a polypeptide of claim 13 with a test compound; and
determining the effect of the test compound on the activity of the polypeptide to thereby identify a compound which modulates the activity of the polypeptide.
